(19) United States
(12) Patent Application Publication
(10) Pub. No.: US 2021/0322785 A1
(43) Pub. Date: Oct. 21, 2021

(54) COMPOSITIONS AND METHODS FOR TREATING WOUNDS

(71) Applicant: C.P. MEDICAL CORPORATION, NORCROSS, GA (US)

(72) Inventor: Linda Black, San Jose, CA (US)

(21) Appl. No.: 17/312,596

(22) PCT Filed: Dec. 10, 2019
(86) PCT No.: PCT/US2019/065362

§ 371 Date: Jun. 10, 2021

Related U.S. Application Data
(60) Provisional application No. 62/777,354, filed on Dec. 10, 2018.

(51) Int. Cl. A61N 5/06; A61L 27/52; A61L 27/18; A61L 27/54; A61N 5/067

(52) U.S. Cl. CPC A61N 5/0616; A61L 27/52; A61L 27/18; A61N 2005/066; A61N 5/067; A61N 2005/0663; A61L 27/54

(57) ABSTRACT

Provided herein are biocompatible hydrogel polymer matrices that can be used with laser, for example, to treat wounds without the removal of the hydrogel.

### CROSS-REFERENCE TO RELATED APPLICATIONS

This application claims priority to U.S. Provisional Application No. 62/777,354, filed Dec. 10, 2018, which is incorporated by reference in its entirety.

This application is also related to PCT Application No. PCT/US2011/035643, filed May 6, 2011, U.S. application Ser. No. 13/696,032, filed Jan. 15, 2013, U.S. application Ser. No. 13/696,028, filed Jan. 16, 2013, U.S. Pat. No. 10,111,985, issued Oct. 30, 2018, U.S. Pat. No. 9,072,809, issued Jul. 7, 2015, U.S. Pat. No. 8,987,339, issued Mar. 24, 2015, U.S. application Ser. No. 15/479,519, filed Apr. 5, 2017, each of which is incorporated herein by reference in its entirety.

### BACKGROUND

Wound treatment still requires extensive therapies to prevent infections and to promote wound healing. Lasers can be used to treat wounds, but it can require the removal of a bandage so that the wound can be exposed to the laser. Therefore, there is a need for a bandage that does not need to be removed in conjunction with a laser treatment. The present embodiments satisfies these needs as well as others.

### SUMMARY

In some embodiments, methods of treating a wound on a subject, the method comprising contacting a wound that is covered by a hydrogel bandage with a laser pulse to treat the wound, wherein the hydrogel bandage is not removed while the laser pulse is applied to the wound. In some embodiments, the hydrogel bandage is a fully synthetic, polyglycol-based biocompatible hydrogel polymer matrix comprising a fully synthetic, polyglycol-based biocompatible hydrogel polymer comprising at least one first monomeric unit bound through at least one amide, thioester, or thioether linkage to at least one second monomeric unit, wherein the polymer forms the matrix covers the wound. In some embodiments, the polyglycol-based biocompatible hydrogel polymer matrix of claim 1, wherein the at least one first monomeric unit is PEG-based and fully synthetic, and wherein the at least one second monomeric unit is PEG-based and fully synthetic. In some embodiments, the first monomeric unit is derived from a MULTIARM-(5-50k)-SH, a MULTIARM-(5-50k)-NH2 or a MULTIARM-(5-50k)-AA monomer and the second monomeric unit is derived from a MULTIARM-(5-50k)-SG, a MULTIARM-(5-50k)-SGA, or a MULTIARM-(5-50k)-SS monomer. In some embodiments, the first monomeric unit is derived from a 4ARM-5k-SH, 4ARM-2k-NH2, 4ARM-5k-NH2, 8ARM-20k-NH2, 4ARM-20k-AA, or 8ARM-20k-AA monomer, and the second monomeric unit is derived from a 4ARM-10k-SG, 8ARM-15k-SG, 4ARM-20k-SGA, or 4ARM-20k-SS monomer. In some embodiments, the hydrogel is formed from 8-ARM-AA-20K, 8-ARM-NH2-20K, and 4-ARM-SGA-20K. In some embodiments, the hydrogel comprises a viscosity enhancing agent, such as HPMC. In some embodiments, the hydrogel comprises a buffer.

### BRIEF DESCRIPTION OF THE DRAWINGS

Error! Reference source not found. shows the effect of addition of degradable acetate amine 8ARM-20k-AA or 4ARM-20k-AA on degradation times. Degradations occurred in phosphate buffered saline (PBS) at 37° C.

FIG. 2 shows the effect of polymer concentration on degradation time for 75% Acetate Amine formulation and 100% Acetate Amine formulation.

FIG. 3 shows the effect of a polymer left in the air as the percent of water weight loss over time.

FIG. 4 shows a sample plot generated by the Texture Analyzer Exponent software running the firmness test. The peak force was recorded as the polymer firmness, which represents the point where the target penetration depth of 4 mm has been reached by the probe.

FIG. 5 shows a sample plot generated by the Texture Analyzer Exponent software running the elastic modulus test under compression. The modulus was calculated from the initial slope of the curve up to 10% of the maximum compression stress.

FIG. 6 shows an exemplary plot generated by the Texture Analyzer Exponent software running the adhesion test. A contact force of 100.0 g was applied for 10 seconds. The tack was measured as the peak force after lifting the probe from the sample. The adhesion energy or the work of adhesion was calculated as the area under the curve representing the tack force (points 1 to 2). The stringiness was defined as the distance traveled by the probe while influencing the tack force (points 1 and 2).

FIG. 7 shows the firmness vs. degradation time plotted as percentages for the polymer formulation: 8ARM-20k-AA/8ARM-20k-NH2 (70/30) & 4ARM-20k-SGA at 4.8% solution with 0.3% HPMC. The error bars represent the standard deviations of 3 samples. The degradation time for the polymer was 18 days.

FIG. 8 shows the chlorhexidine cumulative % elution.

FIG. 9 shows that for a polymer, the triamcinolone cumulative % elution for 60, 90 and 240 day polymers.

FIG. 10 shows that for short degradation time version of the hydrogel polymer loaded with Depo-Medrol, the methylprednisolone cumulative % elution.

FIG. 11 shows that for long degradation time version of a polymer loaded with Depo-Medrol, the methylprednisolone cumulative % elution.

FIG. 12A shows the effect of solid phosphate powder concentration on polymer gel time (A) and solution pH (B).

FIG. 12B shows the effect of solid phosphate powder concentration on solution pH (B).

FIG. 13A shows the effect of sterilization on gel times for polymers of various concentrations.

FIG. 13B shows the effect of sterilization on gel times for polymers of various concentrations.

FIG. 14 shows the storage stability of kits at 5° C., 20° C. and 37° C.

### DETAILED DESCRIPTION OF THE INVENTION

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by 10% and remain within the scope of the disclosed embodiments.

Wound therapy is necessary and lasers have been found to be useful in treating wounds. However, a laser can often not penetrate through a bandage to promote healing. The embodiments provided for herein combine a hydrogel and a laser to treat wounds without removing the hydrogel bandage.

A biocompatible pre-formulation to form a biocompatible hydrogel polymer matrix enables the administration of the laser directly at the wound site and through the hydrogel bandage. This provides the ability to keep the area cleaner and does not require multiple changes of bandages.

In some embodiments, the hydrogel can be formed from a biocompatible pre-formulation that at least in part polymerizes and/or gels to form the biocompatible hydrogel polymer matrix or bandage. In some embodiments, the biocompatible hydrogel matrix comprises a biocompatible hydrogel scaffold. Without being bound to any particular theory, the biocompatible hydrogel polymer matrix provides structural and nutritional support for the wound after administration of the polymer matrix or pre-formulation to a target site, such as the wound, and the laser treatment. In some instances, the hydrogel polymer matrix is biodegradable.

The biocompatible hydrogel polymer matrix comprising may start out as a liquid biocompatible pre-formulation which is delivered to a target site using minimally invasive techniques. Once in or on the body, the liquid formulation polymerizes into a biocompatible hydrogel polymer matrix bandage. In some instances, the biocompatible hydrogel polymer matrix adheres to the tissue. In some instances, the biocompatible hydrogel polymer matrix is delivered to a target site after polymerization. In some instances, polymerization times are controlled by varying the composition of the biocompatible pre-formulation components allowing for the appropriate application and placement of the biocompatible hydrogel polymer matrix. The controlled gelling allows the use of the biocompatible hydrogel polymer matrix to deliver at least one cell directly to the affected target tissue, thereby minimizing systemic exposure. In some embodiments, the biocompatible hydrogel polymer matrix may polymerize outside the body. In certain embodiments, exposure to the cells is limited to the tissue around the target site. In some embodiments, the patient is not exposed systemically to a cell therapy. In certain embodiments, the biocompatible pre-formulation allows the cells to remain viable during and after polymerization. In some embodiments, the cells are combined with a biocompatible hydrogel polymer matrix after polymerization and/or gel formation. In some embodiments, the biocompatible hydrogel polymer matrix further polymerizes and/or gels after delivery to a target site.

Cells may also be administered via a biocompatible hydrogel polymer matrix directly on a wound or surgical site. Biocompatible pre-formulations may form a biocompatible hydrogel polymer matrix that is easily applied on the wound or surgical site and the surrounding skin. The biocompatible hydrogel polymer matrix enables the administration of cells directly to the wound or surgical site. Biocompatible pre-formulations may polymerize and/or gel prior to or after application to the wound or surgical site. In some instances, once the biocompatible pre-formulation is applied, e.g., sprayed over the wound or surgical site, in the liquid form, the biocompatible pre-formulation gels quickly and forms a solid biocompatible hydrogel polymer matrix layer over the wound or surgical site. The biocompatible hydrogel polymer matrix seals the wound or surgical site and it also sticks to the surrounding skin. The biocompatible hydrogel polymer matrix layer over the wound or surgical site acts as a barrier to keep the wound or surgical site from getting infected. In some instances, the biocompatible hydrogel polymer matrix layer in contact with the skin makes the skin surface sticky and thus allows a bandage to stick to the skin more effectively. In some embodiments, the biocompatible hydrogel polymer matrix is non-toxic. After healing has taken place, the biocompatible hydrogel polymer matrix can dissolve and can be absorbed without producing toxic by-products. In some embodiments, the wound or surgical site is healed by the formation of a graft after the administration of stem cells with a biocompatible hydrogel polymer matrix. In certain embodiments, the biocompatible pre-formulation is applied to a wound or surgical site without the cells losing viability. In certain embodiments, the biocompatible hydrogel polymer matrix keeps the wound or surgical site sealed for 24-48 hours and protects it from infection, which avoids repeat visits to the hospital and thus saving costs. In certain embodiments, exposure to the cells is limited to the tissue around the target site. In some embodiments, the patient is not exposed systemically to a cell therapy.

In some embodiments, the biocompatible hydrogel polymer matrix is also loaded with one or more additional components, such as a buffer or a therapeutic agent. The physical and chemical nature of the biocompatible hydrogel polymer matrix is such that a large variety of cell types and additional components may be used with the biocompatible pre-formulation that forms the biocompatible hydrogel polymer matrix. In some embodiments, the additional components enhance the viability and functionality of the cells. In some embodiments, the additional components comprise activation factors. In some embodiments the activation factors include growth factors for cell growth stimulation and proliferation.

In some embodiments, the subject is treated with a laser after the hydrogel matrix is placed at the wound site. In some embodiments, the laser can be as described in Laser Therapy in Canine Rehabilitation, Chapter 21, Darryl L. Millis and Debbie Gross Saunders, October 2013, which is hereby incorporated by reference in its entirety.

In some embodiments, the laser is used in the method at a wavelength of about 630 to about 685 nm or about 700 to about 1000 nm. In some embodiments, the laser is at a wavelength of about 660 nm or about 780 nm. In some embodiments, the laser is at a wavelength of about 650, 810, 980, 915, and the like. In some embodiments, the laser is pulsed for about 1 to about 999 milliseconds. In some embodiments, the laser is used to deliver a total dose of about 20 J/cm². In some embodiments, the laser is used to deliver a dose of about 1.3 J/cm² to about 3 J/cm². In some embodiments, the laser is administered at a dose of about 1 J/cm². In some embodiments, the dosage is anywhere from about 1 J/cm² to about 5 J/cm², including any amount between the endpoints. In some embodiments, the total dose is a therapeutically effective amount for the intended purpose. As used herein, the phrase “therapeutically effective amount” as it relates to the laser refers to the individual dose or the total dose that is delivered through the hydrogel bandage that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment. In some embodiments, the therapeutically effective amount is an amount to prevent or treat an infection or to treat the wound.

In some embodiments, the laser is administered to the subject through the hydrogel bandage. In some embodiments, the laser is administered through the hydrogel bandage once a day for 5 days. In some embodiments, the laser is administered through the hydrogel bandage once a day for 1, 2, 3, 4, 5, 6, 7, 8, 9, 10, 11, 12, 13, or 14 days. In some embodiments, the laser is administered through the bandage once a day for 1, 2, 3, 4, 5, 6, 7, 8, 9, 10, 11, 12, 13, or 14 days without removal of the hydrogel bandage. In some embodiments, the laser is administered through the hydrogel bandage once a day for 1-14, 2-14, 3-14, 4-14, 5-14, 6-14, 7-14, 8-14, 9-14, 10-14, 11-14, or 12-14 days. In some embodiments, the laser is administered through the hydrogel bandage once a day for 1-14, 2-14, 3-14, 4-14, 5-14, 6-14, 7-14, 8-14, 9-14, 10-14, 11-14, or 12-14 days without removal of the hydrogel bandage. In some embodiments, a first hydrogel bandage is applied for a period of time (such as those provided herein) and the wound is treated with a laser through the bandage for a period of time, such as those listed above and herein and then the first bandage is removed and a second hydrogel bandage is applied and the process is repeated. In some embodiments, a hydrogel bandage is applied to the wound and the laser treatment is performed through the bandage for the entire period of time without removal or changing of the hydrogel bandage.

In some embodiments, the use of the laser through the hydrogel bandage results in the stimulation of fibroblast development in the subject at the site of the wound.

In some embodiments, the use of the laser through the hydrogel bandage results in the stimulation of angiogenesis development in the subject at the site of the wound.

In some embodiments, the use of the laser through the hydrogel bandage results in the formation of capillaries in the subject at the site of the wound.

The combination of the laser and they hydrogel bandage can be used to treat various wounds, such as, but not limited to, a burn, traumatic injury, a cut, a laceration, an abrasion, puncture, or an avulsion.

In some embodiments, the hydrogel is not removed and the laser is transmitted or pulsed through the laser. In some embodiments, the hydrogel is partially removed prior to treating a wound with the laser. In some embodiments, laser treats the wound through the hydrogel (e.g. hydrogel bandage). The hydrogel can be any hydrogel provided for herein.

Hydrogels that can be used in the methods provided for herein can be as follows. These are non-limiting examples. Provided herein are biocompatible pre-formulations, comprising at least one first compound comprising more than one nucleophilic group, at least one second compound comprising more than one electrophilic group, optionally at least one cell, and optionally additional components. An exemplary additional component is a culture medium. In certain embodiments, the culture medium is a buffer. In certain embodiments the culture medium contains nutrients for the optional at least one cell. In certain embodiments the optional at least one cell is a stem cell. In certain embodiments, the at least one first compound is formulated in a buffer. In certain embodiments, the at least one second compound is formulated in a buffer. In certain embodiments, the optional at least one cell is formulated in a buffer. In certain embodiments, at least one biocompatible pre-formulation component is a solid. In certain embodiments, all components of the biocompatible pre-formulations are solids. In certain embodiments, at least one biocompatible pre-formulation component is a liquid. In certain embodiments, all biocompatible pre-formulation components are liquids. In certain embodiments, the biocompatible pre-formulation components form a biocompatible hydrogel polymer matrix at a target site by mixing the at least one first compound, the at least one second compound, the optional at least one cell, and the optional additional component in the presence of water and delivering the mixture to the target site such that the biocompatible hydrogel polymer matrix at least in part polymerizes and/or gels at the target site. In certain embodiments, the biocompatible pre-formulation forms a biocompatible hydrogel polymer matrix at a target site by mixing the at least one first compound, the at least one second compound, and the optional at least one cell in the presence of water and delivering the mixture to the target site such that the biocompatible hydrogel polymer matrix at least in part polymerizes and/or gels at the target site. In certain embodiments, the optional additional component, e.g. buffer, is added after the formulation is combined. In certain embodiments, the biocompatible pre-formulation forms a biocompatible hydrogel polymer matrix prior to application at a target site by mixing the at least one first compound, the at least one second compound, the optional at least one cell, and the optional additional component in the presence of water and delivering the mixture to the target site such that the biocompatible hydrogel polymer matrix at least in part polymerizes and/or gels prior to application at a target site. In certain embodiments, the biocompatible pre-formulation forms a biocompatible hydrogel polymer matrix prior to application at a target site by mixing the at least one first compound, the at least one second compound, and the optional at least one cell in the presence of water and delivering the mixture to the target site such that the biocompatible hydrogel polymer matrix at least in part polymerizes and/or gels prior to application at a target site. In certain embodiments, the optional additional component, e.g. buffer, is added after the formulation is combined. In certain embodiments, the biocompatible pre-formulations are biodegradable. In certain embodiments, the biocompatible hydrogel polymer matrix comprises a biocompatible hydrogel scaffold. In certain embodiments, the biocompatible hydrogel scaffold comprises the at least one first compound and the at least one second compound. In certain embodiments, the biocompatible hydrogel scaffold comprises the at least one first compound, the at least one second compound and a buffer. In certain embodiments, the biocompatible hydrogel scaffold is fully synthetic.

Provided herein are biocompatible pre-formulations, comprising at least one first compound comprising more than one nucleophilic group, at least one second compound comprising more than one electrophilic group, a buffer, and optionally additional components. An exemplary additional component is at least one cell. In certain embodiments the cell is a stem cell. In certain embodiments, the buffer is a culture medium. In certain embodiments the culture medium provides nutrients to a cell. In certain embodiments, the at least one first compound is formulated in a buffer. In certain embodiments, the at least one second compound is formulated in a buffer. In certain embodiments, at least one biocompatible pre-formulation component is a solid. In certain embodiments, all biocompatible pre-formulations are solids. In certain embodiments, at least one biocompatible pre-formulation component is a liquid. In certain embodiments, all biocompatible pre-formulation components are liquids. In certain embodiments, the biocompatible pre-formulation forms a biocompatible hydrogel polymer matrix at a target site by mixing the at least one first compound, the at least one second compound, the buffer, and the optional additional component in the presence of water and delivering the mixture to the target site such that the biocompatible hydrogel polymer matrix at least in part polymerizes and/or gels at the target site. In certain embodiments, the biocompatible pre-formulation forms a biocompatible hydrogel polymer matrix at a target site by mixing the at least one first compound, the at least one second compound, and the buffer in the presence of water and delivering the mixture to the target site such that the biocompatible hydrogel polymer matrix at least in part polymerizes and/or gels at the target site. In certain embodiments, the optional additional component, e.g. cell, is added after the formulation is combined. In certain embodiments, the biocompatible pre-formulation forms a biocompatible hydrogel polymer matrix prior to application at a target site by mixing the at least one first compound, the at least one second compound, the buffer, and the optional additional component in the presence of water and delivering the mixture to the target site such that the biocompatible hydrogel polymer matrix at least in part polymerizes and/or gels prior to application at a target site. In certain embodiments, the biocompatible pre-formulation forms a biocompatible hydrogel polymer matrix prior to application at a target site by mixing the at least one first compound, the at least one second compound, and the buffer in the presence of water and delivering the mixture to the target site such that the biocompatible hydrogel polymer matrix at least in part polymerizes and/or gels prior to application at a target site. In certain embodiments, the optional additional component, e.g. cell, is added after the formulation is combined. In certain embodiments, the biocompatible pre-formulations are biodegradable. In certain embodiments, the biocompatible hydrogel polymer matrix comprises a biocompatible hydrogel scaffold. In certain embodiments, the biocompatible hydrogel scaffold comprises the at least one first compound, the at least one second compound and a buffer. In certain embodiments, the biocompatible hydrogel scaffold is fully synthetic.

In some embodiments, the biocompatible pre-formulation compounds comprise monomers which polymerize into polymers. In some embodiments, the biocompatible pre-formulation monomers polymerize to form a biocompatible hydrogel polymer matrix. In some embodiments, a polymer is a biocompatible hydrogel polymer matrix. In some embodiments, a polymer is a biocompatible hydrogel scaffold. In some embodiments, the biocompatible pre-formulation compounds gel to form a biocompatible hydrogel polymer matrix. In some embodiments, the biocompatible pre-formulation compounds gel to form a biocompatible hydrogel scaffold. In some embodiments, the biocompatible pre-formulation compounds polymerize and gel to form a biocompatible hydrogel polymer matrix. In some embodiments, the biocompatible pre-formulation compounds polymerize and gel to form a biocompatible hydrogel polymer scaffold. In some embodiments, the biocompatible hydrogel polymer matrix further polymerizes after hydrogel polymer matrix formation. In some embodiments, the biocompatible hydrogel polymer matrix gels after hydrogel polymer matrix formation. In some embodiments, the biocompatible hydrogel polymer matrix further polymerizes and gels after hydrogel polymer matrix formation.

In some embodiments, the first or second compound comprising more than one nucleophilic or electrophilic group are glycol-based. In some embodiments, glycol-based compounds include ethylene glycol, propylene glycol, butylene glycol, alkyl glycols of various chain lengths, and any combination or copolymers thereof. In some embodiments, the glycol-based compounds are polyglycol-based compounds. In some embodiments, the polyglycol-based compounds include, but are not limited to, polyethylene glycols (PEGs), polypropylene glycols (PPGs), polybutylene glycols (PBGs), and polyglycol copolymers. In some embodiments, glycol-based compounds include polyethylene glycol, polypropylene glycol, polybutylene glycol, polyalkyl glycols of various chain lengths, and any combination or copolymers thereof. In some embodiments, the glycol-based compounds are fully synthetic. In some embodiments, the polyglycol-based compounds are fully synthetic.

In some embodiments, the first or second compound comprising more than one nucleophilic or electrophilic group are polyol derivatives. In certain embodiments, the first or second compound is a dendritic polyol derivative. In some embodiments, the first or second compound is a glycol, trimethylolpropane, glycerol, diglycerol, pentaerythritiol, sorbitol, hexaglycerol, tripentaerythritol, or polyglycerol derivative. In certain embodiments, the first or second compound is a glycol, trimethylolpropane, pentaerythritol, hexaglycerol, or tripentaerythritol derivative. In some embodiments, the first or second compound is a trimethylolpropane, glycerol, diglycerol, pentaerythritiol, sorbitol, hexaglycerol, tripentaerythritol, or polyglycerol derivative. In some embodiments, the first or second compound is a pentaerythritol, di-pentaerythritol, or tri-pentaerythritol derivative. In certain embodiments, the first or second compound is a hexaglycerol (2-ethyl-2-(hydroxymethyl)-1,3-propanediol, trimethylolpropane) derivative. In some embodiments, the first or second compound is a sorbitol derivative. In certain embodiments, the first or second compound is a glycol, propyleneglycol, glycerin, diglycerin, or polyglycerin derivative.

In some embodiments, the first and/or second compound comprise polyethylene glycol (PEG) chains comprising one to 200 ethylene glycol subunits. In certain embodiments, the first and/or second compound may further comprise polypropylene glycol (PPG) chains comprising one to 200 propylene glycol subunits. The PEG or PPG chains extending from the polyols are the “arms” linking the polyol core to the nucleophilic or electrophilic groups.

### Exemplary Nucleophilic Monomers

The biocompatible pre-formulation comprises at least one first compound comprising more than one nucleophilic group. In some embodiments, the first compound is a monomer configured to form a polymer matrix through the reaction of a nucleophilic group in the first compound with an electrophilic group of a second compound. In some embodiments, the first compound monomer is fully synthetic. In some embodiments, the nucleophilic group is a hydroxyl, thiol, or amino group. In preferred embodiments, the nucleophilic group is a thiol or amino group. In some embodiments, the at least one first compound is glycol-based. In some embodiments, glycol-based compounds include ethylene glycol, propylene glycol, butylene glycol, alkyl glycols of various chain lengths, and any combination or copolymers thereof. In some embodiments, glycol-based compounds are polyglycol-based compounds. In some embodiments, the polyglycol-based compounds include, but are not limited to, polyethylene glycols (PEGs), polypropylene glycols (PPGs), polybutylene glycols (PBGs), and polyglycol copolymers. In some embodiments, glycol-based compounds include polyethylene glycol, polypropylene glycol, polybutylene glycol, polyalkyl glycols of various chain lengths, and any combination or copolymers thereof. In some embodiments, the glycol-based compounds are fully synthetic. In some embodiments, the polyglycol-based compounds are fully synthetic.

In certain embodiments, the nucleophilic group is connected to the polyol derivative through a suitable linker. Suitable linkers include, but are not limited to, esters (e.g., acetates) or ethers. In some instances, monomers comprising ester linkers are more susceptible to biodegradation. Examples of linkers comprising a nucleophilic group include, but are not limited to, mercaptoacetate, aminoacetate (glycin) and other amino acid esters (e.g., alanine, β-alanine, lysine, ornithine), 3-mercaptopropionate, ethylamine ether, or propylamine ether. In some embodiments, the polyol core derivative is bound to a polyethylene glycol or polypropylene glycol subunit, which is connected to the linker comprising the nucleophilic group. The molecular weight of the first compound (the nucleophilic monomer) is about 500 to 40000. In certain embodiments, the molecular weight of a first compound (a nucleophilic monomer) is about 100, about 500, about 1000, about 2000, about 3000, about 4000, about 5000, about 6000, about 7000, about 8000, about 9000, about 10000, about 12000, about 15000, about 20000, about 25000, about 30000, about 35000, about 40000, about 50000, about 60000, about 70000, about 80000, about 90000, or about 100000. In some embodiments, the molecular weight of a first compound is about 500 to 2000. In certain embodiments, the molecular weight of a first compound is about 15000 to about 40000. In some embodiments, the first compound is water soluble.

In some embodiments, the first compound is a MULTIARM-(5k-50k)-polyol derivative comprising polyglycol subunits and more than two nucleophilic groups. MULTIARM refers to number of polyglycol subunits that are attached to the polyol core and these polyglycol subunits link the nucleophilic groups to the polyol core. In some embodiments, MULTIARM is 3ARM, 4ARM, 6ARM, 8ARM, 10ARM, 12ARM. In some embodiments, MULTIARM is 4ARM or 8ARM. In some embodiments, the first compound is MULTIARM-(5k-50k)-NH2, MULTIARM-(5k-50k)-AA, or a combination thereof. In certain embodiments, the first compound is 4ARM-(5k-50k)-NH2, 4ARM-(5k-50k)-AA, 8ARM-(5k-50k)-NH2, and 8ARM-(5k-50k)-AA, or a combination thereof. In some embodiments, the polyol derivative is a glycol, trimethylolpropane, glycerol, diglycerol, pentaerythritol, sorbitol, hexaglycerol, tripentaerythritol, or polyglycerol derivative.

Examples of the construction of monomers comprising more than one nucleophilic group are shown below with a trimethylolpropane or pentaerythritol core polyol. The compounds shown have thiol or amine electrophilic groups that are connected to variable lengths PEG subunit through acetate, propionate or ethyl ether linkers (e.g., structures below of ETTMP (A; n=1), 4ARM-PEG-NH2 (B; n=1), and 4ARM-PEG-AA (C; n=1)). Monomers using other polyol cores are constructed in a similar way.

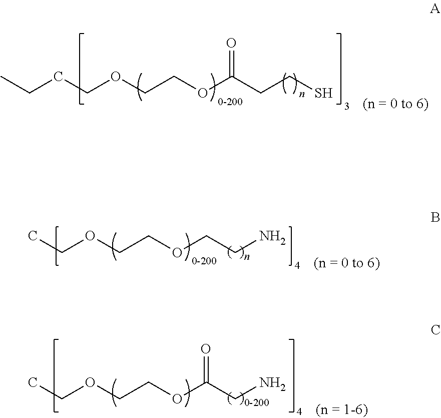

Suitable first compounds comprising a nucleophilic group (used in the amine-ester chemistry) include, but are not limited to, pentaerythritol polyethylene glycol amine (4ARM-PEG-NH2) (molecular weight selected from about 5000 to about 40000, e.g., 5000, 10000, or 20000), pentaerythritol polyethylene glycol amino acetate (4ARM-PEG-AA) (molecular weight selected from about 5000 to about 40000, e.g., 5000, 10000, or 20000), hexaglycerin polyethylene glycol amine (8ARM-PEG-NH2) (molecular weight selected from about 5000 to about 40000, e.g., 10000, 20000, or 40000), or tripentaerythritol glycol amine (8ARM(TP)-PEG-NH2) (molecular weight selected from about 5000 to about 40000, e.g., 10000, 20000, or 40000). Within this class of compounds, 4(or 8)ARM-PEG-AA comprises ester (or acetate) groups while the 4(or 8)ARM-PEG-NH2 monomers do not comprise ester (or acetate) groups.

Other suitable first compounds comprising a nucleophilic group (used in the thiol-ester chemistry) include, but not limited to, glycol dimercaptoacetate (THIOCURE® GDMA), trimethylolpropane trimercaptoacetate (THIOCURE® TMPMA), pentaerythritol tetramercaptoacetate (THIOCURE® PETMA), glycol di-3-mercaptopropionate (THIOCURE® GDMP), trimethylolpropane tri-3-mercaptopropionate (THIOCURE® TMPMP), pentaerythritol tetra-3-mercaptopropionate (THIOCURE® PETMP), polyol-3-mercaptopropionates, polyester-3-mercaptopropionates, propyleneglycol 3-mercaptopropionate (THIOCURE® PPGMP 800), propyleneglycol 3-mercaptopropionate (THIOCURE® PPGMP 2200), ethoxylated trimethylolpropane tri-3-mercaptopropionate (THIOCURE® ETTMP-700), and ethoxylated trimethylolpropane tri-3-mercaptopropionate (THIOCURE® ETTMP-1300).

### Exemplary Electrophilic Monomers

The biocompatible pre-formulation comprises at least one second compound comprising more than one electrophilic group. In some embodiments, the second compound is a monomer configured to form a polymer matrix through the reaction of an electrophilic group in the second compound with a nucleophilic group of a first compound. In some embodiments, the second compound monomer is fully synthetic. In some embodiments, the electrophilic group is an epoxide, maleimide, succinimidyl, or an alpha-beta unsaturated ester. In preferred embodiments, the electrophilic group is an epoxide or succinimidyl. In some embodiments, the at least one second compound is glycol-based. In some embodiments, glycol-based compounds include ethylene glycol, propylene glycol, butylene glycol, alkyl glycols of various chain lengths, and any combination or copolymers thereof. In some embodiments, the glycol-based compound is a polyglycol-based compound. In some embodiments, the polyglycol-based compounds include, but are not limited to, polyethylene glycols (PEGs), polypropylene glycols (PPGs), polybutylene glycols (PBGs), and polyglycol copolymers. In some embodiments, glycol-based compounds include polyethylene glycol, polypropylene glycol, polybutylene glycol, polyalkyl glycols of various chain lengths, and any combination or copolymers thereof. In some embodiments, the glycol-based compounds are fully synthetic. In some embodiments, the polyglycol-based polymer is fully synthetic.

In certain embodiments, the electrophilic group is connected to the polyol derivative through a suitable linker. Suitable linkers include, but are not limited to, esters, amides, or ethers. In some instances, monomers comprising ester linkers are more susceptible to biodegradation. Examples of linkers comprising an electrophilic group include, but are not limited to, succinimidyl succinate, succinimidyl glutarate, succinimidyl succinamide, succinimidyl glutaramide, or glycidyl ether. In some embodiments, the polyol core derivative is bound to a polyethylene glycol or polypropylene glycol subunit, which is connected to the linker comprising the electrophilic group. The molecular weight of the second compound (the electophilic monomer) is about 500 to 40000. In certain embodiments, the molecular weight of a second compound (an electophilic monomer) is about 100, about 500, about 1000, about 2000, about 3000, about 4000, about 5000, about 6000, about 7000, about 8000, about 9000, about 10000, about 12000, about 15000, about 20000, about 25000, about 30000, about 35000, about 40000, about 50000, about 60000, about 70000, about 80000, about 90000, or about 100000. In some embodiments, the molecular weight of a second compound is about 500 to 2000. In certain embodiments, the molecular weight of a second compound is about 15000 to about 40000. In some embodiments, the second compound is water soluble.

In some embodiments, the second compound is a MULTIARM-(5k-50k)-polyol derivative comprising polyglycol subunits and more than two electrophilic groups. MULTIARM refers to number of polyglycol subunits that are attached to the polyol core and these polyglycol subunits link the nucleophilic groups to the polyol core. In some embodiments, MULTIARM is 3ARM, 4ARM, 6ARM, 8ARM, 10ARM, 12ARM or any combination thereof. In some embodiments, MULTIARM is 4ARM or 8ARM. In certain embodiments, the second compound is selected from MULTIARM-(5-50k)-SG, MULTIARM-(5-50k)-SGA, MULTIARM-(5-50k)-SS, MULTIARM-(5-50k)-SSA, and a combination thereof. In certain embodiments, the second compound is selected from 4ARM-(5-50k)-SG, 4ARM-(5-50k)-SGA, 4ARM-(5-50k)-SS, 8ARM-(5-50k)-SG, 8ARM-(5-50k)-SGA and 8ARM-(5-50k)-SS, and a combination thereof. In some embodiments, the polyol derivative is a glycol, trimethylolpropane, glycerol, diglycerol, pentaerythritol, sorbitol, hexaglycerol, tripentaerythritol, or polyglycerol derivative.

Examples of the construction of monomers comprising more than one electrophilic group are shown below with a pentaerythritol core polyol. The compounds shown have a succinimidyl electrophilic group, a glutarate or glutaramide linker, and a variable lengths PEG subunit (e.g., structures below of 4ARM-PEG-SG (D; n=3) and 4ARM-PEG-SGA (E; n=3)). Monomers using other polyol cores or different linkers (e.g., succinate (SS) or succinamide (SSA) are constructed in a similar way.

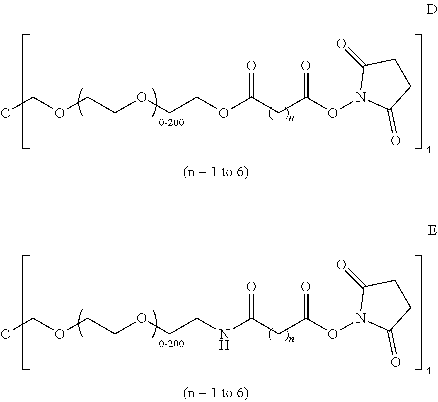

Suitable second compounds comprising an electrophilic group include, but are not limited to, pentaerythritol polyethylene glycol maleimide (4ARM-PEG-MAL) (molecular weight selected from about 5000 to about 40000, e.g., 10000 or 20000), pentaerythritol polyethylene glycol succinimidyl succinate (4ARM-PEG-SS) (molecular weight selected from about 5000 to about 40000, e.g., 10000 or 20000), pentaerythritol polyethylene glycol succinimidyl glutarate (4ARM-PEG-SG) (molecular weight selected from about 5000 to about 40000, e.g., 10000 or 20000), pentaerythritol polyethylene glycol succinimidyl glutaramide (4ARM-PEG-SGA) (molecular weight selected from about 5000 to about 40000, e.g., 10000 or 20000), hexaglycerin polyethylene glycol succinimidyl succinate (8ARM-PEG-SS) (molecular weight selected from about 5000 to about 40000, e.g., 10000 or 20000), hexaglycerin polyethylene glycol succinimidyl glutarate (8ARM-PEG-SG) (molecular weight selected from about 5000 to about 40000, e.g., 10000, 15000, 20000, or 40000), hexaglycerin polyethylene glycol succinimidyl glutaramide (8ARM-PEG-SGA) (molecular weight selected from about 5000 to about 40000, e.g., 10000, 15000, 20000, or 40000), tripentaerythritol polyethylene glycol succinimidyl succinate (8ARM(TP)-PEG-SS) (molecular weight selected from about 5000 to about 40000, e.g., 10000 or 20000), tripentaerythritol polyethylene glycol succinimidyl glutarate (8ARM(TP)-PEG-SG) (molecular weight selected from about 5000 to about 40000, e.g., 10000, 15000, 20000, or 40000), or tripentaerythritol polyethylene glycol succinimidyl glutaramide (8ARM(TP)-PEG-SGA) (molecular weight selected from about 5000 to about 40000, e.g., 10000, 15000, 20000, or 40000). The 4(or 8)ARM-PEG-SG monomers comprise ester groups, while the 4(or 8)ARM-PEG-SGA monomers do not comprise ester groups.

Other suitable second compounds comprising an electrophilic group are sorbitol polyglycidyl ethers, including, but not limited to, sorbitol polyglycidyl ether (DENACOL® EX-611), sorbitol polyglycidyl ether (DENACOL® EX-612), sorbitol polyglycidyl ether (DENACOL® EX-614), sorbitol polyglycidyl ether (DENACOL® EX-614 B), polyglycerol polyglycidyl ether (DENACOL® EX-512), polyglycerol polyglycidyl ether (DENACOL® EX-521), diglycerol polyglycidyl ether (DENACOL® EX-421), glycerol polyglycidyl ether (DENACOL® EX-313), glycerol polyglycidyl ether (DENACOL® EX-313), trimethylolpropane polyglycidyl ether (DENACOL® EX-321), sorbitol polyglycidyl ether (DENACOL® EJ-190).

### Formation of Biocompatible Hydrogel Polymer Matrices

Provided herein are biocompatible pre-formulations, comprising at least one first compound comprising more than one nucleophilic group, at least one second compound comprising more than one electrophilic group, optionally at least one cell, and optionally additional components. An exemplary additional component is a culture medium. In certain embodiments, the culture medium is a buffer. In certain embodiments, the culture medium is a nutrient rich medium. In certain embodiments the cell is a stem cell. The biocompatible pre-formulation undergoes polymerization and/or gelling to form a biocompatible hydrogel polymer matrix. In certain embodiments, the biocompatible hydrogel polymer matrix is biodegradable. In certain embodiments, the biocompatible hydrogel polymer matrix comprises a biocompatible hydrogel scaffold.

Provided herein are biocompatible pre-formulations, comprising at least one first compound comprising more than one nucleophilic group, at least one second compound comprising more than one electrophilic group, a culture medium, and optionally additional components. An exemplary additional component is at least one cell. In certain embodiments the cell is a stem cell. In certain embodiments, the culture medium is a buffer. In certain embodiments, the culture medium is a nutrient rich medium. The biocompatible pre-formulation undergoes polymerization and/or gelling to form a biocompatible hydrogel polymer matrix. In certain embodiments, the biocompatible hydrogel polymer matrix is biodegradable. In certain embodiments, the biocompatible hydrogel polymer matrix comprises a biocompatible hydrogel scaffold.

In certain embodiments, the pre-formulation safely undergoes polymerization at a target site inside or on a mammalian body, for instance at the site of a wound, surgical site, or in a joint. In certain embodiments, the biocompatible hydrogel polymer matrix forms a wound patch, suture, or joint spacer. In some embodiments, the first compound and the second compound are monomers forming a polymer matrix through the reaction of a nucleophilic group in the first compound with the electrophilic group in the second compound. In certain embodiments, the monomers are polymerized at a predetermined time. In some embodiments, the monomers are polymerized under mild and nearly neutral pH conditions. In certain embodiments, the biocompatible hydrogel polymer matrix does not change volume after gelling.

In some embodiments, the first and second compounds react to form amide, thioester, or thioether bonds. When a thiol nucleophile reacts with a succinimidyl electrophile, a thioester is formed. When an amino nucleophile reacts with a succinimidyl electrophile, an amide is formed.

In some embodiments, one or more first compounds comprising an amino group react with one or more second compounds comprising a succinimidyl ester group to form amide linked first and second monomer units. In certain embodiments, one or more first compounds comprising a thiol group react with one or more second compounds comprising a succinimidyl ester group to form thioester linked first and second monomer units. In some embodiments, one or more first compounds comprising an amino group react with one or more second compounds comprising an epoxide group to from amine linked first and second monomer units. In certain embodiments, one or more first compounds comprising a thiol group react with one or more second compounds comprising an epoxide group to form thioether linked first and second monomer units.

In some embodiments, a first compound is mixed with a different first compound before addition to one or more second compounds. In other embodiments, a second compound is mixed with a different second compound before addition to one or more first compounds. In certain embodiments, the properties of the biocompatible pre-formulation and the biocompatible hydrogel polymer matrix are controlled by the properties of the at least one first and at least one second monomer mixture.

In some embodiments, one first compound is used in the biocompatible hydrogel polymer matrix. In certain embodiments, two different first compounds are mixed and used in the biocompatible hydrogel polymer matrix. In some embodiments, three different first compounds are mixed and used in the biocompatible hydrogel polymer matrix. In certain embodiments, four or more different first compounds are mixed and used in the biocompatible hydrogel polymer matrix.

In some embodiments, one second compound is used in the biocompatible hydrogel polymer matrix. In certain embodiments, two different second compounds are mixed and used in the biocompatible hydrogel polymer matrix. In some embodiments, three different second compounds are mixed and used in the biocompatible hydrogel polymer matrix. In certain embodiments, four or more different second compounds are mixed and used in the biocompatible hydrogel polymer matrix.

In some embodiments, a first compound comprising ether linkages to the nucleophilic group are mixed with a different first compound comprising ester linkages to the nucleophilic group. This allows the control of the concentration of ester groups in the resulting biocompatible hydrogel polymer matrix. In certain embodiments, a second compound comprising ester linkages to the electrophilic group are mixed with a different second compound comprising ether linkages to the electrophilic group. In some embodiments, a second compound comprising ester linkages to the electrophilic group are mixed with a different second compound comprising amide linkages to the electrophilic group. In certain embodiments, a second compound comprising amide linkages to the electrophilic group are mixed with a different second compound comprising ether linkages to the electrophilic group.

In some embodiments, a first compound comprising an aminoacetate (e.g., glycine derived) nucleophile is mixed with a different first compound comprising an amine nucleophile (e.g., an ethylamine ether) at a specified molar ratio (x/y). In certain embodiments, the molar ratio (x/y) is 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In certain embodiments, a first compound comprising an aminoacetate (e.g., glycine derived) nucleophile is mixed with a different first compound comprising an amine nucleophile (e.g., an ethylamine ether) at a specified weight ratio (x/y). In certain embodiments, the weight ratio (x/y) is 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In certain embodiments, the mixture of two first compounds is mixed with one or more second compounds at a molar amount equivalent to the sum of x and y.

In some embodiments, the first compound comprising more than one nucleophilic group and the optional at least one cell are pre-mixed in the presence of water. In some embodiments, the first compound comprising more than one nucleophilic group and the cell are pre-mixed without the presence of water. Once pre-mixing is complete, the second compound comprising more than one electrophilic group is added to the pre-mixture in the presence of water to form a biocompatible hydrogel polymer matrix. Shortly after final mixing, the biocompatible hydrogel polymer matrix mixture is delivered to the target site. In certain embodiments, an optional additional component is added to the pre-mix, the second compound, or to the mixture just before delivery of the biocompatible hydrogel polymer matrix mixture to the target site. In certain embodiments, an optional additional component is added to the pre-mix, the second compound, or to the mixture after delivery of the biocompatible hydrogel polymer matrix mixture to the target site. In some embodiments, the additional component is a buffer. In some embodiments, the biocompatible hydrogel polymer matrix polymerizes and/or gels prior to delivery to the target site. In some embodiments, the biocompatible hydrogel polymer matrix polymerizes and/or gels at the target site.

In some embodiments, the first compound comprising more than one nucleophilic group and the buffer are pre-mixed in the presence of water. In some embodiments, the first compound comprising more than one nucleophilic group and the buffer are pre-mixed without the presence of water. Once pre-mixing is complete, the second compound comprising more than one electrophilic group is added to the pre-mixture in the presence of water, forming a biocompatible hydrogel polymer matrix. Shortly after final mixing, the biocompatible hydrogel polymer matrix mixture is delivered to the target site. In certain embodiments, an optional additional component is added to the pre-mix, the second compound, or to the mixture just before delivery of the biocompatible hydrogel polymer matrix mixture to the target site. In certain embodiments, an optional additional component is added to the pre-mix, the second compound, or to the mixture after delivery of the biocompatible hydrogel polymer matrix mixture to the target site. In some embodiments, the additional component is at least one cell. In some embodiments, the biocompatible hydrogel polymer matrix polymerizes and/or gels prior to delivery to the target site. In some embodiments, the biocompatible hydrogel polymer matrix polymerizes and/or gels at the target site.

In other embodiments, the second compound comprising more than one electrophilic group and the optional at least one cell are pre-mixed in the presence of water. In other embodiments, the second compound comprising more than one electrophilic group and the cell are pre-mixed without the presence of water. Once pre-mixing is complete, the first compound comprising more than one nucleophilic group is added to the pre-mixture, forming a biocompatible hydrogel polymer matrix. Shortly after final mixing, the biocompatible hydrogel polymer matrix mixture is delivered to the target site. In certain embodiments, an optional component is added to the pre-mix, the first compound, or to the mixture just before delivery of the biocompatible hydrogel polymer matrix mixture to the target site. In certain embodiments, an optional additional component is added to the pre-mix, the first compound, or to the mixture after delivery of the biocompatible hydrogel polymer matrix mixture to the target site. In some embodiments, the additional component is a buffer. In some embodiments, the biocompatible hydrogel polymer matrix polymerizes and/or gels prior to delivery to the target site. In some embodiments, the biocompatible hydrogel polymer matrix polymerizes and/or gels at the target site.

In other embodiments, the second compound comprising more than one electrophilic group and the buffer are pre-mixed in the presence of water. In other embodiments, the second compound comprising more than one electrophilic group and the buffer are pre-mixed without the presence of water. Once pre-mixing is complete, the first compound comprising more than one nucleophilic group is added to the pre-mixture, forming a biocompatible hydrogel polymer matrix. Shortly after final mixing, the biocompatible hydrogel polymer matrix mixture is delivered to the target site. In certain embodiments, an optional component is added to the pre-mix, the first compound, or to the mixture just before delivery of the biocompatible hydrogel polymer matrix mixture to the target site. In certain embodiments, an optional additional component is added to the pre-mix, the first compound, or to the mixture after delivery of the biocompatible hydrogel polymer matrix mixture to the target site. In some embodiments, the additional component is at least one cell. In some embodiments, the biocompatible hydrogel polymer matrix polymerizes and/or gels prior to delivery to the target site. In some embodiments, the biocompatible hydrogel polymer matrix polymerizes and/or gels at the target site.

In some embodiments, a first compound comprising more than one nucleophilic group, a second compound comprising more than one electrophilic group, and at least one cell are mixed together in the presence of water, whereby a biocompatible hydrogel polymer matrix is formed. In some embodiments, a first compound comprising more than one nucleophilic group, a second compound comprising more than one electrophilic group, and a buffer are mixed together in the presence of water, whereby a biocompatible hydrogel polymer matrix is formed. In some embodiments, a first compound comprising more than one nucleophilic group, a second compound comprising more than one electrophilic group, optionally at least one cell, and a buffer are mixed together in the presence of water, whereby a biocompatible hydrogel polymer matrix is formed. In certain embodiments, the first compound comprising more than one nucleophilic group, the second compound comprising more than one electrophilic group, and/or the cell are individually diluted in an aqueous buffer in the pH range of about 5.0 to about 9.5, wherein the individual dilutions or neat monomers are mixed and a biocompatible hydrogel polymer matrix is formed. In some embodiments, the aqueous buffer is in the pH range of about 6.0 to about 8.5. In certain embodiments, the aqueous buffer is in the pH range of about 8. In certain embodiments, the aqueous buffer is a culture medium. In certain embodiments, the culture medium is a nutrient rich medium.

In certain embodiments, the concentration of the monomers in the aqueous is from about 1% to about 100%. In some embodiments, the dilution is used to adjust the viscosity of the monomer dilution. In certain embodiments, the concentration of a monomer in the aqueous buffer is about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the electrophilic and nucleophilic monomers are mixed in such ratio that there is a slight excess of electrophilic groups present in the mixture. In certain embodiments, this excess is about 10%, about 5%, about 2%, about 1%, about 0.9%, about 0.8%, about 0.7%, about 0.6%, about 0.5%, about 0.4%, about 0.3%, about 0.2%, about 0.1%, or less than 0.1%.

In certain embodiments, the gelling time or curing time of the biocompatible hydrogel polymer matrix is controlled by the selection of the first and second compounds. In some embodiments, the concentration of nucleophilic or electrophilic groups in the first or second compound influences the gelling time of the biocompatible pre-formulation. In certain embodiments, temperature influences the gelling time of the biocompatible pre-formulation. In some embodiments, the type of aqueous buffer influences the gelling time of the biocompatible pre-formulation. In some embodiments, the aqueous buffer is a culture medium. In certain embodiments, the concentration of the aqueous buffer influences the gelling time of the biocompatible pre-formulation. In some embodiments, the nucleophilicity and/or electrophilicity of the nucleophilic and electrophilic groups of the monomers influences the gelling time of the biocompatible pre-formulation. In some embodiments, the cell type influences the gelling time of the biocompatible pre-formulation. In some embodiments, the cell concentration influences the gelling time of the biocompatible pre-formulation.

In some embodiments, the gelling time or curing time of the biocompatible hydrogel polymer matrix is controlled by the pH of the aqueous buffer. In certain embodiments, the gelling time is between about 20 seconds and 10 minutes. In some embodiments, the gelling time is less than 30 minutes, less than 20 minutes, less than 10 minutes, less than 5 minutes, less than 4.8 minutes, less than 4.6 minutes, less than 4.4 minutes, less than 4.2 minutes, less than 4.0 minutes, less than 3.8 minutes, less than 3.6 minutes, less than 3.4 minutes, less than 3.2 minutes, less than 3.0 minutes, less than 2.8 minutes, less than 2.6 minutes, less than 2.4 minutes, less than 2.2 minutes, less than 2.0 minutes, less than 1.8 minutes, less than 1.6 minutes, less than 1.4 minutes, less than 1.2 minutes, less than 1.0 minutes, less than 0.8 minutes, less than 0.6 minutes, or less than 0.4 minutes. In certain embodiments, the pH of the aqueous buffer is from about 5 to about 9.5. In some embodiments, the pH of the aqueous buffer is from about 7.0 to about 9.5. In specific embodiments, the pH of the aqueous buffer is about 8. In some embodiments, the pH of the aqueous buffer is about 5, about 5.5, about 6.0, about 6.5, about 6.6, about 6.7, about 6.8, about 6.9, about 7.0, about 7.1, about 7.2, about 7.3, about 7.4, about 7.5, about 7.6, about 7.8, about 7.9, about 8.0, about 8.1 about 8.2 about 8.3, about 8.4, about 8.5, about 9.0, or about 9.5.

In certain embodiments, the gelling time or curing time of the biocompatible pre-formulation is controlled by the type of aqueous buffer. In some embodiments, the aqueous buffer is a physiologically acceptable buffer. In certain embodiments, aqueous buffers include, but are not limited to, aqueous saline solutions, phosphate buffered saline, borate buffered saline, a combination of borate and phosphate buffers wherein each component is dissolved in separate buffers, N-2-Hydroxyethylpiperazine-N′-2-hydroxypropanesulfonic acid (HEPES), 3-(N-Morpholino) propanesulfonic acid (MOPS), 2-([2-Hydroxy-1,1-bis(hydroxymethyl)ethyl]amino)ethanesulfonic acid (TES), 3-[N-tris(Hydroxy-methyl) ethylamino]-2-hydroxyethyl]-1-piperazinepropanesulfonic acid (EPPS), Tris[hydroxymethyl]-aminomethane (THAM), and Tris[hydroxymethyl]methyl aminomethane (TRIS). In some embodiments, the thiol-ester chemistry (e.g., ETTMP nucleophile with SGA or SG electrophile) is performed in borate buffer. In certain embodiments, the amine-ester chemistry (NH2 or AA nucleophile with SGA or SG electrophile) is performed in phosphate buffer. In some embodiments the aqueous buffer is a culture medium. In certain embodiments, culture media include, but are not limited to, DMEM, IMDM, OptiMEM®, AlgiMatrix™, Fetal Bovine Serum, GS1-R®, G52-M®, iSTEM®, NDiff® N2,NDiff® N2-AF, RHB-A®, RHB-Basal®, RPMI, SensiCell™, GlutaMAX™, FluoroBrite™, Gibco® TAP, Gibco® BG-11, LB, M9 Minimal, Terrific Broth, 2YXT, MagicMedia™, ImMedia™, SOC, YPD, CSM, YNB, Grace's Insect Media, 199/109 and HamF10/HamF12. In certain embodiments, the cell culture medium may be serum free. In certain embodiments, the culture media may include additives. In some embodiments, culture media additives include, but are not limited to, antibiotics, vitamins, proteins, inhibitors, small molecules, minerals, inorganic salts, nitrogen, growth factors, amino acids, serum, carbohydrates, lipids, hormones and glucose. In some embodiments, growth factors include, but are not limited to, EGF, bFGF, FGF, ECGF, IGF-1, PDGF, NGF, TGF-α and TGF-β. In certain embodiments, the culture medium may not be aqueous. In certain embodiments, the non-aqueous culture media include, but are not limited to, frozen cell stocks, lyophilized medium, and agar.

In certain embodiments, the biocompatible hydrogel polymer matrix comprises a biocompatible hydrogel scaffold. In certain embodiments, the biocompatible hydrogel scaffold comprises the pre-formulation at least one first compound and the pre-formulation at least one second compound. In certain embodiments, the biocompatible hydrogel scaffold comprises a buffer. In certain embodiments, the biocompatible hydrogel scaffold is fully synthetic. In certain embodiments, the biocompatible hydrogel scaffold provides an environment suitable for sustained cell viability and/or growth.

In certain embodiments, the first compound and the second compound do not react with the cell during formation of the biocompatible hydrogel polymer matrix. In some embodiments, the cell remains unchanged after polymerization of the first and second compounds (i.e., monomers). In certain embodiments, the cell, if included, does not change the properties of the biocompatible hydrogel polymer matrix. In some embodiments, the physiochemical properties of the cell and the biocompatible hydrogel polymer matrix formulation are not affected by the polymerization of the monomers. In certain embodiments, delivery of the cell using a biocompatible hydrogel polymer matrix minimizes the degradation or denaturing of the cell. In some instances, the physiochemical properties of the cell are not affected by the delivery or release of the cell to the target site.

In some embodiments, the biocompatible hydrogel polymer matrix formulations further comprise a contrast agent for visualizing the biocompatible hydrogel polymer matrix formulation and locating a tumor using e.g., X-ray, fluoroscopy, or computed tomography (CT) imaging. In certain embodiments, the contrast agent enables the visualization of the bioabsorption of the biocompatible hydrogel polymer matrix. In some embodiments, the contrast agent is a radiopaque material. In certain embodiments, the radiopaque material is selected from, but not limited to, sodium iodide, potassium iodide, and barium sulfate, VISIPAQUE®, OMNIPAQUE®, or HYPAQUE®, tantalum, and similar commercially available compounds, or combinations thereof. In other embodiments, the biocompatible hydrogel polymer matrix further comprises a pharmaceutically acceptable dye.

In some embodiments, the biocompatible hydrogel polymer matrix formulations further comprise a viscosity enhancer. Examples of viscosity enhancer include, but are not limited to, hydroxyethylcellulose, hydroxypropylcellulose, methylcellulose, polyvinylcellulose, polyvinylpyrrolidone.

In some embodiments, they hydrogel is from a composition comprising:
(a) at least one multifunctional nucleophilic monomer comprising a polyol core, wherein the polyol core is selected from the group consisting of:

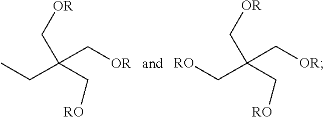

and
   wherein R is:

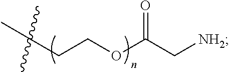

wherein n is 10-200 and the molecular weight of the nucleophilic monomer is between about 2,000 to about 40,000;
(b) at least one water soluble second compound comprising more than one electrophilic group selected from an epoxide, N-succinimidyl succinate, N-succinimidyl glutarate, N-succinimidyl succinamide or N-succinimidyl glutaramide, wherein the second compound comprising the core of one electrophilic group is a pentaerythritol, and wherein the second compound further comprises one or more polyethylene glycol sections; and
(c) an aqueous buffer in the pH range of 5.0 to 9.0.

In some embodiments, the hydrogel is formed when the compositions comprising the at least one multifunctional nucleophilic monomer and the at least one water soluble second compound in the aqueous buffer is mixed and applied (placed) at a target site in or on the subject. In some embodiments, the hydrogel does not comprise blood, protein, or other contaminants.

In some embodiments, the molecular weight of each of the second compound is independently between about 500 and 40000. In some embodiments, the second compound is selected from the group consisting of ethoxylated pentaerythritol succinimidyl succinate, ethoxylated pentaerythritol succinimidyl glutarate, and ethoxylated pentaerythritol succinimidyl glutaramide. In some embodiments, the composition, further comprises a therapeutic agent selected from the group consisting of an anticancer agent, an antiviral agent, an antibacterial agent, antifungal agent, an immunosuppressant agent, an hemo stasis agent, and an anti-inflammatory agent. In some embodiments, the agent is silver. In some embodiments, the pH of the aqueous buffer is from about 6.9 to about 7.9. In some embodiments, the biocompatible hydrogel polymer is bioabsorbed within about 1 to 70 days. In some embodiments, the biocompatible hydrogel polymer is substantially non-bioabsorbable.

In some embodiments, the composition further comprises a second multifunctional nucleophilic monomer comprising more than one nucleophilic group, wherein the second multifunctional nucleophilic monomer is a polyol substituted with R′, wherein R′ is:

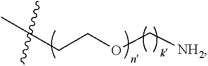

wherein n′ is 1-200, and

wherein k′ is 1-6.

In some embodiments, the polyol core of the multifunctional nucleophilic monomer is:

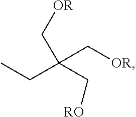

wherein R is:

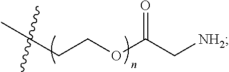

wherein n is 10-200 and the molecular weight of the nucleophilic monomer is between about 2,000 to about 40,000. In some embodiments,

In some embodiments, the molecular weight of the multifunctional nucleophilic monomer is between about 5,000 to about 20,000.

In some embodiments, the mixture comprising at least one multifunctional nucleophilic monomer and the at least one water soluble second compound comprising more than one electrophilic group comprises 4ARM-20k-AA and 4ARM-20k-SGA.

In some embodiments, the hydrogel that can be applied to a target site and treated with a laser comprises a polymer prepared from monomers consisting of: (a) 8-ARM-20k-NH2 PEG amine, 4-ARM-20k-AA acetate amine, and 8-ARM-PEG-SG monomer; or (b) 8-ARM-20k-NH2 PEG amine, 8-ARM-20k-AA acetate amine, and 8-ARM-PEG-SG monomer and wherein the biocompatible hydrogel polymer dos not contain blood or protein

In some embodiments, the hydrogel is prepared from a composition comprising:
(a) one or more multi-ARM nucleophilic PEG monomers, wherein the multi-ARM PEG nucleophilic monomers comprise a polyol core, wherein the polyol core is selected from the group consisting of

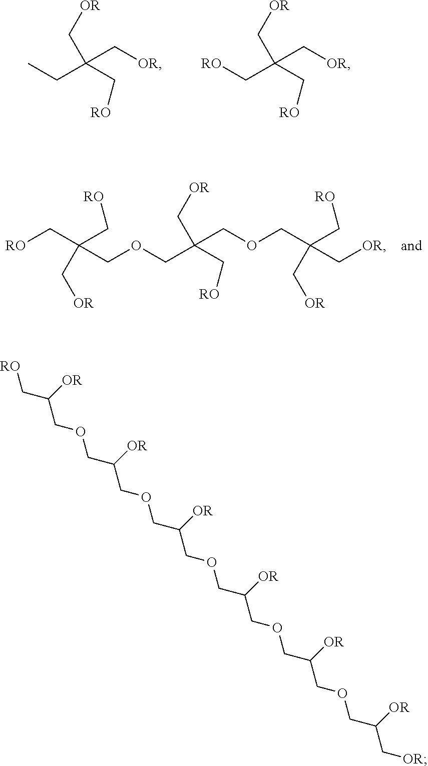

wherein the polyol core is substituted with 3-8 R-groups, wherein R is:

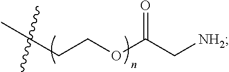

wherein n is 1-200;
(b) one or more multi-ARM nucleophilic PEG monomers, wherein the multi-ARM PEG nucleophilic monomers comprise a polyol core, wherein the polyol core is selected from the group consisting of

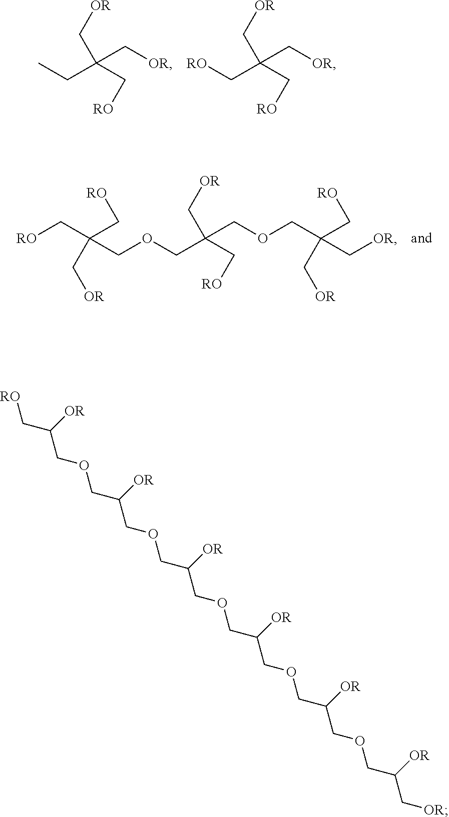

wherein the polyol core is substituted with 3-8 R-groups, wherein R is:

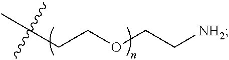

wherein n is 1-200;
(c) one or more multi-ARM-PEG electrophilic monomers having more than two electrophilic arms, wherein each electrophilic arm comprises a PEG chain and terminates in an electrophilic group; and
(d) an aqueous buffer in the pH range of about 5.0 to about 9.5;
wherein the molecular weight of the multi-ARM PEG nucleophilic monomers and/or the multi-ARM PEG electrophilic monomers is about 500 to about 40000.

In some embodiments, the molecular weight of the multi-ARM PEG nucleophilic monomers and/or the multi-ARM PEG electrophilic monomers is about 15000 to about 40000.

In some embodiments, the hydrogel is prepared by mixing:
(a) one or more multi-ARM nucleophilic PEG monomers, wherein the multi-ARM PEG nucleophilic monomers comprise a polyol core, wherein the polyol core is selected from the group consisting of

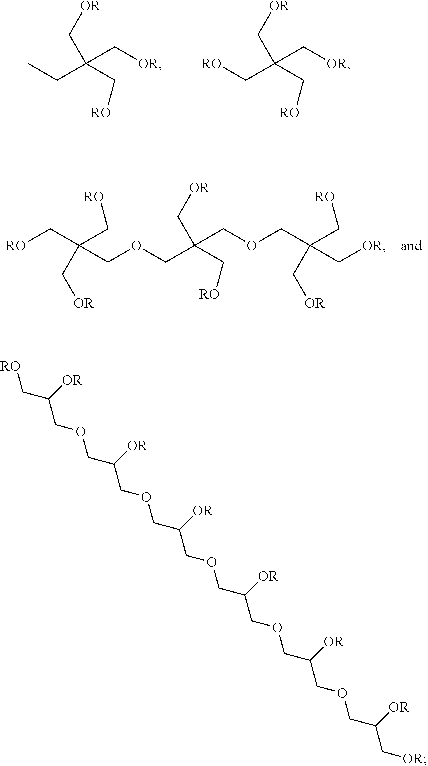

wherein the polyol core is substituted with 3-8 R-groups, wherein R is:

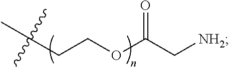

wherein n is 1-200;
(b) one or more multi-ARM nucleophilic PEG monomers, wherein the multi-ARM PEG nucleophilic monomers comprise a polyol core, wherein the polyol core is selected from the group consisting of

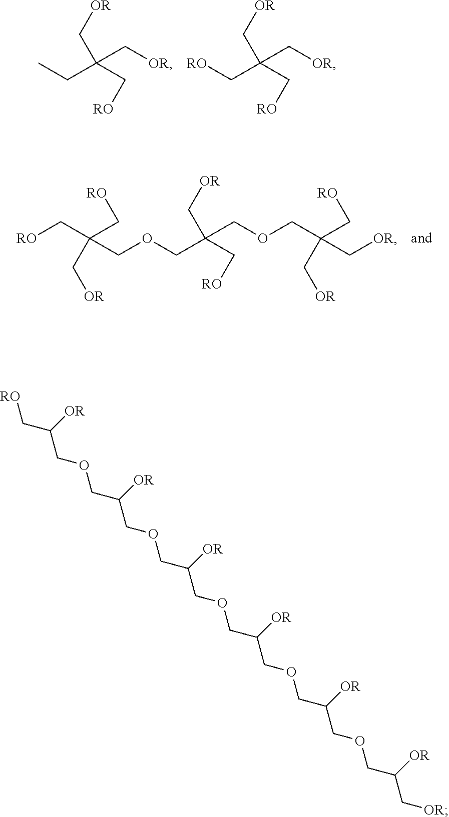

wherein the polyol core is substituted with 3-8 R-groups, wherein R is:

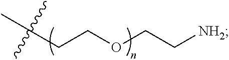

wherein n is 1-200;
(c) one or more multi-ARM-PEG electrophilic monomers having more than two electrophilic arms, wherein each electrophilic arm comprises a PEG chain and terminates in an electrophilic group; and
(d) an aqueous buffer in the pH range of about 5.0 to about 9.5,
wherein the molecular weight of the multi-ARM PEG nucleophilic monomers and/or the multi-ARM PEG electrophilic monomers is about 500 to about 40000.

In some embodiments, the mixing is performed before it is applied to a target site on the subject. In some embodiments, the hydrogel gels or polymerizes at least in part at the target site. In some embodiments, the hydrogel gels or polymerizes completely before being applied to a target site.

In some embodiments, the molecular weight of the multi-ARM PEG nucleophilic monomers and/or the multi-ARM PEG electrophilic monomers is about 15000 to about 40000.

In some embodiments, the polyol core of the multi-ARM PEG nucleophilic monomer is:

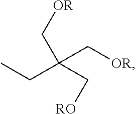

wherein R is

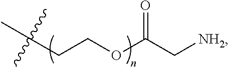

wherein n is 1-200;

In some embodiments, the polyol core of the multi-ARM PEG nucleophilic monomer is:

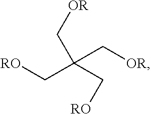

wherein R is

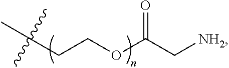

wherein n is 1-200.

In some embodiments, the polyol core of the multi-ARM PEG nucleophilic monomer is:

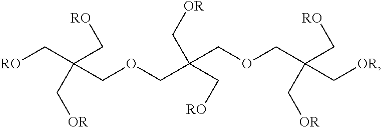

wherein R is

wherein n is 1-200.

In some embodiments, the polyol core of the multi-ARM PEG nucleophilic monomer is:

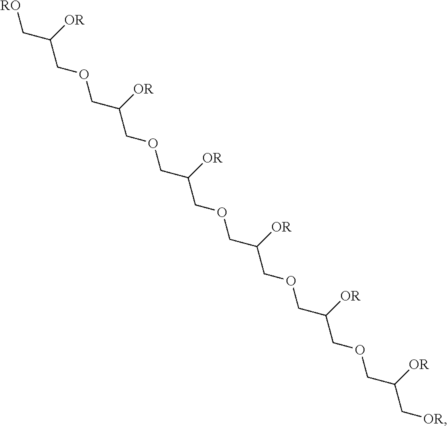

wherein R is

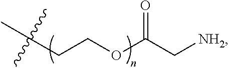

wherein n is 1-200.

In some embodiments, the hydrogel is formed from a composition comprising: (a) at least one solid first compound comprising more than two nucleophilic groups; (b) at least one solid second compound comprising more than two electrophilic groups; (c) optionally, a solid buffer component; (d) optionally, a therapeutic agent, which may be solid; and (e) optionally, a solid viscosity enhancer wherein the solid polyglycol-based, fully synthetic, pre-formulation polymerizes and/or gels to form a polyglycol-based, fully synthetic, biocompatible hydrogel polymer after addition of a liquid component, wherein the liquid component does not contain any first compound or second compound, and provided that the solid polyglycol-based, fully synthetic, pre-formulation does not contain any aqueous component.

In some embodiments, the liquid component comprises water, saline, a buffer, a therapeutic agent or a combination thereof.

In some embodiments, the nucleophilic group comprises a thiol or amino group.

In some embodiments, the solid first compound is a MULTIARM (5k-50k) polyol derivative comprising polyglycol subunits and more than two nucleophilic groups.

In some embodiments, the electrophilic group comprises an epoxide, N-succinimidyl succinate, N-succinimidyl glutarate, N-succinimidyl succinamide or N-succinimidyl glutaramide.

In some embodiments, the solid second compound is a MULTIARM (5k-50k) polyol derivative comprising polyglycol subunits and more than two electrophilic groups.

In some embodiments, the solid first compound is a MULTIARM-(5-50k)-SH, a MULTIARM-(5-50k)-NH2, a MULTIARM-(5-50k)-AA, or a combination thereof, and the second compound is a MULTIARM-(5-50k)-SG, a MULTIARM-(5-50k)-SGA, a MULTIARM-(5-50k)-SS, or a combination thereof.

In some embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof, and the second compound is 4ARM-10k-SG, 8ARM-15k-SG, 4ARM-20k-SGA, 4ARM-10k-SS, or a combination thereof.

In some embodiments, the solid polyglycol-based pre-formulation of claim 8, wherein the solid first compound is 8ARM-20k-NH2 and/or 8ARM-20k-AA, and the second compound is 4ARM-20k-SGA.

In some embodiments, the therapeutic agent is selected from an antibacterial agent, an antifungal agent, an immunosuppressant agent, an anti-inflammatory agent, a bisphosphonate, gallium nitrate, stem cells, an antiseptic agent, and a lubricity agent.

In some embodiments, the therapeutic agent is a lubricity agent. In some embodiments, the lubricity agent is hyaluronic acid. In some embodiments, the composition is the hydrogel polymer.

In some embodiments, the composition for treating a wound is provided, wherein the composition comprises a hydrogel formed from: (a) at least one solid first compound comprising more than two nucleophilic groups; (b) at least one solid second compound comprising more than two electrophilic groups; (c) optionally, a solid buffer component; (d) optionally, a therapeutic agent (can be solid or not); and (e) optionally, a solid viscosity enhancer, wherein composition polymerizes and/or gels at a target site of the wound to form a hydrogel polymer after addition of a liquid component, wherein the liquid component does not contain any first compound or second compound, and provided that, in some embodiments, the composition does not contain any aqueous component.

In some embodiments, the liquid component comprises water, saline, a buffer, a therapeutic agent, or a combination thereof.

In some embodiments, the solid first compound is a MULTIARM (5k-50k) polyol derivative comprising polyglycol subunits and more than two nucleophilic groups, and wherein the solid second compound is a MULTIARM (5k-50k) polyol derivative comprising polyglycol subunits and more than two electrophilic groups.

In some embodiments, the solid first compound is a MULTIARM-(5-50k)-SH, a MULTIARM-(5-50k)-NH2, a MULTIARM-(5-50k)-AA, or a combination thereof, and the solid second compound is a MULTIARM-(5-50k)-SG, a MULTIARM-(5-50k)-SGA, a MULTIARM-(5-50k)-SS, or a combination thereof.

In some embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof, and the solid second compound is 4ARM-10k-SG, 8ARM-15k-SG, 4ARM-20k-SGA, 4ARM-10k-SS, or a combination thereof.

In some embodiments, the solid first compound is 8ARM-20k-NH2 and/or 8ARM-20k-AA, and the solid second compound is 4ARM-20k-SGA.

In some embodiments, the composition, the hydrogel is formed from a composition comprises:
(a) at least one solid first polyethylene glycol-based compound comprising more than two nucleophilic groups;
(b) at least one solid second polyethylene glycol-based compound comprising more than two electrophilic groups;

wherein the least one solid first polyethylene glycol-based compound is a MULTIARM-(5-50k)-NH2, a MULTIARM-(5-50k)-AA, or a combination thereof;

wherein the at least one solid second polyethylene glycol-based compound is a MULTIARM-(5-50k)-SG, a MULTIARM-(5-50k)-SGA, a MULTIARM-(5-50k)-SS, a MULTIARM-(5-50k)-SSA, or a combination thereof;

wherein the polyglycol-based, fully synthetic, pre-formulation polymerizes and/or gels to form a polyglycol-based, fully synthetic, biocompatible hydrogel polymer after addition of a liquid component;

wherein the liquid component comprises a buffer providing a pH of about 5.0 to about 9.5;

wherein the liquid component does not contain the at least one solid first polyethylene glycol-based compound and the at least one solid second polyethylene glycol-based compound, and provided that the solid polyglycol-based, fully synthetic, pre-formulation does not contain an aqueous component; and wherein the pre-formulation is free of a hemostasis agent.

In some embodiments, the hydrogel is formed by mixing:
(a) at least one solid first polyethylene glycol-based compound comprising more than two nucleophilic groups that does not contain an aqueous component;
(b) at least one solid second polyethylene glycol-based compound comprising more than two electrophilic groups that does not contain an aqueous component;

wherein the least one solid first polyethylene glycol-based compound is a MULTIARM-(5-50k)-NH2, MULTIARM-(5-50k)-AA, or a combination thereof;

wherein the least one solid second polyethylene glycol-based compound is a MULTIARM-(5-50k)-SG, MULTIARM-(5-50k)-SGA, MULTIARM-(5-50k)-SS, MULTIARM-(5-50k)-SSA, or a combination thereof;

wherein the polyglycol-based, fully synthetic, biocompatible hydrogel polymer is formed after addition of a liquid component; wherein the liquid component comprises a buffer providing a pH of about 5.0 to about 9.5,

wherein the liquid component does not contain the at least one solid first polyethylene glycol-based compound or the at least one solid second polyethylene glycol-based compound;

wherein the pre-formulation is free of a hemostasis agent.

In some embodiments, composition polymerizes and/or gels to form a polyglycol-based, fully synthetic, biocompatible hydrogel polymer, which can be at the wound site.

In some embodiments, the first polyethylene glycol-based compound is a MULTIARM-(5-50k)-NH2, a MULTIARM-(5-50k)-AA, or a combination thereof; and wherein the second polyethylene glycol-based compound is a MULTIARM-(5-50k)-SG, a MULTIARM-(5-50k)-SGA, or a combination thereof.

In some embodiments, the MULTIARM of the first polyethylene glycol-based compound and/or the second polyethylene glycol-based compound is 3ARM, 4ARM, 6ARM, or 8ARM. In some embodiments, the first polyethylene glycol-based compound is 4ARM-2k-NH2, 4ARM-5k-NH2, 8ARM-20k-NH2, 4ARM-20k-AA, 8ARM-20k-AA, or a combination thereof, and the second polyethylene glycol-based compound is 4ARM-10k-SG, 8ARM-15k-SG, 4ARM-20k-SGA, 4ARM-10k-SS, or a combination thereof.

In some embodiments, the first polyethylene glycol-based compound is 8ARM-20k-NH2 and/or 8ARM-20k-AA and the second polyethylene glycol-based compound is 4ARM-20k-SGA.

In some embodiments, the first polyethylene glycol-based compound is a MULTIARM-(5-50k)-NH2, a MULTIARM-(5-50k)-AA, or a combination thereof; and wherein the second polyethylene glycol-based compound is a MULTIARM-(5-50k)-SG, a MULTIARM-(5-50k)-SGA, or a combination thereof. In some embodiments, the MULTIARM of the first polyethylene glycol-based compound and/or the second polyethylene glycol-based compound is 3ARM, 4ARM, 6ARM, or 8ARM.

In some embodiments, the first polyethylene glycol-based compound is 4ARM-2k-NH2, 4ARM-5k-NH2, 8ARM-20k-NH2, 4ARM-20k-AA, 8ARM-20k-AA, or a combination thereof, and the second polyethylene glycol-based compound is 4ARM-10k-SG, 8ARM-15k-SG, 4ARM-20k-SGA, 4ARM-10k-SS, or a combination thereof.

In some embodiments, the first polyethylene glycol-based compound is 8ARM-20k-NH2 and/or 8ARM-20k-AA and the second polyethylene glycol-based compound is 4ARM-20k-SGA.

In some embodiments, the buffer provides a pH of about 6.0 to about 8.5.

In some embodiments, the composition that is free of a hemostasis agent is selected from the group consisting of aminocaproic acid, tranexamic acid, aminomethylbenzoic acid, aprotinin, alfal antitrypsin, Cl-inhibitor, camostat, Vitamin K, phytomenadione, menadione, fibrinogen, absorbable gelatin sponge, oxidized cellulose, tetragalacturonic acid hydroxymethylester, adrenalone, thrombin, collagen, calcium alginate, epinephrine, human fibrinogen, coagulation factor IX, II, VII and X in combination, coagulation factor VIII, factor VIII inhibitor bypassing activity, coagulation factor IX, coagulation factor VII, von Willebrand factor and coagulation factor VIII in combination, coagulation factor XIII, eptacog alfa, nonacog alfa, thrombin, etamsylate, carbazochrome, batroxobin, romiplostim, and eltrombopag.

In some embodiments, a methods of treating a wound of a mammal are provided. In some embodiments, the method comprises applying, administering, or placing the composition to a target site of the wound of the mammal, wherein the polyglycol-based, fully synthetic, biocompatible formulation gels at the target site of the wound of the mammal to form a polyglycol-based, fully synthetic, biocompatible hydrogel polymer.

In some embodiments, any of the compositions provided herein can comprise silver.

In some embodiments, a would healing solid polyglycol-based, fully synthetic, pre-formulation is provided, comprising: (a) at least one solid first compound comprising more than two nucleophilic groups; (b) at least one solid second compound comprising more than two electrophilic groups; (c) optionally, a solid buffer component; (d) optionally, a therapeutic agent; and (e) optionally, a solid viscosity enhancer, wherein the solid polyglycol-based, fully synthetic, pre-formulation polymerizes and/or gels at a target site of the wound to form a polyglycol-based, fully synthetic, biocompatible hydrogel polymer after addition of a liquid component, wherein the liquid component does not contain any first compound or second compound, and provided that the solid polyglycol-based, fully synthetic, pre-formulation does not contain any aqueous component.

In some embodiments, the wound healing solid polyglycol-based, fully synthetic, pre-formulation, wherein the therapeutic is a solid therapeutic agent.

In some embodiments, the liquid component comprises water, saline, a buffer, a therapeutic agent, or a combination thereof. In some embodiments, the solid first compound is a MULTIARM (5k-50k) polyol derivative comprising polyglycol subunits and more than two nucleophilic groups, and wherein the solid second compound is a MULTIARM (5k-50k) polyol derivative comprising polyglycol subunits and more than two electrophilic groups.

In some embodiments, the solid first compound is a MULTIARM-(5-50k)-SH, a MULTIARM-(5-50k)-NH2, a MULTIARM-(5-50k)-AA, or a combination thereof, and the solid second compound is a MULTIARM-(5-50k)-SG, a MULTIARM-(5-50k)-SGA, a MULTIARM-(5-50k)-SS, or a combination thereof.

In some embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof, and the solid second compound is 4ARM-10k-SG, 8ARM-15k-SG, 4ARM-20k-SGA, 4ARM-10k-SS, or a combination thereof.

In some embodiments, the solid first compound is 8ARM-20k-NH2 and/or 8ARM-20k-AA, and the solid second compound is 4ARM-20k-SGA.

In one aspect, provided herein is a solid polyglycol-based, fully synthetic, pre-formulation, comprising at least one solid first compound comprising more than two nucleophilic groups; and at least one solid second compound comprising more than two electrophilic groups; wherein the solid polyglycol-based, fully synthetic, pre-formulation polymerizes and/or gels to form a polyglycol-based, fully synthetic, biocompatible hydrogel polymer in after addition of a liquid component. In some embodiments, the solid polyglycol-based, fully synthetic, pre-formulation, further comprises a solid buffer component. In some embodiments, the liquid component comprises water, saline, a buffer, a therapeutic agent or a combination thereof. In certain embodiments, the liquid component comprises water. In certain embodiments, the liquid component comprises saline. In certain embodiments, the liquid component comprises a buffer. In certain embodiments, the liquid component comprises a therapeutic agent. In some embodiments, the polyglycol-based, fully synthetic, biocompatible hydrogel polymer at least partially adheres to a target site.

In certain embodiments, the solid polyglycol-based, fully synthetic, pre-formulation further comprises a viscosity enhancer. In some embodiments, the viscosity enhancer is selected from hydroxyethylcellulose, hydroxypropylmethylcellulose, methylcellulose, polyvinyl alcohol, or polyvinylpyrrolidone.

In some embodiments, the nucleophilic group comprises a thiol or amino group. In certain embodiments, the nucleophilic group comprises an amino group. In some embodiments, the solid first compound is a polyol derivative. In some embodiments, solid first compound is a trimethylolpropane, diglycerol, pentaerythritol, sorbitol, hexaglycerol, tripentaerythritol, or polyglycerol derivative. In certain embodiments, the solid first compound is a trimethylolpropane, pentaerythritol, hexaglycerol, or tripentaerythritol derivative. In some embodiments, the solid first compound is a pentaerythritol or hexaglycerol derivative. In certain embodiments, the solid first compound is selected from the group consisting of ethoxylated pentaerythritol ethylamine ether, ethoxylated pentaerythritol propylamine ether, ethoxylated pentaerythritol amino acetate, ethoxylated hexaglycerol ethylamine ether, ethoxylated hexaglycerol propylamine ether, and ethoxylated hexaglycerol amino acetate. In some embodiments, the solid first compound is a MULTIARM (5k-50k) polyol derivative comprising polyglycol subunits and more than two nucleophilic groups. In some embodiments, MULTIARM is 3ARM, 4ARM, 6ARM, 8ARM, 10ARM, 12ARM. In some embodiments, MULTIARM is 4ARM or 8ARM. In some embodiments, the solid first compound is a MULTIARM-(5-50k)-SH, a MULTIARM-(5-50k)-NH2, a MULTIARM-(5-50k)-AA, or a combination thereof. In certain embodiments, the solid first compound is 4ARM-(5k-50k)-SH, 4ARM-(5k-50k)-NH2, 4ARM-(5k-50k)-AA, 8ARM-(5k-50k)-NH2, 8ARM-(5k-50k)-AA, or a combination thereof. In some embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof.

In some embodiments, the solid first compound further comprises a solid second first compound comprising more than two nucleophilic groups. In some embodiments, the solid first compound further comprises a solid second first compound that is a MULTIARM-(5k-50k) polyol derivative comprising polyglycol subunits and more than two nucleophilic groups. In some embodiments, the solid second first compound is MULTIARM-(5-50k)-SH, MULTIARM-(5k-50k)-NH2, MULTIARM-(5k-50k)-AA. In some embodiments, the solid first compound is water soluble.

In certain embodiments, the electrophilic group is an epoxide, N-succinimidyl succinate, N-succinimidyl glutarate, N-succinimidyl succinamide or N-succinimidyl glutaramide. In some embodiments, the electrophilic group is N-succinimidyl glutaramide. In some embodiments, the solid second compound is a polyol derivative. In certain embodiments, the second compound is a trimethylolpropane, diglycerol, pentaerythritol, sorbitol, hexaglycerol, tripentaerythritol, or polyglycerol derivative. In some embodiments, the second compound is a trimethylolpropane, pentaerythritol, or hexaglycerol derivative. In certain embodiments, the solid second compound is selected from the group consisting of ethoxylated pentaerythritol succinimidyl succinate, ethoxylated pentaerythritol succinimidyl glutarate, ethoxylated pentaerythritol succinimidyl glutaramide, ethoxylated hexaglycerol succinimidyl succinate, ethoxylated hexaglycerol succinimidyl glutarate, and ethoxylated hexaglycerol succinimidyl glutaramide. In some embodiments, the solid second compound is a MULTIARM-(5k-50k) polyol derivative comprising polyglycol subunits and more than two electrophilic groups. In certain embodiments, the solid second compound is a MULTIARM-(5-50k)-SG, MULTIARM-(5-50k)-SGA, MULTIARM-(5-50k)-SS, MULTIARM-(5-50k)-SSA, or a combination thereof. In certain embodiments, the solid second compound is 4ARM-(5-50k)-SG, 4ARM-(5-50k)-SGA, 4ARM-(5-50k)-SS, 8ARM-(5-50k)-SG, 8ARM-(5-50k)-SGA, 8ARM-(5-50k)-SS, or a combination thereof. In some embodiments, the solid second compound is 4ARM-10k-SG, 8ARM-15k-SG, 4ARM-20k-SGA, 4ARM-10k-SS, or a combination thereof.

In some embodiments, the solid first compound is a MULTIARM-(5-50k)-SH, a MULTIARM-(5-50k)-NH2, a MULTIARM-(5-50k)-AA, or a combination thereof, and the solid second compound is a MULTIARM-(5-50k)-SG, a MULTIARM-(5-50k)-SGA, a MULTIARM-(5-50k)-SS, or a combination thereof. In other embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof, and the solid second compound is 4ARM-10k-SG, 8ARM-15k-SG, 4ARM-20k-SGA, 4ARM-10k-SS, or a combination thereof. In certain embodiments, the solid first compound is 8ARM-20k-NH2 and/or 8ARM-20k-AA, and the solid second compound is 4ARM-20k-SGA. In some embodiments, the solid second compound is water soluble.

In some embodiments, the solid polyglycol-based, fully synthetic, pre-formulation gels at a predetermined time to form the polyglycol-based, fully synthetic, biocompatible hydrogel polymer. In certain embodiments, the polyglycol-based, fully synthetic, biocompatible hydrogel polymer is bioabsorbable. In some embodiments, the polyglycol-based, fully synthetic, biocompatible hydrogel polymer is bioabsorbed within about 1 to 70 days. In certain embodiments, the polyglycol-based, fully synthetic, biocompatible hydrogel polymer is substantially non-bioabsorbable.

In some embodiments, the solid polyglycol-based, fully synthetic, pre-formulation further comprises a radiopaque material or a pharmaceutically acceptable dye. In certain embodiments, the radiopaque material is selected from sodium iodide, barium sulfate, tantalum, Visipaque®, Omnipaque®, or Hypaque®, or combinations thereof.

In some embodiments, the solid polyglycol-based, fully synthetic, pre-formulation further comprises one or more therapeutic agents. In certain embodiments, the therapeutic agent is an antibacterial agent, an antifungal agent, an immunosuppressant agent, an anti-inflammatory agent, a bisphosphonate, gallium nitrate, stem cells, an antiseptic agent, or a lubricity agent. In some embodiments, the anti-inflammatory agent is a corticosteroid or a TNF-α inhibitor. In some embodiments, the anti-inflammatory agent is a corticosteroid. In certain embodiments, the corticosteroid is trimacinolone or methylprednisolone. In some embodiments, the therapeutic agent is an antiseptic agent. In certain embodiments, the antiseptic agent is chlorhexidine. In some embodiments, the therapeutic agent is a lubricity agent. In certain embodiments, the lubricity agent is hyaluronic acid. In some embodiments, the therapeutic agent is released from the polyglycol-based, fully synthetic, biocompatible hydrogel polymer through diffusion, osmosis, degradation of the polyglycol-based, fully synthetic, biocompatible hydrogel polymer, or any combination thereof. In certain embodiments, the therapeutic agent is initially released from the polyglycol-based, fully synthetic, biocompatible hydrogel polymer through diffusion and later released through degradation of the polyglycol-based, fully synthetic, biocompatible hydrogel polymer. In some embodiments, the therapeutic agent is substantially released from the polyglycol-based, fully synthetic, biocompatible hydrogel polymer within 180 days. In certain embodiments, the therapeutic agent is substantially released from the polyglycol-based, fully synthetic, biocompatible hydrogel polymer within 14 days. In some embodiments, the therapeutic agent is substantially released from the polyglycol-based, fully synthetic, biocompatible hydrogel polymer within 24 hours. In certain embodiments, the therapeutic agent is substantially released from the polyglycol-based, fully synthetic, biocompatible hydrogel polymer within one hour. In some embodiments, the first compound and the second compound do not react with the therapeutic agent during formation of the polyglycol-based, fully synthetic, biocompatible hydrogel polymer. In certain embodiments, the polyglycol-based, fully synthetic, biocompatible hydrogel polymer interacts with the therapeutic agent, and wherein more than 10% of the therapeutic agent is released through degradation of the polyglycol-based, fully synthetic, biocompatible hydrogel polymer. In some embodiments, more than 30% of the therapeutic agent is released through degradation of the polyglycol-based, fully synthetic, biocompatible hydrogel polymer. In certain embodiments, the polyglycol-based, fully synthetic, biocompatible hydrogel polymer interacts with the therapeutic agent by forming covalent bonds between the polyglycol-based, fully synthetic, biocompatible hydrogel polymer and the therapeutic agent. In some embodiments, the polyglycol-based, fully synthetic, biocompatible hydrogel polymer interacts with the therapeutic agent by forming a non-covalent bond between the polyglycol-based, fully synthetic, biocompatible hydrogel polymer and the therapeutic agent. In some embodiments, the therapeutic agent is released while the polyglycol-based, fully synthetic, biocompatible hydrogel polymer degrades. In certain embodiments, the release of the therapeutic agent is essentially inhibited until a time that the polyglycol-based, fully synthetic, biocompatible hydrogel polymer starts to degrade. In some embodiments, the time the polyglycol-based, fully synthetic, biocompatible hydrogel polymer starts to degrade is longer the higher a degree of cross-linking of the polyglycol-based, fully synthetic, biocompatible hydrogel polymer. In certain embodiments, the time the polyglycol-based, fully synthetic, biocompatible hydrogel polymer starts to degrade is shorter the higher a concentration of ester groups in the first or second compound.

In one aspect, provided herein is a method of treating wounds of a mammal by delivering a liquid polyglycol-based, fully synthetic, biocompatible formulation formed by adding a liquid component to the solid polyglycol-based, fully synthetic, pre-formulation to a target site of the wound of the mammal, wherein the liquid polyglycol-based, fully synthetic, biocompatible formulation gels at the target site of the wound to form a polyglycol-based, fully synthetic, biocompatible hydrogel polymer. In another aspect, provided herein, is a method of treating arthritis in a mammal by delivering a liquid polyglycol-based, fully synthetic, biocompatible formulation formed by adding a liquid component to the solid polyglycol-based, fully synthetic, pre-formulation into a target site in a joint space, wherein the liquid polyglycol-based, fully synthetic, biocompatible formulation gels at the target site in the joint space to form a polyglycol-based, fully synthetic, biocompatible hydrogel polymer. In a further aspect, provided herein is a method of treating navicular disease in a horse by delivering a liquid polyglycol-based, fully synthetic, biocompatible formulation formed by adding a liquid component to the solid polyglycol-based, fully synthetic, pre-formulation to a target site in a hoof of the horse, wherein the liquid polyglycol-based, fully synthetic, biocompatible formulation gels at the target site in the hoof of the horse to form a polyglycol-based, fully synthetic, biocompatible hydrogel polymer. In certain embodiments of methods described herein, the polyglycol-based, fully synthetic, biocompatible hydrogel polymer closes the wound. In some embodiments, the polyglycol-based, fully synthetic, biocompatible hydrogel polymer covers the wound and adheres to surrounding skin. In some embodiments, the mammal is a human. In certain embodiments, the mammal is an animal. In some embodiments, the animal is a dog, cat, cow, pig, or horse.

In some embodiments, the polyglycol-based, fully synthetic, biocompatible hydrogel polymer of the synthetic, pre-formulation as described herein.

In another aspect, provided herein is a polyglycol-based, fully synthetic, biocompatible polymer, is formed by contacting a solid polyglycol-based, fully synthetic, pre-formulation with a liquid component, comprising at least one solid first compound comprising more than two nucleophilic groups; and at least one solid second compound comprising more than two electrophilic groups. In some embodiments, the solid polyglycol-based, fully synthetic, pre-formulation further comprises a solid buffer component. In some embodiments, the polyglycol-based, fully synthetic, pre-formulation further comprises a therapeutic agent. In certain embodiments, the liquid component comprises water, saline, a buffer, a therapeutic agent or a combination thereof. In some embodiments, the liquid component comprises water. In other embodiments, the liquid component comprises saline. In some embodiments, the liquid component comprises a buffer. In certain embodiments, the liquid component comprises a therapeutic agent.

In some embodiments, the liquid component comprises of water. In some embodiments, the polyglycol-based, fully synthetic solid pre-formulation further comprises a viscosity enhancer. In some embodiments, the polyglycol-based fully synthetic, pre-formulation further comprises a therapeutic agent.

In another aspect, described herein is a solid pre-formulation, comprising at least one solid first compound comprising more than two nucleophilic groups; and at least one solid second compound comprising more than two electrophilic groups; wherein the pre-formulation polymerizes and/or gels form a biocompatible hydrogel polymer in the presence of a liquid component. In some embodiments, the solid pre-formulation further comprises a solid buffer component. In certain embodiments, the liquid component comprises water, saline, a buffer, a therapeutic agent or a combination thereof. In some embodiments, the liquid component comprises water. In certain embodiments, the liquid component comprises saline. In some embodiments, the liquid component comprises a buffer. In some embodiments, the liquid component comprises a therapeutic agent. In certain embodiments, the hydrogel polymer at least partially adheres to a target site. In some embodiments, the solid pre-formulation further comprises a viscosity enhancer. In certain embodiments, the viscosity enhancer is selected from hydroxyethylcellulose, hydroxypropylmethylcellulose, methylcellulose, polyvinyl alcohol, or polyvinylpyrrolidone

In certain embodiments, the solid pre-formulation further comprises a therapeutic agent. In some embodiments, the therapeutic agent is an antibacterial agent, an antifungal agent, an immunosuppressant agent, an anti-inflammatory agent, a bisphosphonate, gallium nitrate, stem cells, an antiseptic agent, or a lubricity agent. In certain embodiments, anti-inflammatory is s a corticosteroid or a TNF-α inhibitor. In some embodiments, the therapeutic agent is an antiseptic agent.

In some embodiments, the solid pre-formulation is polyglycol-based. In other embodiments, the solid pre-formulation is fully synthetic. In certain embodiments, the solid pre-formulation is PEG-based. In some embodiments, the solid pre-formulation is fully synthetic and polyglycol based. In other embodiments, the solid pre-formulation is fully synthetic and PEG-based.

In another aspect described herein is a solid biocompatible hydrogel polymer, comprising at least one solid first monomeric unit bound through at least one amide, thioester, or thioether linkage to at least one solid second monomeric unit; and at least one solid second monomeric unit bound to at least one solid first monomeric unit; wherein biocompatible hydrogel polymer is formed from contacting a solid pre-formulation with a liquid component. In some embodiments, the liquid component comprises water, saline solution, therapeutic agent, or a combination thereof. In certain embodiments, the liquid component comprises water. In some embodiments, the liquid component comprises a saline solution. In certain embodiments, the liquid component comprises a therapeutic agent. In some embodiments, the solid first monomeric unit is a polyol derivative. In certain embodiments, the solid first monomeric unit is a glycol, trimethylolpropane, pentaerythritol, hexaglycerol, or tripentaerythritol derivative. In some embodiments, the solid first monomeric unit further comprises one or more polyethylene glycol sections. In certain embodiments, the solid first monomeric unit is a pentaerythritol or hexaglycerol derivative. In some embodiments, the solid second monomeric unit is a polyol derivative. In certain embodiments, the solid second monomeric unit is a trimethylolpropane, glycerol, diglycerol, pentaerythritol, sorbitol, hexaglycerol, tripentaerythritol, or polyglycerol derivative. In some embodiments, the solid second monomeric further comprises one or more polyethylene glycol sections. In certain embodiments, the solid second monomeric unit is a trimethylolpropane, pentaerythritol, or hexaglycerol derivative.

In another aspect described herein is a biocompatible hydrogel polymer, comprising: at least one solid first monomeric unit bound through at least one amide linkage to at least one solid second monomeric unit; and at least one solid second monomeric unit bound to at least one solid first monomeric unit; wherein the biocompatible hydrogel polymer is formed from contacting a solid pre-formulation with a liquid component. In some embodiments, the liquid component comprises water, saline solution, saline solution, therapeutic agent, or combination thereof. In certain embodiments, the liquid component comprises water. In some embodiments, the liquid component comprises a saline solution. In certain embodiments, the liquid component comprises a therapeutic agent. In some embodiments, the solid first monomeric unit is a polyol derivative. In certain embodiments, the solid first monomeric unit is a glycol, trimethylolpropane, pentaerythritol, hexaglycerol, or tripentaerythritol derivative. In some embodiments, the solid first monomeric unit further comprises one or more polyethylene glycol sections. In certain embodiments, the solid first monomeric unit is a pentaerythritol or hexaglycerol derivative. In some embodiments, the solid second monomeric unit is a polyol derivative. In certain embodiments, the solid second monomeric unit is a trimethylolpropane, glycerol, diglycerol, pentaerythritol, sorbitol, hexaglycerol, tripentaerythritol, or polyglycerol derivative. In some embodiments, the solid second monomeric further comprises one or more polyethylene glycol sections. In certain embodiments, the solid second monomeric unit is a trimethylolpropane, pentaerythritol, or hexaglycerol derivative.

In some embodiments, a solid polyglycol-based, fully synthetic, pre-formulation, comprising at least one solid first compound comprising more than two nucleophilic groups; and at least one solid second compound comprising more than two electrophilic groups; wherein the solid polyglycol-based, fully synthetic, pre-formulation polymerizes and/or gels to form a polyglycol-based, fully synthetic, biocompatible hydrogel polymer in after addition of a liquid component. In some embodiments, the solid polyglycol-based, fully synthetic, pre-formulation, further comprises a solid buffer component. In some embodiments, the liquid component comprises water, saline, a buffer, a therapeutic agent or a combination thereof. In certain embodiments, the liquid component comprises water. In certain embodiments, the liquid component comprises saline. In certain embodiments, the liquid component comprises a buffer. In certain embodiments, the liquid component comprises a therapeutic agent. In some embodiments, the polyglycol-based, fully synthetic, biocompatible hydrogel polymer at least partially adheres to a target site.

In certain embodiments, the solid polyglycol-based, fully synthetic, pre-formulation further comprises a viscosity enhancer. In some embodiments, the viscosity enhancer is selected from hydroxyethylcellulose, hydroxypropylmethylcellulose, methylcellulose, polyvinyl alcohol, or polyvinylpyrrolidone.

In some embodiments, the nucleophilic group comprises a thiol or amino group. In certain embodiments, the nucleophilic group comprises an amino group. In some embodiments, the solid first compound is a polyol derivative. In some embodiments, solid first compound is a trimethylolpropane, diglycerol, pentaerythritol, sorbitol, hexaglycerol, tripentaerythritol, or polyglycerol derivative. In certain embodiments, the solid first compound is a trimethylolpropane, pentaerythritol, hexaglycerol, or tripentaerythritol derivative. In some embodiments, the solid first compound is a pentaerythritol or hexaglycerol derivative. In certain embodiments, the solid first compound is selected from the group consisting of ethoxylated pentaerythritol ethylamine ether, ethoxylated pentaerythritol propylamine ether, ethoxylated pentaerythritol amino acetate, ethoxylated hexaglycerol ethylamine ether, ethoxylated hexaglycerol propylamine ether, and ethoxylated hexaglycerol amino acetate. In some embodiments, the solid first compound is a MULTIARM (5k-50k) polyol derivative comprising polyglycol subunits and more than two nucleophilic groups. In some embodiments, MULTIARM is 3ARM, 4ARM, 6ARM, 8ARM, 10ARM, 12ARM. In some embodiments, MULTIARM is 4ARM or 8ARM. In some embodiments, the solid first compound is a MULTIARM-(5-50k)-SH, a MULTIARM-(5-50k)-NH2, a MULTIARM-(5-50k)-AA, or a combination thereof. In certain embodiments, the solid first compound is 4ARM-(5k-50k)-SH, 4ARM-(5k-50k)-NH2, 4ARM-(5k-50k)-AA, 8ARM-(5k-50k)-NH2, 8ARM-(5k-50k)-AA, or a combination thereof. In some embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof.

In some embodiments, the solid first compound further comprises a solid second first compound comprising more than two nucleophilic groups. In some embodiments, the solid first compound further comprises a solid second first compound that is a MULTIARM-(5k-50k) polyol derivative comprising polyglycol subunits and more than two nucleophilic groups. In some embodiments, the solid second first compound is MULTIARM-(5-50k)-SH, MULTIARM-(5k-50k)-NH2, MULTIARM-(5k-50k)-AA. In some embodiments, the solid first compound is water soluble.

In certain embodiments, the electrophilic group is an epoxide, N-succinimidyl succinate, N-succinimidyl glutarate, N-succinimidyl succinamide or N-succinimidyl glutaramide. In some embodiments, the electrophilic group is N-succinimidyl glutaramide. In some embodiments, the solid second compound is a polyol derivative. In certain embodiments, the second compound is a trimethylolpropane, diglycerol, pentaerythritol, sorbitol, hexaglycerol, tripentaerythritol, or polyglycerol derivative. In some embodiments, the second compound is a trimethylolpropane, pentaerythritol, or hexaglycerol derivative. In certain embodiments, the solid second compound is selected from the group consisting of ethoxylated pentaerythritol succinimidyl succinate, ethoxylated pentaerythritol succinimidyl glutarate, ethoxylated pentaerythritol succinimidyl glutaramide, ethoxylated hexaglycerol succinimidyl succinate, ethoxylated hexaglycerol succinimidyl glutarate, and ethoxylated hexaglycerol succinimidyl glutaramide. In some embodiments, the solid second compound is a MULTIARM-(5k-50k) polyol derivative comprising polyglycol subunits and more than two electrophilic groups. In certain embodiments, the solid second compound is a MULTIARM-(5-50k)-SG, MULTIARM-(5-50k)-SGA, MULTIARM-(5-50k)-SS, MULTIARM-(5-50k)-SSA, or a combination thereof. In certain embodiments, the solid second compound is 4ARM-(5-50k)-SG, 4ARM-(5-50k)-SGA, 4ARM-(5-50k)-SS, 8ARM-(5-50k)-SG, 8ARM-(5-50k)-SGA, 8ARM-(5-50k)-SS, or a combination thereof. In some embodiments, the solid second compound is 4ARM-10k-SG, 8ARM-15k-SG, 4ARM-20k-SGA, 4ARM-10k-SS, or a combination thereof.

In some embodiments, the solid first compound is a MULTIARM-(5-50k)-SH, a MULTIARM-(5-50k)-NH2, a MULTIARM-(5-50k)-AA, or a combination thereof, and the solid second compound is a MULTIARM-(5-50k)-SG, a MULTIARM-(5-50k)-SGA, a MULTIARM-(5-50k)-SS, or a combination thereof. In other embodiments, the solid first compound is 4ARM-5k-SH, 4ARM-2k-NH2, 4ARM-5k-NH2, 8ARM-20k-NH2, 4ARM-20k-AA, 8ARM-20k-AA, or a combination thereof, and the solid second compound is 4ARM-10k-SG, 8ARM-15k-SG, 4ARM-20k-SGA, 4ARM-10k-SS, or a combination thereof. In certain embodiments, the solid first compound is 8ARM-20k-NH2 and/or 8ARM-20k-AA, and the solid second compound is 4ARM-20k-SGA. In some embodiments, the solid second compound is water soluble.

In some embodiments, the solid polyglycol-based, fully synthetic, pre-formulation gels at a predetermined time to form the polyglycol-based, fully synthetic, biocompatible hydrogel polymer. In certain embodiments, the polyglycol-based, fully synthetic, biocompatible hydrogel polymer is bioabsorbable. In some embodiments, the polyglycol-based, fully synthetic, biocompatible hydrogel polymer is bioabsorbed within about 1 to 70 days. In certain embodiments, the polyglycol-based, fully synthetic, biocompatible hydrogel polymer is substantially non-bioabsorbable.

In some embodiments, the solid polyglycol-based, fully synthetic, pre-formulation further comprises a radiopaque material or a pharmaceutically acceptable dye. In certain embodiments, the radiopaque material is selected from sodium iodide, barium sulfate, tantalum, Visipaque®, Omnipaque®, or Hypaque®, or combinations thereof.

In some embodiments, the solid polyglycol-based, fully synthetic, pre-formulation further comprises one or more therapeutic agents. In certain embodiments, the therapeutic agent is an antibacterial agent, an antifungal agent, an immunosuppressant agent, an anti-inflammatory agent, a bisphosphonate, gallium nitrate, stem cells, an antiseptic agent, or a lubricity agent. In some embodiments, the anti-inflammatory agent is a corticosteroid or a TNF-α inhibitor. In some embodiments, the anti-inflammatory agent is a corticosteroid. In certain embodiments, the corticosteroid is trimacinolone or methylprednisolone. In some embodiments, the therapeutic agent is an antiseptic agent. In certain embodiments, the antiseptic agent is chlorhexidine. In some embodiments, the therapeutic agent is a lubricity agent. In certain embodiments, the lubricity agent is hyaluronic acid. In some embodiments, the therapeutic agent is released from the polyglycol-based, fully synthetic, biocompatible hydrogel polymer through diffusion, osmosis, degradation of the polyglycol-based, fully synthetic, biocompatible hydrogel polymer, or any combination thereof. In certain embodiments, the therapeutic agent is initially released from the polyglycol-based, fully synthetic, biocompatible hydrogel polymer through diffusion and later released through degradation of the polyglycol-based, fully synthetic, biocompatible hydrogel polymer. In some embodiments, the therapeutic agent is substantially released from the polyglycol-based, fully synthetic, biocompatible hydrogel polymer within 180 days. In certain embodiments, the therapeutic agent is substantially released from the polyglycol-based, fully synthetic, biocompatible hydrogel polymer within 14 days. In some embodiments, the therapeutic agent is substantially released from the polyglycol-based, fully synthetic, biocompatible hydrogel polymer within 24 hours. In certain embodiments, the therapeutic agent is substantially released from the polyglycol-based, fully synthetic, biocompatible hydrogel polymer within one hour. In some embodiments, the first compound and the second compound do not react with the therapeutic agent during formation of the polyglycol-based, fully synthetic, biocompatible hydrogel polymer. In certain embodiments, the polyglycol-based, fully synthetic, biocompatible hydrogel polymer interacts with the therapeutic agent, and wherein more than 10% of the therapeutic agent is released through degradation of the polyglycol-based, fully synthetic, biocompatible hydrogel polymer. In some embodiments, more than 30% of the therapeutic agent is released through degradation of the polyglycol-based, fully synthetic, biocompatible hydrogel polymer. In certain embodiments, the polyglycol-based, fully synthetic, biocompatible hydrogel polymer interacts with the therapeutic agent by forming covalent bonds between the polyglycol-based, fully synthetic, biocompatible hydrogel polymer and the therapeutic agent. In some embodiments, the polyglycol-based, fully synthetic, biocompatible hydrogel polymer interacts with the therapeutic agent by forming a non-covalent bond between the polyglycol-based, fully synthetic, biocompatible hydrogel polymer and the therapeutic agent. In some embodiments, the therapeutic agent is released while the polyglycol-based, fully synthetic, biocompatible hydrogel polymer degrades. In certain embodiments, the release of the therapeutic agent is essentially inhibited until a time that the polyglycol-based, fully synthetic, biocompatible hydrogel polymer starts to degrade. In some embodiments, the time the polyglycol-based, fully synthetic, biocompatible hydrogel polymer starts to degrade is longer the higher a degree of cross-linking of the polyglycol-based, fully synthetic, biocompatible hydrogel polymer. In certain embodiments, the time the polyglycol-based, fully synthetic, biocompatible hydrogel polymer starts to degrade is shorter the higher a concentration of ester groups in the first or second compound.

In one aspect, provided herein is a method of treating wounds of a mammal by delivering a liquid polyglycol-based, fully synthetic, biocompatible formulation formed by adding a liquid component to the solid polyglycol-based, fully synthetic, pre-formulation to a target site of the wound of the mammal, wherein the liquid polyglycol-based, fully synthetic, biocompatible formulation gels at the target site of the wound to form a polyglycol-based, fully synthetic, biocompatible hydrogel polymer. In another aspect, provided herein, is a method of treating arthritis in a mammal by delivering a liquid polyglycol-based, fully synthetic, biocompatible formulation formed by adding a liquid component to the solid polyglycol-based, fully synthetic, pre-formulation into a target site in a joint space, wherein the liquid polyglycol-based, fully synthetic, biocompatible formulation gels at the target site in the joint space to form a polyglycol-based, fully synthetic, biocompatible hydrogel polymer. In a further aspect, provided herein is a method of treating navicular disease in a horse by delivering a liquid polyglycol-based, fully synthetic, biocompatible formulation formed by adding a liquid component to the solid polyglycol-based, fully synthetic, pre-formulation to a target site in a hoof of the horse, wherein the liquid polyglycol-based, fully synthetic, biocompatible formulation gels at the target site in the hoof of the horse to form a polyglycol-based, fully synthetic, biocompatible hydrogel polymer. In certain embodiments of methods described herein, the polyglycol-based, fully synthetic, biocompatible hydrogel polymer closes the wound. In some embodiments, the polyglycol-based, fully synthetic, biocompatible hydrogel polymer covers the wound and adheres to surrounding skin. In some embodiments, the mammal is a human. In certain embodiments, the mammal is an animal. In some embodiments, the animal is a dog, cat, cow, pig, or horse.

In some embodiments, the polyglycol-based, fully synthetic, biocompatible hydrogel polymer of the synthetic, pre-formulation as described herein.

In another aspect, provided herein is a polyglycol-based, fully synthetic, biocompatible polymer, is formed by contacting a solid polyglycol-based, fully synthetic, pre-formulation with a liquid component, comprising at least one solid first compound comprising more than two nucleophilic groups; and at least one solid second compound comprising more than two electrophilic groups. In some embodiments, the solid polyglycol-based, fully synthetic, pre-formulation further comprises a solid buffer component. In some embodiments, the polyglycol-based, fully synthetic, pre-formulation further comprises a therapeutic agent. In certain embodiments, the liquid component comprises water, saline, a buffer, a therapeutic agent or a combination thereof. In some embodiments, the liquid component comprises water. In other embodiments, the liquid component comprises saline. In some embodiments, the liquid component comprises a buffer. In certain embodiments, the liquid component comprises a therapeutic agent. In some embodiments, the liquid component comprises of water. In some embodiments, the polyglycol-based, fully synthetic solid pre-formulation further comprises a viscosity enhancer. In some embodiments, the polyglycol-based fully synthetic, pre-formulation further comprises a therapeutic agent.

In another aspect, described herein is a solid pre-formulation, comprising at least one solid first compound comprising more than two nucleophilic groups; and at least one solid second compound comprising more than two electrophilic groups; wherein the pre-formulation polymerizes and/or gels form a biocompatible hydrogel polymer in the presence of a liquid component. In some embodiments, the solid pre-formulation further comprises a solid buffer component. In certain embodiments, the liquid component comprises water, saline, a buffer, a therapeutic agent or a combination thereof. In some embodiments, the liquid component comprises water. In certain embodiments, the liquid component comprises saline. In some embodiments, the liquid component comprises a buffer. In some embodiments, the liquid component comprises a therapeutic agent. In certain embodiments, the hydrogel polymer at least partially adheres to a target site. In some embodiments, the solid pre-formulation further comprises a viscosity enhancer. In certain embodiments, the viscosity enhancer is selected from hydroxyethylcellulose, hydroxypropylmethylcellulose, methylcellulose, polyvinyl alcohol, or polyvinylpyrrolidone

In certain embodiments, the solid pre-formulation further comprises a therapeutic agent. In some embodiments, the therapeutic agent is an antibacterial agent, an antifungal agent, an immunosuppressant agent, an anti-inflammatory agent, a bisphosphonate, gallium nitrate, stem cells, an antiseptic agent, or a lubricity agent. In certain embodiments, anti-inflammatory is s a corticosteroid or a TNF-α inhibitor. In some embodiments, the therapeutic agent is an antiseptic agent.

In some embodiments, the solid pre-formulation is polyglycol-based. In other embodiments, the solid pre-formulation is fully synthetic. In certain embodiments, the solid pre-formulation is PEG-based. In some embodiments, the solid pre-formulation is fully synthetic and polyglycol based. In other embodiments, the solid pre-formulation is fully synthetic and PEG-based.

In another aspect described herein is a solid biocompatible hydrogel polymer, comprising at least one solid first monomeric unit bound through at least one amide, thioester, or thioether linkage to at least one solid second monomeric unit; and at least one solid second monomeric unit bound to at least one solid first monomeric unit; wherein biocompatible hydrogel polymer is formed from contacting a solid pre-formulation with a liquid component. In some embodiments, the liquid component comprises water, saline solution, therapeutic agent, or a combination thereof. In certain embodiments, the liquid component comprises water. In some embodiments, the liquid component comprises a saline solution. In certain embodiments, the liquid component comprises a therapeutic agent. In some embodiments, the solid first monomeric unit is a polyol derivative. In certain embodiments, the solid first monomeric unit is a glycol, trimethylolpropane, pentaerythritol, hexaglycerol, or tripentaerythritol derivative. In some embodiments, the solid first monomeric unit further comprises one or more polyethylene glycol sections. In certain embodiments, the solid first monomeric unit is a pentaerythritol or hexaglycerol derivative. In some embodiments, the solid second monomeric unit is a polyol derivative. In certain embodiments, the solid second monomeric unit is a trimethylolpropane, glycerol, diglycerol, pentaerythritol, sorbitol, hexaglycerol, tripentaerythritol, or polyglycerol derivative. In some embodiments, the solid second monomeric further comprises one or more polyethylene glycol sections. In certain embodiments, the solid second monomeric unit is a trimethylolpropane, pentaerythritol, or hexaglycerol derivative.

In another aspect described herein is a biocompatible hydrogel polymer, comprising: at least one solid first monomeric unit bound through at least one amide linkage to at least one solid second monomeric unit; and at least one solid second monomeric unit bound to at least one solid first monomeric unit; wherein the biocompatible hydrogel polymer is formed from contacting a solid pre-formulation with a liquid component. In some embodiments, the liquid component comprises water, saline solution, saline solution, therapeutic agent, or combination thereof. In certain embodiments, the liquid component comprises water. In some embodiments, the liquid component comprises a saline solution. In certain embodiments, the liquid component comprises a therapeutic agent. In some embodiments, the solid first monomeric unit is a polyol derivative. In certain embodiments, the solid first monomeric unit is a glycol, trimethylolpropane, pentaerythritol, hexaglycerol, or tripentaerythritol derivative. In some embodiments, the solid first monomeric unit further comprises one or more polyethylene glycol sections. In certain embodiments, the solid first monomeric unit is a pentaerythritol or hexaglycerol derivative. In some embodiments, the solid second monomeric unit is a polyol derivative. In certain embodiments, the solid second monomeric unit is a trimethylolpropane, glycerol, diglycerol, pentaerythritol, sorbitol, hexaglycerol, tripentaerythritol, or polyglycerol derivative. In some embodiments, the solid second monomeric further comprises one or more polyethylene glycol sections. In certain embodiments, the solid second monomeric unit is a trimethylolpropane, pentaerythritol, or hexaglycerol derivative.

As used herein, the term “subject” refers to an animal such as a human, cat, dog, horse, pig, mouse, rat, or other mammal.

In some embodiments, the compositions provided for herein and throughout are free of biological materials. In some embodiments, the compositions provided for herein and throughout is free of any active ingredient. In some embodiments, the only active ingredient present in the composition is silver. An active ingredient is an agent that actively treats the wound, such as an antimicrobial, antibiotic, antiviral, antifungal, and the like. A non-limiting example of an active ingredient is silver, which can act as an antimicrobial. As used herein, the term “active ingredient” does not include a hydrogel bandage or other similar bandages. In some embodiments, the active ingredient is an anti-inflammatory, such as, but not limited to, a steroid or a NSAID.

In some embodiments, the compositions provided for herein and throughout are free of hemostasis agents, such as, but not limited to, those described herein.

### Area of for Treatment—Target Sites

In certain embodiments, the target site is inside a mammal. In some embodiments, the target site is inside a human being. In certain embodiments, the target site is on the human body. In some embodiments, the target site is accessible through surgery. In certain embodiments, the target site is accessible through minimally invasive surgery. In some embodiments, the target site is accessible through an endoscopic device. In certain embodiments, the target site is a wound on the skin of a mammal. In other embodiments, the target site is in a joint or on a bone of a mammal. In some embodiments, the target site is a surgical site in a mammal

In some embodiments, a biocompatible pre-formulation or a biocompatible hydrogel polymer matrix is used as a sealant, bandage, or adhesive. In certain embodiments, the biocompatible pre-formulation or biocompatible hydrogel polymer matrix is used to seal or bandage a wound on a mammal. In other embodiments, the biocompatible pre-formulation or biocompatible hydrogel polymer matrix is used to fill cavities, e.g., in a joint space to form a gel cushion. In other embodiments, the biocompatible pre-formulation or biocompatible hydrogel polymer matrix is used as a carrier for delivery of cells to target sites.

In some embodiments, the biocompatible hydrogel polymer matrix formulation is polymerized ex vivo. In certain embodiments, the ex vivo polymerized biocompatible hydrogel polymer matrix formulation is delivered through traditional routes of administration (e.g., oral, implantation, or rectal). In other embodiments, the ex vivo polymerized biocompatible hydrogel polymer matrix formulation is delivered during surgery to a target site.

### Delivery of the Biocompatible Hydrogel Formulation to a Target Site

In some embodiments, the biocompatible pre-formulation is delivered as a biocompatible pre-formulation to a target site through a catheter or a needle to form a biocompatible hydrogel polymer matrix at the target site. In other embodiments, the biocompatible pre-formulation is delivered to the target site in or on the mammal using syringe and needle. In some embodiments, a delivery device is used to deliver the biocompatible pre-formulation to the target site. In some embodiments, the biocompatible pre-formulation is delivered to the target site so that the biocompatible pre-formulation mostly covers the target site. In certain embodiments, the biocompatible pre-formulation substantially covers an exposed portion of diseased tissue. In some embodiments, the biocompatible pre-formulation does not spread to any other location intentionally. In some embodiments, the biocompatible pre-formulation substantially covers diseased tissue and does not significantly cover healthy tissue. In certain embodiments, the biocompatible hydrogel polymer matrix does not significantly cover healthy tissue. In some embodiments, the biocompatible pre-formulation gels over the target site and thoroughly covers diseased tissue. In some embodiments, the biocompatible hydrogel polymer matrix adheres to tissue. In some embodiments, the biocompatible hydrogel polymer matrix mixture gels after delivery at the target site, covering the target site. In some embodiments, the biocompatible hydrogel polymer matrix mixture gels prior to delivery at the target site.

In some embodiments, the gelling time of the biocompatible pre-formulation is set according to the preference of the doctor delivering the biocompatible pre-formulation mixture to a target site. In some embodiments, a physician delivers the biocompatible pre-formulation mixture to the target within 15 to 30 seconds. In certain embodiments, the gelling time is between about 20 seconds and 10 minutes. In some embodiments, the gelling time or curing time of the biocompatible pre-formulation is controlled by the pH of the aqueous buffer. In certain embodiments, the gelling time or curing time of the biocompatible pre-formulation is controlled by the selection of the first and second compounds. In some embodiments, the concentration of nucleophilic or electrophilic groups in the first or second compound influences the gelling time of the biocompatible pre-formulation. In some embodiments, cell concentration influences the gelling time of the biocompatible pre-formulation. In some embodiments, cell type influences the gelling time of the biocompatible pre-formulation. In some embodiments, optional addition components influence the gelling time of the biocompatible pre-formulation.

In some embodiments, curing of the biocompatible hydrogel polymer matrix is verified post-administration. In certain embodiments, the verification is performed in vivo at the delivery site. In other embodiments, the verification is performed ex vivo. In some embodiments, curing of the biocompatible hydrogel polymer matrix is verified visually through the fiber-optics of an endoscopic device. In certain embodiments, curing of biocompatible hydrogel polymer matrices comprising radiopaque materials is verified using X-ray, fluoroscopy, or computed tomography (CT) imaging. A lack of flow of the biocompatible hydrogel polymer matrix indicates that the biocompatible hydrogel polymer matrix has gelled and the biocompatible hydrogel is sufficiently cured. In further embodiments, curing of the biocompatible hydrogel polymer matrix is verified by evaluation of the residue in the delivery device, for instance the residue in the catheter of the bronchoscope or other endoscopic device, or the residue in the syringe used to deliver the biocompatible hydrogel polymer matrix. In other embodiments, curing of the biocompatible hydrogel polymer matrix is verified by depositing a small sample (e.g., ˜1 mL) on a piece of paper or in a small vessel and subsequent evaluation of the flow characteristics after the gelling time has passed.

Bioabsorbance of the Biocompatible Hydrogel Polymer matrix

In some embodiments, the biocompatible hydrogel polymer matrix is a bioabsorbable polymer. In certain embodiments, the biocompatible hydrogel polymer matrix is bioabsorbed within about 5 to 30 days. In some embodiments, the biocompatible hydrogel polymer matrix is bioabsorbed within about 30 to 180 days. In some embodiments, the biocompatible hydrogel polymer matrix is bioabsorbed within about 1 to 70 days. In some embodiments, the biocompatible hydrogel polymer matrix is bioabsorbed within about 14 to 180 days. In some embodiments the biocompatible hydrogel polymer matrix is bioabsorbed within about 365 days, 180 days, about 150 days, about 120 days, about 90 days, about 80 days, about 70 days, about 60 days, about 50 days, about 40 days, about 35 days, about 30 days, about 28 days, about 21 days, about 14 days, about 10 days, about 7 days, about 6 days, about 5 days, about 4 days, about 3 days, about 2 days, or about 1 day. In certain embodiments the biocompatible hydrogel polymer matrix is bioabsorbed within less than 365 days, 180 days, less than 150 days, less than 120 days, less than 90 days, less than 80 days, less than 70 days, less than 60 days, less than 50 days, less than 40 days, less than 35 days, less than 30 days, less than 28 days, less than 21 days, less than 14 days, less than 10 days, less than 7 days, less than 6 days, less than 5 days, less than 4 days, less than 3 days, less than 2 days, or less than 1 day. In some embodiments the biocompatible hydrogel polymer matrix is bioabsorbed within more than 365 days, 180 days, more than 150 days, more than 120 days, more than 90 days, more than 80 days, more than 70 days, more than 60 days, more than 50 days, more than 40 days, more than 35 days, more than 30 days, more than 28 days, more than 21 days, more than 14 days, more than 10 days, more than 7 days, more than 6 days, more than 5 days, more than 4 days, more than 3 days, more than 2 days, or more than 1 day. In some embodiments, the biocompatible hydrogel polymer matrix is substantially non-bioabsorbable.

The biocompatible hydrogel polymer matrix can be slowly bioabsorbed, dissolved, and or excreted. In some instances, the rate of bioabsorption is controlled by the number of ester groups in the biocompatible and/or biodegradable hydrogel polymer matrix. In other instances, the higher the concentration of ester units is in the biocompatible hydrogel polymer matrix, the longer is its lifetime in the body. In further instances, the electron density at the carbonyl of the ester unit controls the lifetime of the biocompatible hydrogel polymer matrix in the body. In certain instances, biocompatible hydrogel polymer matrices without ester groups are essentially not biodegradable. In additional instances, the molecular weight of the first and second compounds controls the lifetime of the biocompatible hydrogel polymer matrix in the body. In further instances, the number of ester groups per gram of polymer matrix controls the lifetime of the biocompatible hydrogel polymer matrix in the body.

In some instances, the lifetime of the biocompatible hydrogel polymer matrix can be estimated using a model, which controls the temperature and pH at physiological levels while exposing the biocompatible hydrogel polymer matrix to a buffer solution. In certain instances, the biodegradation of the biocompatible hydrogel polymer matrix is substantially non-enzymatic degradation.

In some embodiments, the selection of reaction conditions determines the degradation time of the biocompatible hydrogel polymer matrix. In certain embodiments, the concentration of the first compound and second compound monomers determines the degradation time of the resulting biocompatible hydrogel polymer matrix. In some instances, a higher monomer concentration leads to a higher degree of cross-linking in the resulting biocompatible hydrogel polymer matrix. In certain instances, more cross-linking leads to a later degradation of the biocompatible hydrogel polymer matrix. In certain embodiments, temperature determines the degradation time of the resulting biocompatible hydrogel polymer matrix. In some instances, a higher monomer concentration leads to a higher degree of cross-linking in the resulting biocompatible hydrogel polymer matrix.

In certain embodiments, the composition of the linker in the first and/or second compound influences the speed of degradation of the resulting biocompatible hydrogel polymer matrix. In some embodiments, the more ester groups are present in the biocompatible hydrogel polymer matrix, the faster the degradation of the biocompatible hydrogel polymer matrix. In certain embodiments, the higher the concentration of mercaptopropionate (ETTMP), acetate amine (AA), glutarate or succinate (SG or SS) monomers, the faster the rate of degradation.

In certain embodiments, the composition of the cell influences the speed of degradation of the resulting biocompatible hydrogel polymer matrix. In certain embodiments, the concentration of the cell influences the speed of degradation of the resulting biocompatible hydrogel polymer matrix. In certain embodiments, the composition of a buffer influences the speed of degradation of the resulting biocompatible hydrogel polymer matrix. In certain embodiments, the concentration of a buffer influences the speed of degradation of the resulting biocompatible hydrogel polymer matrix. In certain embodiments, the pH of a buffer influences the speed of degradation of the resulting biocompatible hydrogel polymer matrix. In certain embodiments, the composition of the optional additional components influences the speed of degradation of the resulting biocompatible hydrogel polymer matrix.

### Pre-Formulations and Hydrogel Matrices for Cell Delivery in the Treatment of Disease

In some embodiments, the biocompatible pre-formulation or hydrogel polymer matrix described herein is delivered to a target site on or in a mammal. In certain embodiments, the biocompatible pre-formulation or hydrogel polymer matrix is delivered to a target site in a joint. In some embodiments, the biocompatible pre-formulation forms a biocompatible hydrogel polymer matrix inside a joint. In certain embodiments, the biocompatible pre-formulation forms a sticky biocompatible polymer matrix to seal a wound on or in an animal. In some embodiments, the biocompatible pre-formulation forms a suture. In certain embodiments, the wound patch, joint spacer, or suture gels at least in part at the target site in or on the mammal. In some embodiments, the wound patch, joint spacer, or suture polymerizes at least in part at a target site. In some embodiments, the wound patch, joint spacer, or suture adheres at least partially to the target site.

In certain embodiments, the biocompatible pre-formulation is used as a “liquid suture” or as a drug delivery platform to transport medications directly to the targeted site in or on the mammal. In some embodiments the target site is a joint, a wound or a surgical site. In some embodiments, the spreadability, viscosity, optical clarity, and adhesive properties of the biocompatible pre-formulation or hydrogel polymer matrix are optimized to create materials ideal as liquid sutures for the treatment of diseases. In certain embodiments, the gel time is controlled from 50 seconds to 15 minutes. The site can then be treated with a laser as provided herein.

In some embodiments, the biocompatible hydrogel polymer matrix comprises a buffer or culture medium. In some embodiments, the biocompatible hydrogel polymer matrix comprises a buffer and at least one cell. In some embodiments, the culture medium is a buffer. In some embodiments, the culture medium comprises a growth medium. In some embodiments, the culture medium is nutrient rich. In certain embodiments, the culture medium provides nutrients sufficient for cell viability, growth, and/or proliferation. In certain embodiments, culture media include, but are not limited to, DMEM, IMDM, OptiMEM®, AlgiMatrixTM, Fetal Bovine Serum, GS1-R®, G52-M®, iSTEM®, NDiff® N2,NDiff® N2-AF, RHB-A®, RHB-Basal®, RPMI, SensiCell™, GlutaMAXTM, FluoroBrite™, Gibco® TAP, Gibco® BG-11, LB, M9 Minimal, Terrific Broth, 2YXT, MagicMedia™, ImMedia™, SOC, YPD, CSM, YNB, Grace's Insect Media, 199/109 and HamF10/HamF12. In certain embodiments, the cell culture medium may be serum free. In certain embodiments, the culture medium includes additives. In some embodiments, culture medium additives include, but are not limited to, antibiotics, vitamins, proteins, inhibitors, small molecules, minerals, inorganic salts, nitrogen, growth factors, amino acids, serum, carbohydrates, lipids, hormones and glucose. In some embodiments, growth factors include, but are not limited to, EGF, bFGF, FGF, ECGF, IGF-1, PDGF, NGF, TGF-α and TGF-β. In certain embodiments, the culture medium may not be aqueous. In certain embodiments, the non-aqueous culture medium include, but are not limited to, frozen cell stocks, lyophilized medium and agar.

In some embodiments, one or more optional additional components can be incorporated into the biocompatible hydrogel polymer matrix formulation. Provided herein are biocompatible pre-formulations, comprising at least one first compound comprising more than one nucleophilic group, at least one second compound comprising more than one electrophilic group, optionally at least one cell, and optionally additional components. An exemplary additional component is a buffer. In certain embodiments, the cell is a stem cell. In certain embodiments, the additional component is a culture medium. In certain embodiments, the culture medium is nutrient rich. A biocompatible hydrogel polymer matrix is formed following mixing the first compound, the second compound, and the optional at least one cell in the presence of water; wherein the biocompatible hydrogel polymer matrix gels at a target site. In some embodiments a buffer or other additional components may be added to the pre-formulation mix prior to or after biocompatible hydrogel polymer matrix formation. In some embodiments, the first compound and the second compound do not react with the optional at least one cell during formation of the biocompatible hydrogel polymer matrix. In certain embodiments, the biocompatible hydrogel polymer matrix comprises a biocompatible hydrogel scaffold. In certain embodiments, the biocompatible hydrogel scaffold comprises the at least one first compound and the at least one second compound. In certain embodiments, the biocompatible hydrogel scaffold comprises a buffer. In certain embodiments, the biocompatible hydrogel scaffold is fully synthetic.

Provided herein are biocompatible pre-formulations, comprising at least one first compound comprising more than one nucleophilic group, at least one second compound comprising more than one electrophilic group, a buffer, and optionally additional components. An exemplary additional component is at least one cell. In some embodiments, the composition comprises a cellulose polymer, such as HPMC. In some embodiments, the composition comprises a buffer that maintains the pH of the composition at about 7 to about 7.5 In some embodiments, the buffer is a phosphate buffer, such as PBS. In some embodiments, the pH of the composition is about 7.4

In some embodiments, the composition provided herein and throughout can comprise other or additional viscosity enhancers, such as, but not limited to, acacia, agar, alginic acid, bentonite, carbomers, carboxymethylcellulose calcium, carboxymethylcellulose sodium, carrageenan, ceratonia, cetostearyl alcohol, chitosan, colloidal silicon dioxide, cyclomethicone, ethylcellulose, gelatin, glycerin, glyceryl behenate, guar gum, hectorite, hydrogenated vegetable oil type I, hydroxyethyl cellulose, hydroxyethylmethyl cellulose, hydroxypropyl cellulose, hydroxypropyl starch, hydroxypropylmethylcellulose, magnesium aluminum silicate, maltodextrin, methylcellulose, polydextrose, polyethylene glycol, poly(methylvinyl ether/maleic anhydride), polyvinyl acetate phthalate, polyvinyl alcohol, potassium chloride, polyvinylpyrrolidone, propylene glycol alginate, saponite, sodium alginate, sodium chloride, stearyl alcohol, sucrose, sulfobutylether β-cyclodextrin, tragacanth, xanthan gum and mixtures thereof.

In some embodiments, the composition that forms the hydrogel comprises 8-ARM-AA-20K, 8-ARM-NH2-20K, and 4-ARM-SGA-20K. In some embodiments, the composition comprises sodium phosphate, monobasic, anhydride, sodium phosphate, dibasic, anhydride. In some embodiments, the composition comprises hydroxypropyl methylcellulose (HPMC). In some embodiments, the composition is mixed with water (e.g. the liquid component) to form the hydrogel. In some embodiments, the composition is mixed with sodium hyaluronate (e.g. the liquid component) to form the hydrogel.

8-ARM-AA-20K refers to 8-arm PEG Acetate amine (hexaglycerol), or salts thereof, such as a HCl salt, with a molecular weight of 20k. It can be represented by a formula of:

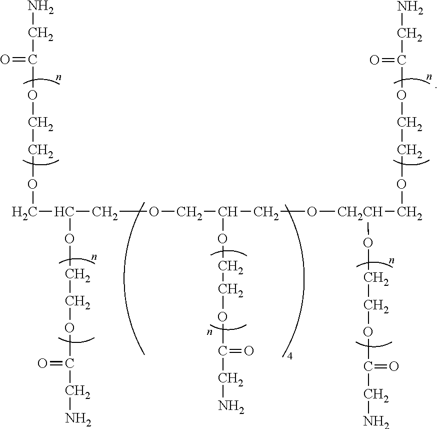

8-ARM-NH2-20K refers to 8-arm PEG amine (hexaglycerol), or salts thereof, such as a HCl salt, with a molecular weight (MW) of 20k. It can be represented by a formula of:

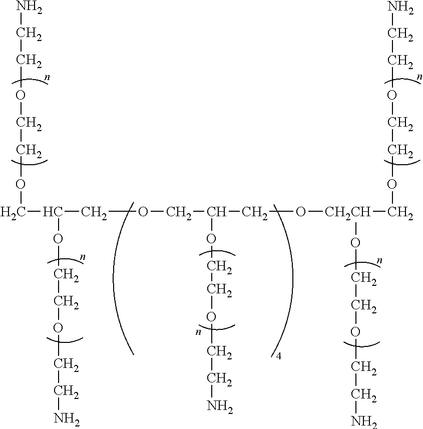

4-ARM-SGA-20K refers to 4-arm PEG succinimidyl glutaramide (pentaerythritol), MW 20k, or salts thereof, such as a HCl salt, with a molecular weight (MW) of 20k. It can be represented by a formula of:

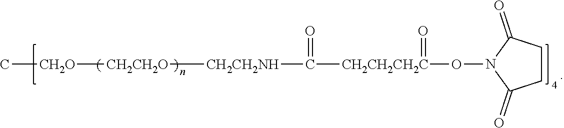

In each of the formulas represented above, each n can independently be 1-200 or 10-200.

In some embodiments, of the compositions provided herein and throughout the ratio of 8ARM-PEG-AA to 8ARM-PEG-NH2 is about 1:1, about 70:30, or about 75:25 (3:1). This can be in mols or by weight. In some embodiments, the 8ARM-PEG-AA, 8ARM-PEG-NH2 and 4ARM-PEG-SGA each have a molecular weight of about 20,000.

In certain embodiments the cell is a stem cell. In certain embodiments, the buffer is a culture medium. In certain embodiments, the culture medium is nutrient rich. A biocompatible hydrogel polymer matrix is formed following mixing the first compound, the second compound, and the buffer in the presence of water; wherein the biocompatible hydrogel polymer matrix gels at a target site. In some embodiments at least one cell or other additional components may be added to the mix prior to or after biocompatible hydrogel polymer matrix formation. In some embodiments, the first compound and the second compound do not react with the optional at least one cell during formation of the biocompatible hydrogel polymer matrix. In certain embodiments, the biocompatible hydrogel polymer matrix comprises a biocompatible hydrogel scaffold. In certain embodiments, the biocompatible hydrogel scaffold comprises the at least one first compound, the at least one second compound and a buffer. In certain embodiments, the biocompatible hydrogel scaffold is fully synthetic.

In certain embodiments, the biocompatible pre-formulation or biocompatible hydrogel polymer matrix comprises at least one additional component. Additional components include, but are not limited to, proteins, biomolecules, growth factors, anesthetics, antibacterials, antivirals, immunosuppressants, anti-inflammatory agents, anti-proliferative agents, anti-angiogenesis agents and hormones.

In some embodiments, the biocompatible hydrogel polymer matrix or biocompatible pre-formulation further comprise a visualization agent for visualizing the placement of the biocompatible hydrogel polymer matrix at a target site The visualization agent assists in visualizing the placement using minimally invasive delivery, e.g., using an endoscopic device. In certain embodiments, the visualization agent is a dye. In specific embodiments, the visualization agent is a colorant.

In some embodiments, the biocompatible hydrogel polymer matrix formulations further comprise a contrast agent for visualizing the biocompatible hydrogel formulation and locating a tumor using e.g., X-ray, fluoroscopy, or computed tomography (CT) imaging. In certain embodiments, the contrast agent is radiopaque. In some embodiments, the radiopaque material is selected from sodium iodide, potassium iodide, barium sulfate, VISIPAQUE®, OMNIPAQUE®, or HYPAQUE®, tantalum, and similar commercially available compounds, or combinations thereof.

### EXAMPLES

The following specific examples are to be construed as merely illustrative, and not limitative of the remainder of the disclosure in any way whatsoever.

The following are general characteristics of the biocompatible pre-formulations and biocompatible hydrogel polymer matrices consistent with biocompatibility.

| | Pre-formulations | |
|---|---|---|
| | Property | Characteristics |
| 1 | In vivo polymerizable | Could be polymerized inside |
| | | mammalian cavity or over |
| | | the skin |
| 2 | Reaction mixture pH | Physiological to 8.0 pH range |
| 3 | Reaction temperature | Ambient to body temperature |
| 4 | Formulation | Two or three component |
| | physical form | system; Mixed immediately |
| | | prior to use, may contain |
| | | radiopaque agent such as |
| | | barium sulphate or iodine |
| | | containing organic compounds |
| | | or other known radiopaque agents |
| 5 | Mixing time for the | Few seconds (~10 sec) |
| | reaction to start | |
| 6 | Gel formation time | Gel formation time ranges |
| | | from 10 seconds to 120 |
| | | seconds, or could be as long |
| | | as 30 minutes depending on |
| | | the application |
| 7 | Solution viscosity | Solution viscosity ranges |
| | | from 1 to 800 cps |
| 8 | Sterilization capability | ETO to E-beam sterilizable |
| 9 | | Ideal for localized delivery for |
| | | small molecules, large |
| | | Localized delivery |
| | | molecules and cells |
| 10 | Stability of drugs in | All small molecule drugs and |
| | formulation mixture | proteins studied so far have |
| | | been found to be stable |

The following are some characteristics of adhesive biocompatible hydrogel polymer matrices.

| | Hydrogel Property | Characteristics |
|---|---|---|
| 1 | Tissue adhesion | Sticky formulations, physicochemical |
| | | characteristics ideal |
| | | for bonding to skin, bones, or |
| | | other mammalian tissues |
| 2 | Polymer hardness | Can be controlled from soft |
| | | tissues to harder cartilage like |
| | | materials |
| 3 | Bioabsorption Time | About 2 weeks up to 10 years, |
| | | or totally non-bioabsorbable |
| 4 | Biocompatibility | Highly biocompatible; passed |
| | | all the subjected ISO 10993 |
| | | tests |
| 5 | Polymer cytotoxicity | Non-cytotoxic formulations |
| 6 | Small molecule elution | Small drug molecules elution |
| | | can be controlled and thus |
| | | pharmaceutical drugs could |
| | | also be delivered using the |
| | | formulations, if needed |
| 7 | Compatibility with | Highly compatible due to |
| | proteins and Cells | physiological pH of the polymers |

Biocompatible pre-formulation chemical components used to form biocompatible hydrogel polymer matrices are listed in Table 1. These biocompatible pre-formulation components will be referred to by their abbreviations. Several USP grade viscosity enhancing agents were purchased from Sigma-Aldrich and were stored at 25° C. They include methylcellulose (Methocel® MC, 10-25MPA·S) abbreviated as MC; hypromellose (hydroxypropylmethylcellulose 2910) abbreviated as HPMC; and povidone K-30 (polyvinylpyrrolidone) abbreviated as PVP.

The biocompatible pre-formulation components were stored at 5° C. and allowed to warm to room temperature before use, which typically took 30 minutes. After use the contents were purged with N2 for approximately 30 seconds before sealing with parafilm and returning to 5° C. Alternately, the biocompatible pre-formulation components were stored at −20° C. and allowed to warm to room temperature before use under the flow of inert gas, which typically took 30 minutes. The biocompatible pre-formulation components were purged with inert gas for at least 30 seconds before returning to −20° C.

A 0.15 M phosphate buffer was made by dissolving 9.00 g (0.075 mol) NaH₂PO₄ in 500 mL of distilled water at 25° C. with magnetic stirring. The pH was then adjusted to 7.99 with the dropwise addition of 50% aqueous NaOH. Several other phosphate buffers were prepared in a similar fashion: 0.10 M phosphate at pH 9, 0.10 M phosphate at pH 7.80, 0.10 M phosphate at 7.72, 0.10 M phosphate at pH 7.46, 0.15 M phosphate at pH 7.94, 0.15 M phosphate at pH 7.90, 0.4 M phosphate at pH 9, and 0.05 M phosphate at pH 7.40.

A sterile 0.10 M phosphate buffer at pH 7.58 with 0.30% HPMC was prepared for use in kits. First, 1.417 g HPMC was dissolved in 471 mL of 0.10 M phosphate buffer at pH 7.58 by vigorous shaking. The viscous solution was allowed to clarify overnight. The solution was filtered through a 0.22 μm filter (Corning #431097) with application of light vacuum. The viscosity of the resulting solution was measured to be 8.48 cSt+/−0.06 at 20° C.

A sterile 0.10 M phosphate buffer at pH 7.58 with 0.3% HPMC was prepared. First, a 0.10 M phosphate buffer was made by dissolving 5.999 g (0.05 mol) of NaH₂PO₄ in 500 mL of distilled water at 20° C. with magnetic stirring. The pH was then adjusted to 7.58 with the dropwise addition of 50% aqueous NaOH. Then, 1.5 g of HPMC was dissolved in 500 mL of the above buffer solution by vigorous shaking. The viscous solution was allowed to clarify overnight. The solution was filtered through a 0.22 μm filter (Corning #431097) with application of light vacuum. The viscosity of the resulting solution was measured via the procedure as described in the Viscosity Measurements section and was found to be 8.48 cSt+/−0.06 at 20° C.

Phosphate buffered saline (PBS) was prepared by dissolving two PBS tablets (Sigma Chemical, P4417) in 400 mL of distilled water at 25° C. with vigorous shaking. The solution has the following composition and pH: 0.01 M phosphate, 0.0027 M potassium chloride, 0.137 M sodium chloride, pH 7.46.

A 0.058 M phosphate buffer was made by dissolving 3.45 g (0.029 mol) of NaH₂PO₄ in 500 mL of distilled water at 25° C. with magnetic stirring. The pH was then adjusted to 7.97 with the dropwise addition of 50% aqueous NaOH.

A 0.05 M borate buffer was made by dissolving 9.53 g (0.025 mol) of Na₂B₄O₇.10H₂O in 500 mL of distilled water at 25° C. with magnetic stirring. The pH was then adjusted to 7.93 or 8.35 with the dropwise addition of 6.0 N HCl.

An antiseptic liquid component was prepared in a similar fashion with a commercial 2% chlorhexidine solution. To 100 mL of 2% chlorhexidine solution was dissolved 0.3 g of HPMC. The viscous solution was allowed to clarify overnight at 5° C. The resulting clear blue solution has the following composition: 2% chlorhexidine, 0.3% HPMC and an unknown quantity of nontoxic blue dye and detergent.

Other liquid components were prepared in a similar fashion by simply dissolving the appropriate amount of the desired additive to the solution. For example, an antiseptic liquid component with 1% denatonium benzoate, a bittering agent, was prepared by dissolving 2 g of denatonium benzoate in 200 mL of 2% chlorhexidine solution.

Alternatively, commercially available drug solutions were used as the liquid component. For example, saline solution, Kenalog-10 (10 mg/mL solution of triamcinolone acetonide) and Depo-Medrol (40 mg/mL of methylprednisolone acetate) were used.

The amine or thiol component (typically in the range of 0.1 mmol arms equivalents) was added to a 50 mL centrifuge tube. A volume of reaction buffer was added to the tube via a pipette such that the final concentration of solids in solution was about 5 percent. The mixture was gently swirled to dissolve the solids before adding the appropriate amount of ester or epoxide. Immediately after adding the ester or epoxide, the entire solution was shaken for 10 seconds before letting it rest.

TABLE 1
Components used in biocompatible_pre-formulations.

| Pre-formulation | |
|---|---|
| Components | Technical Name |
| ETTMP-1300 | Ethoxylated trimethylolpropane |
| | tri(3-mercaptopropionate) |
| 4ARM-5k-SH | 4ARM PEG Thiol (pentaerythritol) |
| 4ARM-2k-NH2 | 4ARM PEG Amine (pentaerythritol), |
| | HCl Salt, MW 2000 |
| 4ARM-5k-NH2 | 4ARM PEG Amine (pentaerythritol), |
| | HCl Salt, MW 5000 |
| 8ARM-20k-NH2 | 8ARM PEG Amine (hexaglycerol), |
| | HCl Salt, MW 20000 |
| 4ARM-20k-AA | 4ARM PEG Acetate Amine |
| | HCl Salt, MW 20000 |
| 8ARM-20k-AA | 8ARM PEG Acetate Amine |
| | (hexaglycerol) HCl Salt, MW 20000 |
| 8ARM-20k-AA | 8ARM PEG Acetate Amine |
| | (hexaglycerol) TFA Salt, MW 20000 |
| 4ARM-10k-SG | 4ARM PEG Succinimidyl Glutarate |
| | (pentaerythritol), MW 10000 |
| 8ARM-15k-SG | 8ARM PEG Succinimidyl Glutarate |
| | (hexaglycerol), MW 15000 |
| 4ARM-20k-SGA | 4ARM PEG Succinimidyl Glutaramide |
| | (pentaerythritol), MW 20000 |
| 4ARM-10k-SS | 4ARM PEG Succinimidyl Succinate |
| | (pentaerythritol), MW 10000 |
| EJ-190 | Sorbitol polyglycidyl ether |
| MC | Methyl Cellulose (Methocel ® MC) |
| HPMC | Hypromellose (Hydroxypropylmethylcellulose) |
| PVP | Povidone (polyvinylpyrrolidone) |

The gel time for all cases was measured starting from the addition of the ester or epoxide until the gelation of the solution. The gel point was noted by pipetting 1 mL of the reaction mixture and observing the dropwise increase in viscosity. Degradation of the polymers was performed by the addition of 5 to 10 mL of phosphate buffered saline to ca. 5 g of the material in a 50 mL centrifuge tube and incubating the mixture at 37° C. The degradation time was measured starting from the day of addition of the phosphate buffer to complete dissolution of the polymer into solution.

### Example 1: Manufacture of a Biocompatible Hydrogel Polymer Matrix (Amine-Ester Chemistry)

A solution of 8ARM-20K-NH2 was prepared in a Falcon tube by dissolving about 0.13 g solid monomer in about 2.5 mL of sodium phosphate buffer (buffer pH 7.36). The mixture was shaken for about 10 seconds at ambient temperature until complete dissolution was obtained. The Falcon tube was allowed to stand at ambient temperature. In another Falcon tube, 0.10 g of 8ARM-15K-SG was dissolved in the same phosphate buffer as above. The mixture was shaken for about 10 seconds and at this point all the powder dissolved. The 8ARM-15K-SG solution was poured immediately into the 8ARM-20K-NH2 solution and a timer was started. The mixture was shaken and mixed for about 10 seconds and a 1 mL solution of the mixture was pipetted out using a mechanical high precision pipette. The gel time of 1 mL liquid was collected and then verified with the lack of flow for the remaining liquids. The gel time data of the formulation was recorded and was about 90 seconds.

### Example 2: Manufacture of a Biocompatible Hydrogel Polymer Matrix (Amine-Ester Chemistry)

A solution of amines was prepared in a Falcon tube by dissolving about 0.4 g solid 4ARM-20k-AA and about 0.2 g solid 8ARM-20k-NH2 in about 18 mL of sodium phosphate buffer (buffer pH 7.36). The mixture was shaken for about 10 seconds at ambient temperature until complete dissolution was obtained. The Falcon tube was allowed to stand at ambient temperature. To this solution, 0.3 g of 8ARM-15K-SG was added. The mixture was shaken to mix for about 10 seconds until all the powder dissolved. 1 mL of the mixture was pipetted out using a mechanical high precision pipette. The gel time of the formulation was collected using the process described above. The gel time was about 90 seconds.

### Example 3: Manufacture of a Biocompatible Hydrogel Polymer Matrix (Thiol-Ester Chemistry)

A solution of ETTMP-1300 was prepared in a Falcon tube by dissolving about 0.04 g monomer in about 5 mL of sodium borate buffer (buffer pH 8.35). The mixture was shaken for about 10 seconds at ambient temperature until complete dissolution was obtained. The Falcon tube was allowed to stand at ambient temperature. To this solution, 0.20 g of 8ARM-15K-SG was added. The mixture was shaken for about 10 seconds until the powder dissolved. 1 mL of the mixture was pipetted out using a mechanical high precision pipette. The gel time was found to be about 70 seconds.

### Example 4: Manufacture of a Biocompatible Hydrogel Polymer Matrix (Thiol-Epoxide Chemistry)

A solution of ETTMP-1300 was prepared in a Falcon tube by dissolving about 0.04 g monomer in about 5 mL of sodium borate buffer (buffer pH 8.35). The mixture was shaken for about 10 seconds at ambient temperature until complete dissolution was obtained. The Falcon tube was allowed to stand at ambient temperature. To this solution, 0.10 g of EJ-190 was added. The mixture was shaken for about 10 seconds until complete dissolution is obtained. 1 mL of the mixture was pipetted out using a mechanical high precision pipette. The gel time was found to be about 6 minutes.

### Example 5: In Vitro Bioabsorbance Testing

A 0.10 molar buffer solution of pH 7.40 was prepared with deionized water. A 50 mL portion of this solution was transferred to a Falcon tube. A sample polymer was prepared in a 20 cc syringe. After curing, a 2-4 mm thick slice was cut from the polymer slug and was placed in the Falcon tube. A circulating water bath was prepared and maintained at 37° C. The Falcon tube with polymer was placed inside the water bath and time was started. The dissolution of the polymer was monitored and recorded. The dissolution time ranged from 1-90 days depending on the type of sample polymer.

### Example 6: Gelling and Degradation Times of Amine-Ester Polymers

Amines studied were 8ARM-20k-NH2 and 4ARM-5k-NH2. The formulation details and material properties are given in Table 2. With 8ARM-20k-NH2, it was found that a phosphate buffer with 0.058 M phosphate and pH of 7.97 was necessary to obtain acceptable gel times of around 100 seconds. Using a 0.05 M phosphate buffer with a pH of 7.41 resulted in a more than two-fold increase in gel time (270 seconds).

With the 8ARM-20k-NH2, the ratio of 4ARM-10k-SS to 4ARM-20k-SGA was varied from 50:50 to 90:10. The gel time remained consistent, but there was a marked shift in degradation time around a ratio of 80:20. For formulations with ratios of 75:25 and 50:50, degradation times spiked to one month and beyond. Using lower amounts of 4ARM-20k-SGA (80:20, 85:15, 90:10) resulted in degradation times of less than 7 days.

As a comparison, the 4ARM-5k-NH2 was used in a formulation with a ratio of 4ARM-10k-SS to 4ARM-20k-SGA of 80:20. As was expected, the degradation time remained consistent, which suggests that the mechanism of degradation was unaffected by the change in amine. However, the gel time increased by 60 seconds, which may reflect the relative accessibility of reactive groups in a high molecular weight 8ARM amine and a low molecular weight 4ARM amine.

TABLE 2
Gel and degradation times for varying 4ARM-10k-SS/4ARM-20k-SGA
ratios with 8ARM-15k-SG ester.

| | | Phosphate | | |
|---|---|---|---|---|
| | | Reaction | | |
| | Ratio of | Buffer | | |
| | 4ARM- | Con- | | |
| | 10k-SS/ | centration | Gel | Degradation |
| Pre-formulation | 4ARM-20k- | and | Time | Time |
| Components | SGA | pH | (s) | (days) |
| 8ARM-20k-NH2 | 50/50 | 0.05M | 270 | N/A |
| 4ARM-10k-SS, | | pH 7.41 | | |
| 4ARM-20k-SGA | | | | |
| 8ARM-20k-NH2 | 50/50 | 0.058M | 100 | >41 |
| 4ARM-10k-SS, | | pH 7.97 | | |
| 4ARM-20k-SGA | | | | |
| 8ARM-20k-NH2 | 75/25 | 0.058M | 90 | 29 |
| 4ARM-10k-SS, | | pH 7.97 | | |
| 4ARM-20k-SGA | | | | |
| 8ARM-20k-NH2 | 80/20 | 0.058M | 100 | 7 |
| 4ARM-10k-SS, | | pH 7.97 | | |
| 4ARM-20k-SGA | | | | |
| 4ARM-5k-NH2 | 80/20 | 0.058M | 160 | 6 |
| 4ARM-10k-SS, | | pH 7.97 | | |
| 4ARM-20k-SGA | | | | |
| 8ARM-20k-NH2 | 85/15 | 0.058M | 100 | 5 |
| 4ARM-10k-SS, | | pH 7.97 | | |
| 4ARM-20k-SGA | | | | |
| 8ARM-20k-NH2 | 90/10 | 0.058M | 90 | 6 |
| 4ARM-10k-SS, | | pH 7.97 | | |
| 4ARM-20k-SGA | | | | |

### Example 7: Gelling and Degradation Times of Thiol-Ester Polymers

Thiols studied were 4ARM-5k-SH and ETTMP-1300. The formulation details and material properties are given in Table 3. It was found that a 0.05 M borate buffer with a pH of 7.93 produced gel times of around 120 seconds. Increasing the amount of 4ARM-20k-SGA in the formulation increased the gel time to 190 seconds (25:75 ratio of 4ARM-10k-SS to 4ARM-20k-SGA) up to 390 seconds (0:100 ratio of 4ARM-10k-SS to 4ARM-20k-SGA). Using a 0.05 M borate buffer with a pH of 8.35 resulted in a gel time of 65 seconds, about a two-fold decrease in gel time. Thus, the gel time may be tailored by simply adjusting the pH of the reaction buffer.

The ratio of 4ARM-10k-SS to 4ARM-20k-SGA was varied from 0:100 to 100:0. In all cases, the degradation time did not vary significantly and was typically between 3 and 5 days. It is likely that degradation is occurring via alternate pathways.

TABLE 3
Gel and degradation times for varying 4ARM-10k-SS/4ARM-20k-SGA
ratios with 4ARM-5k-SH and ETTMP-1300 thiols.

| | Ratio of | Phosphate | | |
|---|---|---|---|---|
| | 4ARM- | Reaction | | |
| | 10k-SS/ | Buffer | | |
| | 4ARM- | Concentration | Gel | Degradation |
| Pre-formulation | 20k- | and | Time | Time |
| Components | SGA | pH | (s) | (days) |
| 4ARM-5k-SH | 50/50 | 0.05M | 65 | N/A |
| 4ARM-10k-SS, | | pH 8.35 | | |
| 4ARM-20k-SGA | | | | |
| 4ARM-5k-SH | 50/50 | 0.05M | 120 | 4 |
| 4ARM-10k-SS, | | pH 7.93 | | |
| 4ARM-20k-SGA | | | | |
| 4ARM-5k-SH | 75/25 | 0.05M | 125 | 4 |
| 4ARM-10k-SS, | | pH 7.93 | | |
| 4ARM-20k-SGA | | | | |
| 4ARM-5k-SH | 90/10 | 0.05M | 115 | 4 |
| 4ARM-10k-SS, | | pH 7.93 | | |
| 4ARM-20k-SGA | | | | |
| 4ARM-5k-SH | 25/75 | 0.05M | 190 | 4 |
| 4ARM-10k-SS, | | pH 7.93 | | |
| 4ARM-20k-SGA | | | | |
| 4ARM-5k-SH | 10/90 | 0.05M | 200 | 4 |
| 4ARM-10k-SS, | | pH 7.93 | | |
| 4ARM-20k-SGA | | | | |
| ETTMP-1300 | 0/100 | 0.05M | 390 | 3 |
| 4ARM-20k-SGA | | | | |
| 4ARM-5k-SH | 100/0 | 0.05M | 120 | 4 |
| 4ARM-10k-SS | | pH 7.93 | | |

### Example 8: Gelling and Degradation Times of Amine-Ester and Thiol-Ester Polymers

An amine (4ARM-5k-NH2) and a thiol (4ARM-5k-SH) were studied with the ester 4ARM-10k-SG. The formulation details and material properties are given in Table 4. A 0.058 M phosphate buffer with a pH of 7.97 yielded a gel time of 150 seconds with the amine. A 0.05 M borate buffer with a pH of 8.35 produced a gel time of 75 seconds with the thiol.

The amine-based polymer appeared to show no signs of degradation, as was expected from the lack of degradable groups. However, the thiol-based polymer degraded in 5 days. This suggests that degradation is occurring through alternate pathways, as was observed in the thiol formulations with 4ARM-10k-SS and 4ARM-20k-SGA (vida supra).

TABLE 4
Gel and degradation times for amines and thiols with 4ARM-10k-SG
biocompatible pre-formulations.

| Pre-formulation | Reaction Buffer Type, | Gel Time | Degradation |
|---|---|---|---|
| Components | Concentration, and pH | (s) | Time (days) |
| 4ARM-5k-NH2 & | Phosphate (0.058M, | 150 | Indefinite |
| 4ARM-10k-SG | pH 7.97) | | |
| 4ARM-5k-SH & | Borate (0.05M, pH 8.35) | 75 | 5 |
| 4ARM-10k-SG | | | |

### Example 9: Gelling and Degradation Times of Thiol-Sorbitol Polyglycidyl Ether Polymers

With ETTMP-1300 conditions such as high pH (10), high solution concentration (50%), or high borate concentration (0.16 M) were necessary for the mixture to gel. Gel times ranged from around 30 minutes to many hours. The conditions that were explored include: pH from 7 to 12; solution concentration from 5% to 50%; borate concentration from 0.05 M to 0.16 M; and thiol to epoxide ratios from 1:2 to 2:1.

The high pH necessary for the reaction to occur could result in degradation of the thiol. Thus, a polymer with EJ-190 and 4ARM-5k-SH was prepared. A 13% solution formulation exhibited a gel time of 230 seconds at a pH of between 9 and 10. The degradation time was 32 days. At a lower pH of around 8, the mixture exhibited gel times in the range of 1 to 2 hours.

### Example 10: General Procedure for the Preparation of Polymerizable Biocompatible Pre-Formulations

Several representative sticky formulations are listed in Table 5 along with specific reaction details for the preparation of polymerizable biocompatible pre-formulations. The biocompatible hydrogel polymers were prepared by first dissolving the amine component in phosphate buffer or the thiol component in borate buffer. The appropriate amount of the ester component was then added and the entire solution was mixed vigorously for 10 to 20 seconds. The gel time was measured starting from the addition of the ester until the gelation of the solution.

TABLE 5
(A) Summary of the reaction details for several representative sticky formulations without
viscosity enhancer; (B) more detailed tabulation of a selection of the reaction details including
moles (degradation times were measured in phosphate buffered saline (PBS) at 37° C.).
(A)

| | Amine or | | | | |
|---|---|---|---|---|---|
| | Thiol/ | | | | |
| | Ester | | | Gel | |
| Pre-formulation | Molar | | % | Time | Degradation |
| Components | Ratio | Buffer | Solution | (s) | Time (days) |
| 8ARM-20k-NH2 | 3 | 0.15M phosphate, | 3 | 130 | N/A |
| 4ARM-20K-SGA | | pH 7.99 | | | |
| 8ARM-20k-NH2 | 1/3 | 0.15M phosphate, | 3 | 300 | N/A |
| 4ARM-20K-SGA | | pH 7.99 | | | |
| 8ARM-20k-NH2 | 3 | 0.15M phosphate, | 8 | 50 | N/A |
| 4ARM-10K-SS | | pH 7.99 | | | |
| 8ARM-20k-NH2 | 1/3 | 0.15M phosphate, | 8 | 80 | N/A |
| 4ARM-10K-SS | | pH 7.99 | | | |
| 4ARM-20K-AA/ | 3 | 0.15M phosphate, | 5 | 210 | 1 to 3 |
| 8ARM-20k-NH2 | | pH 7.99 | | | |
| (75/25) | | | | | |
| 4ARM-20K-SGA | | | | | |
| 4ARM-20K-AA/ | 5 | 0.15M phosphate, | 10 | 180 | 1 to 3 |
| 8ARM-20k-NH2 | | pH 7.99 | | | |
| (75/25) | | | | | |
| 4ARM-20K-SGA | | | | | |
| 4ARM-5K-NH2 | 5 | 0.10M phosphate, | 10 | 160 | 7 |
| 4ARM-10K-SG | | pH 7.80 | | | |
| 4ARM-5K-NH2 | 5 | 0.10M phosphate, | 20 | 160 | 1 to 3 |
| 4ARM-10K-SS | | pH 7.80 | | | |
| 4ARM-5K-NH2 | 3 | 0.10M phosphate, | 5 | 160 | 13 |
| 4ARM-10K-SG | | pH 7.80 | | | |
| 4ARM-5K-NH2 | 5 | 0.15M phosphate, | 20 | 80 | 7 |
| 4ARM-10K-SG | | pH 7.99 | | | |
| 4ARM-5K-NH2 | 5 | 0.15M phosphate, | 30 | 70 | 10 |
| 4ARM-10K-SG | | pH 7.99 | | | |
| 4ARM-5K-NH2 | 5 | 0.15M phosphate, | 19 | 60 | 53 |
| 4ARM-20K-SGA | | pH 7.99 | | | |
| 4ARM-5K-NH2 | 5 | 0.15M phosphate, | 12 | 70 | 53 |
| 4ARM-20K-SGA | | pH 7.99 | | | |
| 4ARM-5K-NH2 | 1/5 | 0.15M phosphate, | 19 | 160 | 15 |
| 4ARM-10K-SG | | pH 7.99 | | | |
| 4ARM-SH-5K | 5 | 0.05M borate, | 20 | 120 | 2 to 4 |
| 4ARM-10K-SG | | pH 7.93 | | | |
| 4ARM-NH2-2K | 5 | 0.10M phosphate, | 10 | 120 | 15 |
| 8ARM-15K-SG | | pH 7.46 | | | |
| 4ARM-NH2-2K | 7 | 0.10M phosphate, | 30 | 150 | N/A |
| 4ARM-20K-SGA | | pH 7.80 | | | |

(B)

| | | | | | | | Polymer |
|---|---|---|---|---|---|---|---|
| Pre-formulation | | | Wt | | | Arms | % Solution |
| Components | MW | Mmoles | (g) | Arm | mmoles | Eq | (w/v) |
| 8ARM-20k-NH2 | 20000 | 1000 | 0.075 | 8 | 0.00375 | 0.03 | |
| 4ARM-20k-SGA | 20000 | 1000 | 0.05 | 4 | 0.0025 | 0.01 | |
| Buffer Volume (phosphate) | | | 4.1 | | | | 3.0 |
| 8ARM-20k-NH2 | 20000 | 1000 | 0.025 | 8 | 0.00125 | 0.01 | |
| 4ARM-20k-SGA | 20000 | 1000 | 0.15 | 4 | 0.0075 | 0.03 | |
| Buffer Volume (phosphate) | | | 5.8 | | | | 3.0 |
| 8ARM-20k-NH2 | 20000 | 1000 | 0.3 | 8 | 0.015 | 0.12 | |
| 4ARM-10k-SS | 10000 | 1000 | 0.1 | 4 | 0.01 | 0.04 | |
| Buffer Volume (phosphate) | | | 5 | | | | 8.0 |
| 8ARM-20k-NH2 | 20000 | 1000 | 0.1 | 8 | 0.005 | 0.04 | |
| 4ARM-10k-SS | 10000 | 1000 | 0.3 | 4 | 0.03 | 0.12 | |
| Buffer Volume (phosphate) | | | 5 | | | | 8.0 |

TABLE 6
Gel times for the 8ARM-20k-NH2/4ARM-20k-SGA(1/1) sticky
polymers including HPMC as viscosity enhancer with
varying buffers and concentrations.

| Pre-formulation | Amine/Ester | | % | Gel |
|---|---|---|---|---|
| Components | Molar Ratio | Buffer | Solution | Time (min) |
| 8ARM-20k-NH2 | 1 | 0.10M | 4.8 | 1.5 |
| 4ARM-20K-SGA | | phosphate, | | |
| 0.3% HPMC | | pH 7.80 | | |
| 8ARM-20k-NH2 | 1 | 0.10M | 4.8 | 3.5 |
| 4ARM-20K-SGA | | phosphate, | | |
| 0.3% HPMC | | pH 7.46 | | |
| 8ARM-20k-NH2 | 1 | 0.05M | 4.8 | 4.5 |
| 4ARM-20K-SGA | | phosphate, | | |
| 0.3% HPMC | | pH 7.42 | | |
| 8ARM-20k-NH2 | 1 | 0.05M | 4 | 5.5 |
| 4ARM-20K-SGA | | phosphate, | | |
| 0.3% HPMC | | pH 7.42 | | |
| 8ARM-20k-NH2 | 1 | 0.05M | 3 | 8.5 |
| 4ARM-20K-SGA | | phosphate, | | |
| 0.3% HPMC | | pH 7.42 | | |
| 8ARM-20k-NH2 | 1 | 0.05M | 4.8 | 6.75 |
| 4ARM-20K-SGA | | phosphate, | | |
| 0.3% HPMC | | pH 7.24 | | |
| 8ARM-20k-NH2 | 1 | 0.05M | 3 | 12 |
| 4ARM-20K-SGA | | phosphate, | | |
| 0.3% HPMC | | pH 7.24 | | |
| 8ARM-20k-NH2 | 1 | 0.05M | 2.5 | 15.5 |
| 4ARM-20K-SGA | | phosphate, | | |
| 0.3% HPMC | | pH 7.24 | | |

Gel times ranged from 60 to 300 seconds and were found to be easily tuned by adjusting the reaction buffer pH, buffer concentration, or polymer concentration. An example of gel time control for a single formulation is shown in Table 6, where the gel time for the 8ARM-20k-NH2/4ARM-20k-SGA (1/1) polymer was varied from 1.5 to 15.5 minutes.

In some instances, the stickiness of the polymers originates from a mismatching in the molar equivalents of the components. A variety of sticky materials using combinations of 4 or 8 armed amines of molecular weights between 2 and 20 thousand and 4 or 8 armed esters of molecular weights between 10 and 20 thousand were created. It was found that in comparison with the 8 armed esters, the 4 armed esters resulted in stickier materials. For the amine component, it was found that smaller molecular weights led to stickier materials and higher amine to ester molar ratios.

A mismatch (amine to ester molar ratio) of at least 3 was required to qualitatively sense stickiness. More preferably, a ratio of around 5 produced a desirable level of stickiness combined with polymer strength. Polymers with amine to ester molar ratios higher than 5 may be formed as well, but some reaction conditions, such as the polymer concentration, may need to be adjusted to obtain a reasonable gel time. Furthermore, it was found that the use of a viscosity enhanced solution improves the polymers by increasing their strength and elasticity, allowing for higher amine to ester molar ratios (Example 11; Table 9).

The materials formed were typically transparent and elastic. Stickiness was tested for qualitatively by touch. Thus, a sticky material adhered to a human finger or other surface and remained in place until removed. Degradation times varied from 1 to 53 days. In certain instances, the polymer properties, such as gel and degradation times, pore sizes, swelling, etc. may be optimized for different applications without losing the stickiness.

### Example 11: General Procedure for the Preparation of Solutions with Enhanced Viscosity

Polymer solutions with enhanced viscosities were prepared by the addition of a viscosity enhancing agent to the reaction buffer. Table 9B lists the viscosity enhancing agents studied, including observations on the properties of the formed polymers. Stock solutions of reaction buffers were prepared with varying concentrations of methylcellulose (MC), hypromellose (HPMC) or polyvinylpyrrolidone (PVP). As an example, a 2% (w/w) HPMC solution in buffer was made by adding 0.2 g of HPMC to 9.8 mL of 0.10 M phosphate buffer at pH 7.80, followed by vigorous shaking. The solution was allowed to stand overnight. Buffer solutions with HPMC concentrations ranging from 0.01% to 2.0% were prepared in a similar fashion. Buffer solutions with PVP concentrations ranging from 5% to 20% and buffer solutions with MC concentrations ranging from 1.0 to 2.0% were also prepared by a similar method.

The polymers were formed in the same method as described above in the general procedures for the preparation of the sticky materials (Example 10). A typical procedure involved first dissolving the amine component in the phosphate buffer containing the desired concentration of viscosity enhancing agent. The appropriate amount of the ester component was then added and the entire solution was mixed vigorously for 10 to 20 seconds. The gel time was measured starting from the addition of the ester until the gelation of the solution.

Several representative formulations are listed in Table 7 and Table 8 along with specific reaction details. The percent of degradable acetate amine component by mole equivalents is represented by a ratio designated in parenthesis. For example, a formulation with 75% degradable amine will be written as 8ARM-20k-AAI8ARM-20k-NH2 (75/25). The polymer was prepared by first dissolving the formulation amine component in phosphate buffer. The appropriate amount of the formulation ester component was then added and the entire solution was mixed vigorously for 10 to 20 seconds. The gel time was measured starting from the addition of the ester until the gelation of the solution.

The gel time is dependent on several factors: pH, buffer concentration, polymer concentration, temperature and the biocompatible pre-formulation monomers used. Previous experiments have shown that the extent of mixing has little effect on the gel time once the components are in solution, which typically takes up to 10 seconds. The effect of biocompatible pre-formulation monomer addition on buffer pH was measured. For the 8ARM-20k-NH2 & 4ARM-20k-SGA formulation, the buffer pH drops slightly from 7.42 to 7.36 upon addition of the biocompatible pre-formulation monomers. For the 8ARM-20k-AAI8ARM-20k-NH2 (70/30) & 4ARM-20k-SGA formulation, the buffer pH drops from 7.4 to 7.29 upon addition of the biocompatible pre-formulation monomers. The additional decrease in the pH was found to originate from acidic residues in the degradable acetate amine. The same pH drop phenomenon was observed for the 4ARM-20k-AA amine. In certain instances, a quality control specification on the acetate amine solution pH may be required to improve the consistency of degradable formulations.

The effect of reaction buffer pH on gel times was measured. The gel times increase with an increase in the concentration of hydronium ions in an approximately linear fashion. More generally, the gel times decrease with an increase in the buffer pH. In addition, the effect of reaction buffer phosphate concentration on gel times was determined. The gel times decrease with an increase in the phosphate concentration. Furthermore, the effect of polymer concentration on gel times was investigated. The gel times decrease significantly with an increase in the polymer concentration. At low polymer concentrations where the gel time is greater than 5 minutes, hydrolysis reactions of the ester begin to compete with the formation of the polymer. The effect of temperature on gel times appears to follow the Arrhenius equation. The gel time is directly related to the extent of reaction of the polymer solution and so this behavior is not unusual.

The rheology of the polymers during the gelation process as a function of the percent time to the gel point was determined. When 100% represents the gel point and 50% represents half the time before the gel point, the viscosity of the reacting solution remains relatively constant until about 80% of the gel point. After that point, the viscosity increases dramatically, representing the formation of the solid gel.

The gel time stability of a single formulation using the same lot of biocompatible pre-formulation monomers over the course of about a year was measured. The biocompatible pre-formulation monomers were handled according to the standard protocol outlined above. The gel times remained relatively stable; some variations in the reaction buffer may account for differences in the gel times.

TABLE 7
(A) Summary of the reaction details for several representative sticky formulations;
(B) more detailed tabulation of a selection of the reaction details including moles
(degradation times were measured in phosphate buffered saline (PBS) at 37° C.).
(A)

| | | % | Gel | Degradation |
|---|---|---|---|---|
| Pre-formulation Components | Buffer | Solution | Time (s) | Time (days) |
| 4ARM-20k-AA/8ARM-20k-NH2 | 0.10M phosphate, | 5 | 150 | 21 |
| (60/40) | pH 7.80 | | | |
| 4ARM-20k-SGA | | | | |
| 4ARM-20k-AA/8ARM-20k-NH2 | 0.10M phosphate, | 5 | 150 | 21 |
| (60/40) | pH 7.80 | | | |
| 4ARM-20k-SGA | | | | |
| 0.3% HPMC | | | | |
| 8ARM-20k-NH2 | 0.10M phosphate, | 4.8 | 100 | N/A |
| 4ARM-20k-SGA | pH 7.80 | | | |
| 0.3% HPMC | | | | |
| 8ARM-20k-NH2 | 0.10M phosphate, | 4.8 | 70 | 48 |
| 8ARM-15k-SG | pH 7.80 | | | |
| 0.3% HPMC | | | | |
| 4ARM-20k-AA/8ARM-20k-NH2 | 0.10M phosphate, | 4.8 | 110 | 12 |
| (60/40) | pH 7.80 | | | |
| 8ARM-15k-SG | | | | |
| 0.3% HPMC | | | | |
| 4ARM-20k-AA/8ARM-20k-NH2 | 0.10M phosphate, | 20 | 160 | 21 |
| (60/40) | pH 7.80 | | | |
| 4ARM-20k-SGA | | | | |
| 0.3% HPMC | | | | |
| 8ARM-20k-NH2 | 0.10M phosphate, | 4.8 | 90 | N/A |
| 4ARM-20k-SGA | pH 7.80 | | | |
| 8ARM-20k-NH2 | 0.10M phosphate, | 4.8 | 80 | N/A |
| 4ARM-20k-SGA | pH 7.80 | | | |
| 1.0% HPMC | | | | |
| 8ARM-20k-NH2 | 0.10M phosphate, | 4.8 | 210 | N/A |
| 4ARM-20k-SGA | pH 7.46 | | | |
| 0.3% HPMC | | | | |
| 8ARM-20k-NH2 | 0.05M phosphate, | 4.8 | 270 | N/A |
| 4ARM-20k-SGA | pH 7.42 | | | |
| 0.3% HPMC | | | | |
| 8ARM-20k-NH2 | 0.05M phosphate, | 4 | 330 | N/A |
| 4ARM-20k-SGA | pH 7.42 | | | |
| 0.3% HPMC | | | | |
| 8ARM-20k-NH2 | 0.05M phosphate, | 3 | 510 | N/A |
| 4ARM-20k-SGA | pH 7.42 | | | |
| 0.3% HPMC | | | | |
| 8ARM-20k-NH2 | 0.05M phosphate, | 4.8 | 405 | N/A |
| 4ARM-20k-SGA | pH 7.24 | | | |
| 0.3% HPMC | | | | |
| 8ARM-20k-NH2 | 0.05M phosphate, | 3 | 720 | N/A |
| 4ARM-20k-SGA | pH 7.24 | | | |
| 0.3% HPMC | | | | |
| 8ARM-20k-NH2 | 0.05M phosphate, | 2.5 | 930 | N/A |
| 4ARM-20k-SGA | pH 7.24 | | | |
| 0.3% HPMC | | | | |
| 8ARM-20k-AA | 0.10M phosphate, | 4.8 | 90 | 6 |
| 4ARM-20k-SGA | pH 7.46 | | | |
| HPMC (0.3%) | | | | |
| 8ARM-20k-AA/8ARM-20k-NH2 | 0.10M phosphate, | 4.8 | 100 | 16 |
| (75/25) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 95 | 256 |
| (60/40) | pH 7.46 | | | (estimated) |
| 4ARM-20k-SGA | | | | |
| HPMC (0.3%) | | | | |
| 8ARM-20k-AA/8ARM-20k-NH2 | 0.10M phosphate, | 4.8 | 120 | N/A |
| (50/50) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 100 | 21 |
| (70/30) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 100 | 28 |
| (65/35) | pH 7.46 | | | |
| 4ARM-20k-SGA | | | | |
| HPMC (0.3%) | | | | |
| 8ARM-20k-NH2 | 0.10M phosphate, | 4.8 | 90 | N/A |
| 4ARM-20k-SGA | pH 7.80 | | | |
| 1.5% HPMC | | | | |
| 8ARM-20k-AA/8ARM-20k-NH2 | 0.10M phosphate, | 4.8 | 90 | 16 |
| (75/25) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 105 | 21 |
| (70/30) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 120 | N/A |
| (50/50) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 70 | 7 |
| (70/30) | pH 7.46 | | | |
| 8ARM-15k-SG | | | | |
| HPMC (0.3%) | | | | |
| 4ARM-20k-AA/8ARM-20k-NH2 | 0.10M phosphate, | 4.8 | 260 | 10 |
| (70/30) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 70 | 17 |
| (60/40) | pH 7.46 | | | |
| 8ARM-15k-SG | | | | |
| HPMC (0.3%) | | | | |
| 8ARM-20k-AA | 0.10M phosphate, | 4.8 | 85 | 7 |
| 4ARM-20k-SGA | pH 7.46 | | | |
| HPMC (0.3%) | | | | |
| 8ARM-20k-AA/8ARM-20k-NH2 | 0.10M phosphate, | 4.8 | 95 | 13 |
| (70/30) | pH 7.46 | | | |
| 4ARM-20k-SGA | | | | |
| HPMC (0.3%) | | | | |
| 8ARM-20k-AA/8ARM-20k-NH2 | 0.10M phosphate, | 4.8 | 95 | 10 |
| (75/25) | pH 7.46 | | | |
| 4ARM-20k-SGA | | | | |
| HPMC (0.3%) | | | | |
| 8ARM-20k-AA/8ARM-20k-NH2 | 0.10M phosphate, | 4 | 110 | In Progress |
| (75/25) | pH 7.58 | | | |
| 4ARM-20k-SGA | | | | |
| HPMC (0.3%) | | | | |
| 8ARM-20k-AA/8ARM-20k-NH2 | 0.10M phosphate, | 3.5 | 150 | In Progress |
| (75/25) | pH 7.58 | | | |
| 4ARM-20k-SGA | | | | |
| HPMC (0.3%) | | | | |
| 8ARM-20k-AA/8ARM-20k-NH2 | 0.10M phosphate, | 3 | 190 | In Progress |
| (75/25) | pH 7.58 | | | |
| 4ARM-20k-SGA | | | | |
| HPMC (0.3%) | | | | |

(B)

| | | | | | | | Polymer % |
|---|---|---|---|---|---|---|---|
| Pre-formulation | | | Wt | | | Arms | Solution |
| Components | MW | Mmoles | (g) | Arm | mmoles | Eq | (w/v) |
| 8ARM-20k-NH2 | 20000 | 1000 | 0.04 | 8 | 0.002 | 0.016 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 8ARM-20k-NH2 | 20000 | 1000 | 0.08 | 8 | 0.004 | 0.032 | |
| 8ARM-15k-SG | 15000 | 1000 | 0.06 | 8 | 0.004 | 0.032 | |
| Buffer Volume (phosphate) | | | 2.9 | | | | 4.8 |
| Viscosity Enhancer | 0.3% HPMC | | | | | | |
| 8ARM-20k-AA | 20000 | 1000 | 0.04 | 8 | 0.002 | 0.016 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 4ARM-20k-AA | 20000 | 1000 | 0.06 | 4 | 0.003 | 0.012 | |
| 8ARM-20k-NH2 | 20000 | 1000 | 0.02 | 8 | 0.001 | 0.008 | |
| 4ARM-20k-SGA | 20000 | 1000 | 0.1 | 4 | 0.005 | 0.02 | |
| Buffer Volume (phosphate) | | | 3.6 | | | | 5.0 |
| Viscosity Enhancer | 0.3% HPMC | | | | | | |
| 4ARM-20k-AA | 20000 | 1000 | 0.12 | 4 | 0.006 | 0.024 | |
| 8ARM-20k-NH2 | 20000 | 1000 | 0.04 | 8 | 0.002 | 0.016 | |
| 8ARM-15k-SG | 15000 | 1000 | 0.075 | 4 | 0.005 | 0.02 | |
| Buffer Volume (phosphate) | | | 4.9 | | | | 4.8 |
| Viscosity Enhancer | 0.3% HPMC | | | | | | |
| 8ARM-20k-AA | 20000 | 1000 | 0.06 | 8 | 0.003 | 0.024 | |
| 8ARM-20k-NH2 | 20000 | 1000 | 0.02 | 8 | 0.001 | 0.008 | |
| 4ARM-20k-SGA | 20000 | 1000 | 0.16 | 4 | 0.008 | 0.032 | |
| Buffer Volume (phosphate) | | | 5 | | | | 4.8 |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.02 | 8 | 0.001 | 0.008 | |
| 4ARM-20k-SGA | 20000 | 1000 | 0.1 | 4 | 0.005 | 0.02 | |
| Buffer Volume (phosphate) | | | 3.1 | | | | 4.8 |
| Viscosity Enhancer | 0.3% HPMC | | | | | | |
| 8ARM-20k-AA | 20000 | 1000 | 0.02 | 8 | 0.001 | 0.008 | |
| 8ARM-20k-NH2 | 20000 | 1000 | 0.02 | 8 | 0.001 | 0.008 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 8ARM-20k-AA | 20000 | 1000 | 0.035 | 8 | 0.00175 | 0.014 | |
| 8ARM-20k-NH2 | 20000 | 1000 | 0.015 | 8 | 0.00075 | 0.006 | |
| 4ARM-20k-SGA | 20000 | 1000 | 0.1 | 4 | 0.005 | 0.02 | |
| Buffer Volume (phosphate) | | | 3.1 | | | | 4.8 |
| Viscosity Enhancer | 0.3% HPMC | | | | | | |
| 8ARM-20k-AA | 20000 | 1000 | 0.039 | 8 | 0.00195 | 0.0156 | |
| 8ARM-20k-NH2 | 20000 | 1000 | 0.021 | 8 | 0.00105 | 0.0084 | |
| 4ARM-20k-SGA | 20000 | 1000 | 0.12 | 4 | 0.006 | 0.024 | |
| Buffer Volume (phosphate) | | | 3.75 | | | | 4.8 |
| Viscosity Enhancer | 0.3% HPMC | | | | | | |
| 8ARM-20k-AA | 20000 | 1000 | 0.09 | 8 | 0.0045 | 0.036 | |
| 8ARM-20k-NH2 | 20000 | 1000 | 0.03 | 8 | 0.0015 | 0.012 | |
| 4ARM-20k-SGA | 20000 | 1000 | 0.24 | 4 | 0.012 | 0.048 | |
| Buffer Volume (phosphate) | | | 9 | | | | 4.0 |
| Viscosity Enhancer | 0.3% HPMC | | | | | | |
| 8ARM-20k-AA | 20000 | 1000 | 0.075 | 8 | 0.00375 | 0.03 | |
| 8ARM-20k-NH2 | 20000 | 1000 | 0.025 | 8 | 0.00125 | 0.01 | |
| 4ARM-20k-SGA | 20000 | 1000 | 0.2 | 4 | 0.01 | 0.04 | |
| Buffer Volume (phosphate) | | | 8.55 | | | | 3.5 |
| Viscosity Enhancer | 0.3% HPMC | | | | | | |
| 8ARM-20k-AA | 20000 | 1000 | 0.06 | 8 | 0.003 | 0.024 | |
| 8ARM-20k-NH2 | 20000 | 1000 | 0.02 | 8 | 0.001 | 0.008 | |
| 4ARM-20k-SGA | 20000 | 1000 | 0.16 | 4 | 0.008 | 0.032 | |
| Buffer Volume (phosphate) | | | 8 | | | | 3.0 |

| | |
|---|---|
| Viscosity Enhancer | 0.3% HPMC |

TABLE 8
(A) Summary of the reaction details for several representative sticky formulations; (B)
more detailed tabulation of a selection of the reaction details including moles
(degradation times were measured in phosphate buffered saline (PBS) at 37° C.).
(A)

| | | | | | Estim. | Appr. |
|---|---|---|---|---|---|---|
| Components (Arm | | Poly. | Buffer Type & | | Deg. | Gel |
| Equiv. Mol %) | | Conc. | Components | | Time | Time |
| 4ARM-20k-SGA | 100% | 5% | Liquid | 0.10M | 2 to 4 weeks | 125 s |
| 8ARM-20k-AA | 65% | | 2.5 mL | Phosphate, | | |
| 8ARM-20k-NH2 | 35% | | | pH 7.58 | | |
| HPMC | 0.3% | | | | | |
| 4ARM-20k-SGA | 100% | 5% | Liquid | 0.10M | 2 weeks | 115 s |
| 8-ARM-20k-AA | 75% | | 2.5 mL | Phosphate, | | |
| 8ARM-20k-NH2 | 25% | | | pH 7.58 | | |
| HPMC | 0.3% | | | | | |
| 4ARM-20k-SGA | 100% | 5% | Liquid | 0.10M | 2 weeks | 155 s |
| 8ARM-20k-AA | 70% | | 2.5 mL | Phosphate, | | |
| 8ARM-20k-NH2 | 30% | | | pH 7.58 | | |
| HPMC | 0.3% | | | | | |
| 4ARM-20k-SGA | 100% | 5% | Liquid | 0.10M | 2 weeks | 110 s |
| 8ARM-20k-AA | 75% | | 2.5 mL | Phosphate, | | to |
| 8ARM-20k-NH2 | 25% | | | pH 7.58 | | 125 s |
| HPMC | 0.3% | | | | | |
| 4ARM-20k-SGA | 100% | 5% | Liquid | 0.10M | 2 weeks | 122 s |
| 8ARM-20k-AA | 75% | | 2.5 mL | Phosphate, | | |
| 8ARM-20k-NH2 | 25% | | | pH 7.58 | | |
| HPMC | 0.3% | | | | | |
| 4ARM-20k-SGA | 100% | 5% | Liquid | 0.10M | 2 weeks | 90 s |
| 8ARM-20k-AA | 75% | | 2.5 mL | Phosphate, | | to |
| 8ARM-20k-NH2 | 25% | | | pH 7.58 | | 120 s |
| HPMC | 0.3% | | 1000 ppm | | | |
| | | | Denatonium | | | |
| | | | benzoate | | | |
| 4ARM-20k-SGA | 100% | 5% | Liquid | 0.10M | 2 weeks | 90 s |
| 8ARM-20k-AA | 75% | | 2.5 mL | Phosphate, | | to |
| 8ARM-20k-NH2 | 25% | | | pH 7.58 | | 120 s |
| HPMC | 0.3% | | 500 ppm | | | |
| | | | Denatonium | | | |
| | | | benzoate | | | |
| 4ARM-20k-SGA | 100% | 5% | Liquid | 0.10M | 2 weeks | 90 s |
| 8ARM-20k-AA | 75% | | 2.5 mL | Phosphate, | | to |
| 8ARM-20k-NH2 | 25% | | | pH 7.58 | | 120 s |
| HPMC | 0.3% | | 100 ppm | | | |
| | | | Denatonium | | | |
| | | | benzoate | | | |
| 4ARM-20k-SGA | 100% | 5% | Liquid | 0.10M | 2 weeks | 130 s |
| 8ARM-20k-AA | 70% | | 2.5 mL | Phosphate, | | |
| 8ARM-20k-NH2 | 30% | | | pH 7.58 | | |
| HPMC | 0.3% | | | | | |
| 4ARM-20k-SGA | 100% | 4% | Liquid | 0.10M | 2 weeks | 205 s |
| 8ARM-20k-AA | 60% | | 2.25 mL | Phosphate, | | to |
| 8-ARM-20k-NH2 | 40% | | | pH 7.46 | | 230 s |
| HPMC | 0.3% | | | | | |
| 4ARM-20k-SGA | 100% | 6% | Solid | 0.10M | 30-60 days | 90 s |
| 8ARM-20k-AA | 65% | | Freeze-dried | Phosphate, | | |
| | | | (Aldrich) | pH 7.4 | | |
| 8ARM-20k-NH2 | 35% | | Suggested use | | | |
| | | | w/2 mL | | | |
| | | | drug solution | | | |
| 4ARM-20k-SGA | 100% | 5% | Liquid | 0.10M | 2 weeks | 90 s |
| 8ARM-20k-AA | 75% | | 2.5 mL | Phosphate, | | to |
| 8ARM-20k-NH2 | 25% | | | pH 7.58 | | 120 s |
| HPMC | 0.3% | | 10000 ppm | | | |
| | | | Denatonium | | | |
| | | | benzoate | | | |
| 4ARM-20k-SGA | 100% | 5% | Liquid | 0.10M | 2 weeks | 115 s |
| 8ARM-20k-AA | 75% | | 2.5 mL | Phosphate, | | |
| 8ARM-20k-NH2 | 25% | | | pH 7.58 | | |
| HPMC | 0.3% | | | | | |
| 4ARM-20k-SGA | 100% | 5% | Liquid | 0.10M | 2 weeks | 150 s |
| 8ARM-20k-AA | 75% | | 2.5 mL | Phosphate, | | |
| 8ARM-20k-NH2 | 25% | | Using freeze- | pH 7.4 | | |
| | | | dried phosphate | | | |
| | | | 1% Denatonium | | | |
| | | | benzoate, 2% | | | |
| | | | Chlorhexidine | | | |
| 4ARM-20k-SGA | 100% | 6% | Solid | 0.10M | 2 weeks | 110 s |
| 8ARM-20k-AA | 75% | | Freeze-dried | Phosphate, | | |
| | | | (Aldrich) | pH 7.4 | | |
| 8ARM-20k-NH2 | 25% | | Suggested use | | | |
| | | | w/2 mL | | | |
| | | | drug solution | | | |
| 4ARM-20k-SGA | 100% | 6% | Liquid | 0.01M | 2 weeks | 27 min |
| 8ARM-20k-AA | 70% | | 2.0 mL | Phosphate, | | to |
| 8ARM-20k-NH2 | 30% | | Phosphate | 0.137M | | 31 min |
| HPMC | 0.3% | | Buffered | NaCl, | | |
| | | | Saline | 0.0027M | | |
| | | | (PBS) | KCl, | | |
| | | | | pH 7.2 | | |
| 4ARM-20k-SGA | 100% | 5% | Liquid | 0.10M | 2 weeks | 158 s |
| 8ARM-20k-AA | 70% | | 2.5 mL | Phosphate, | | |
| 8ARM-20k-NH2 | 30% | | Nolvasan (2% | pH 7.4 | | |
| | | | Chlorhexidine) | | | |

(B)

| | | | | | | | Pol. % |
|---|---|---|---|---|---|---|---|
| | | | Wt | | | Arms | Sol. |
| Components | MW | Mmoles | (g) | Arm | mmoles | Eq | (w/v) |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Buffer Volume (phosphate) | | | 2.5 | | | | 4.8 |
| Denatonium benzoate | | 1000 ppm | | | | | |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Buffer Volume (phosphate) | | | 2.5 | | | | 4.8 |
| Denatonium benzoate | | 500 ppm | | | | | |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Buffer Volume (phosphate) | | | 2.5 | | | | 4.8 |
| Denatonium benzoate | | 100 ppm | | | | | |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Buffer Volume (phosphate) | | | 2.5 | | | | 4.8 |
| Denatonium benzoate | | 10000 ppm | | | | | |
| Viscosity Enhancer | 0.3% HPMC | | | | | | |
| 8ARM-20k-AA | 20000 | 1000 | 0.03 | 8 | 0.0015 | 0.012 | |
| 8ARM-20k-NH2 | 20000 | 1000 | 0.01 | 8 | 0.0005 | 0.004 | |
| 4ARM-20k-SGA | 20000 | 1000 | 0.08 | 4 | 0.004 | 0.016 | |
| Solid Phosphate | | | 0.043 | | | | |
| Nolvasan Volume (2% | | | 2.5 | | | | 4.8 |
| chlorhexidine) | | | | | | | |
| Denatonium benzoate | | 10000 ppm | | | | | |
| 8ARM-20k-AA | 20000 | 1000 | 0.026 | 8 | 0.0013 | 0.0104 | |
| 8ARM-20k-NH2 | 20000 | 1000 | 0.014 | 8 | 0.0007 | 0.0056 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 8ARM-20k-AA | 20000 | 1000 | 0.028 | 8 | 0.0014 | 0.0112 | |
| 8ARM-20k-NH2 | 20000 | 1000 | 0.012 | 8 | 0.0006 | 0.0048 | |
| 4ARM-20k-SGA | 20000 | 1000 | 0.08 | 4 | 0.004 | 0.016 | |
| Buffer Volume (phosphate) | | | 2.5 | | | | 4.8 |
| Viscosity Enhancer | 0.3% HPMC | | | | | | |
| 8ARM-20k-AA | 20000 | 1000 | 0.018 | 8 | 0.0009 | 0.0072 | |
| 8ARM-20k-NH2 | 20000 | 1000 | 0.012 | 8 | 0.0006 | 0.0048 | |
| 4ARM-20k-SGA | 20000 | 1000 | 0.06 | 4 | 0.003 | 0.012 | |
| Buffer Volume (phosphate) | | | 2.25 | | | | 4 |
| Viscosity Enhancer | 0.3% HPMC | | | | | | |
| 8ARM-20k-AA | 20000 | 1000 | 0.026 | 8 | 0.0013 | 0.0104 | |
| 8ARM-20k-NH2 | 20000 | 1000 | 0.014 | 8 | 0.0007 | 0.0056 | |
| 4ARM-20k-SGA | 20000 | 1000 | 0.08 | 4 | 0.004 | 0.016 | |
| Solid Phosphate | | | 0.035 | | | | 6 |
| Drug Solution | 2.0 mL | | | | | | |
| 8ARM-20k-AA | 20000 | 1000 | 0.027 | 8 | 0.00135 | 0.0108 | |
| 8ARM-20k-NH2 | 20000 | 1000 | 0.009 | 8 | 0.00045 | 0.0036 | |
| 4ARM-20k-SGA | 20000 | 1000 | 0.072 | 4 | 0.0036 | 0.0144 | |
| Solid Phosphate | | | 0.035 | | | | 5.4 |
| Drug Solution | 2.0 mL | | | | | | |
| 8ARM-20k-AA | 20000 | 1000 | 0.028 | 8 | 0.0014 | 0.0112 | |
| 8ARM-20k-NH2 | 20000 | 1000 | 0.012 | 8 | 0.0006 | 0.0048 | |
| 4ARM-20k-SGA | 20000 | 1000 | 0.08 | 4 | 0.004 | 0.016 | |
| Buffer Volume (phosphate) | | | 2 | | | | 6 |
| Viscosity Enhancer | 0.3% HPMC | | | | | | |
| 8ARM-20k-AA | 20000 | 1000 | 0.028 | 8 | 0.0014 | 0.0112 | |
| 8ARM-20k-NH2 | 20000 | 1000 | 0.012 | 8 | 0.0006 | 0.0048 | |
| 4ARM-20k-SGA | 20000 | 1000 | 0.08 | 4 | 0.004 | 0.016 | |
| Solid Phosphate | | | 0.043 | | | | |
| Nolvasan Volume (2% | | | 2.5 | | | | 4.8 |
| chlorhexidine) | | | | | | | |

| | |
|---|---|
| Denatonium benzoate | 1% |

### Cytotoxicity & Hemolysis Evaluation

Several polymer samples were sent out to NAMSA for cytotoxicity and hemolysis evaluation. Cytotoxic effects were evaluated according to ISO 10993-5 guidelines. Hemolysis was evaluated according to procedures based on ASTM F756 and ISO 10993-4.

The polymer 8ARM-20k-NH2 & 4ARM-20k-SGA at 4.8% solution with 0.3% HPMC was found to be non-cytotoxic and non-hemolytic. The polymer 8ARM-20k-AA/8ARM-20k-NH2 (70/30) & 4ARM-20k-SGA at 4.8% solution with 0.3% HPMC was found to be non-cytotoxic and non-hemolytic. In addition, formulations involving 4ARM-20kAA and 8ARM-15k-SG were also non-cytotoxic and non-hemolytic.

### Gel and Degradation Time Measurements

The gel time for all cases was measured starting from the addition of the ester until the gelation of the solution. The gel point was noted by pipetting 1 mL of the reaction mixture and observing the dropwise increase in viscosity until the mixture ceased to flow. Degradation of the polymers was performed by the addition of 1 to 10 mL of phosphate buffered saline per 1 g of the material in a 50 mL centrifuge tube and incubating the mixture at 37° C. A digital water bath was used to maintain the temperature. The degradation time was measured starting from the day of addition of the phosphate buffer to complete dissolution of the polymer into solution.

The effect of reaction buffer pH, phosphate concentration, polymer concentration and reaction temperature on the gel times were characterized. The buffer pH was varied from 7.2 to 8.0 by the dropwise addition of either 50% aqueous NaOH or 6.0 N HCl. Phosphate concentrations of 0.01, 0.02 and 0.05 M were prepared and adjusted to pH 7.4. Polymer concentrations from 2 to 20% solution were studied. Reaction temperatures of 5, 20, and 37° C. were tested by keeping the monomers, buffers, and reaction mixture at the appropriate temperature. The 5° C. environment was provided by a refrigerator and the 37° C. temperature was maintained via the water bath. Room temperature was found to be 20° C.

The effect of degradation buffer pH and the proportion of degradable amine in the polymer formulation on the degradation times were explored. The degradation buffer pH was varied from 7.2 to 9.0 by the dropwise addition of either 50% aqueous NaOH or 6.0 N HCl. The degradable amine components studied were either the 4ARM-20k-AA or the 8ARM-20k-AA, and the percent of degradable amine relative to the non-degradable amine was varied from 50 to 100%.

The degradation time is largely dependent on the buffer pH, temperature, and the biocompatible pre-formulation monomers used. Degradation occurs primarily through ester bond hydrolysis; in biological systems, enzymatic pathways may also play a role. Error! Reference source not found. compares the degradation times of formulations with 4ARM-20k-AA and 8ARM-20k-AA in varying amounts. In general, increasing the amount of degradable acetate amine in relation to the non-degradable amine decreases the degradation times. Additionally, in some instances, the 8ARM-20k-AA exhibits a longer degradation time than the 4ARM-20k-AA per mole equivalent, which becomes especially apparent when the percent of acetate amine drops below 70%.

The effect of the buffer pH on the degradation time was investigated. The pH range between 7.2 and 9.0 was studied. In general, a high pH environment results in a greatly accelerated degradation. For example, an increase in pH from approximately 7.4 to 7.7 decreases the degradation time by about half.

The degradation time of different Acetate Amine formulations was evaluated. The formulation with 70% Acetate Amine has a degradation time of approximately 14 days whereas the formulation with 62.5% Acetate Amine has a degradation time of approximately 180 days.

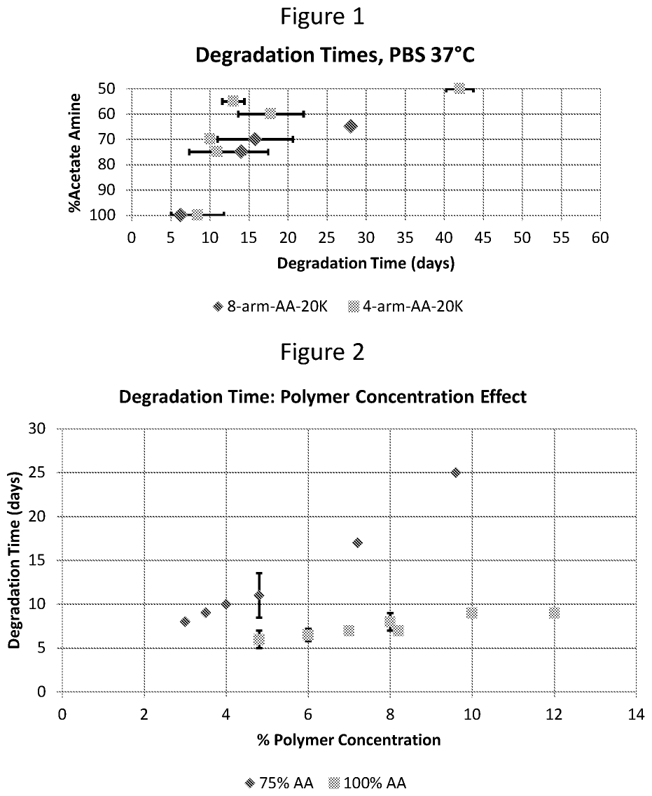

FIG. 2 shows the effect of polymer concentration on degradation time for different Acetate Amine formulations, where increasing polymer concentration slightly increases the degradation time (75% Acetate Amine formulation). This effect is less apparent for 100% Acetate Amine formulation, where the rate of ester hydrolysis is more significant.

The monomers used in the formulations have also been found to play a role in the way the polymer degrades. For the 8ARM-20k-AA/8ARM-20k-NH2 (70/30) & 4ARM-20k-SGA polymer, degradation occurred homogeneously throughout the material, resulting in a “smooth” degradation process. The polymer absorbed water and swelled slightly over the initial few days. Then, the polymer became gradually softer yet maintained its shape. Finally, the polymer lost its shape and became a highly viscous fluid.

Fragmenting degradation processes are observed when the amount of degradable amine becomes low, non-degradable regions in the polymer may occur. For instance a 4ARM-20k-AA/8ARM-20k-NH2 (70/30) & 4ARM-20k-SGA formulation degraded into several large fragments. For applications where the polymers are subjected to great forces, fragmentation may also occur as the polymer becomes softer and weaker over time.

Polymer Concentration

More dilute polymer solutions may be employed with minimal changes in the mechanical properties. For the formulation 8ARM-20k-AA-20K/8ARM-20k-NH2 (75/25) with 4ARM-20k-SGA and 0.3% HPMC, polymer concentrations of 3.0, 3.5 and 4.0% were studied. The gel times increased steadily as the polymer concentration was lowered. The firmness decreased slightly as the polymer concentration was lowered. There was essentially no change in the polymer adhesive properties. The elastic modulus decreased slightly as the polymer concentration was lowered.

TABLE 9
(A) Reaction details for specific sticky formulation; (B) Formulation results for a specific
sticky formulation with a variety of viscosity enhancing agents (the biocompatible hydrogel surface
spread test is conducted on a hydrophilic biocompatible hydrogel surface composed of 97.5% water
at an angle of approximately 30°; one drop of the polymer solution from a 22 gauge needle is
applied to the surface before gelation); (C) the clarity of solutions containing a variety of viscosity
enhancing agents, as measured by the % transmission at 650 nm.
(A)

| Pre-formulation | | | | | | |
|---|---|---|---|---|---|---|
| Components | MW | wt (g) | Arm | mmoles | Arms Eq | % Solution |
| 8ARM-20k-NH2 | 20000 | 0.04 | 8 | 0.002 | 0.016 | |
| 4ARM-20k-SGA | 20000 | 0.08 | 4 | 0.004 | 0.016 | |

| | | |
|---|---|---|
| Phosphate buffer | 2.5 mL 0.10M, pH 7.80 | 4.8 |

(B)

| Viscous | Approx. | Gel | Hydrogel Surface | |
|---|---|---|---|---|
| Agent | Viscosity | Time | Spread Test | |
| % (w/w) | (cP) | (s) | Category | Notes |
| 0 (Original | 1.1 | 80 | 2 | Rigid, has “bounce”. Slight elasticity. |
| Formulation) | | | | |
| 5% PVP | 1 to 5 | 90 | 2 to 3 | No change, except fora slight increase |
| | | | | in elasticity. |
| 10% PVP | 3 to 5 | 90 | 2 to 3 | Slightly opaque, moderate increase in |
| | | | | elasticity. Slippery. |
| 15% PVP | 5 to 10 | 100 | 2 to 3 | Opaque, definite increase in elasticity. |
| | | | | Slippery when wet, slightly sticky |
| | | | | when dry. |
| 20% PVP | 10 | 110 | 2 | Opaque, definite increase in elasticity. |
| | | | | Slippery when wet, very sticky when |
| | | | | dry. |
| 0.3% HPMC | 8.4 | 80 | 2 | No change. |
| 1.0% HPMC | 340.6 | 90 | 1 | No change. |
| 1.25% HPMC | 1,000 | 90 | 1 | No change. |
| 1.5% HPMC | 2,000 | 100 | 1 | Slightly softer, lacks “bounce”. |
| 2.0% HPMC | 4,000 | 100 | 1 | Slightly softer, lacks “bounce”. |
| | | | | Slippery. |

Hydrogel Surface Spread Test Categories: 1) No spreading, tight drops that
stay in place; 2) Mild spreading, drops drip slowly down;
3) Severe spreading, drops completely wet surface. Water is in category 3.
(C)

| Sample | % Transmission @ 650 nm |
|---|---|
| 0.10M phosphate buffer, pH 7.80 | 100.0% |
| 10% PVP | 99.9% |
| 1.5% HPMC | 95.7% |
| 1.0% HPMC | 96.8% |
| 0.5% HPMC | 99.1% |
| 0.1% HPMC | 99.6% |

Methylcellulose (MC) was found to behave similarly to hypromellose (HPMC) and provided workable viscous solutions in the concentration range of 0 to 2% (w/w). However, the HPMC dissolved more readily than the MC, and the HPMC solutions possessed greater optical clarity; thus the use of HPMC was favored. Povidone (PVP) dissolved easily in the buffer, but provided minimal viscosity enhancement even at 20% (w/w). Higher molecular weight grades of PVP are available, but have not yet been explored.

For the most part, the polymers remain unchanged by the addition of low concentrations of HPMC or PVP. However, there was a noticeable change in the polymer around 0.3% HPMC that was characterized by an enhanced elasticity, as evidenced by the ability of the material to elongate more than usual without breakage. Above 1.5% HPMC, the polymer became slightly softer and exhibited less bounce. The gel times also remained within 10 seconds of the gel time for the formulation with no viscous agent. In the case of PVP, significant changes in the polymer occurred above 10% PVP. The polymer became more opaque with a noticeable increase in elasticity and stickiness. At 15% to 20% PVP, the polymer became similar to the sticky materials, but with a better mechanical strength. The gel times also increased by roughly 20 seconds relative to the formulation with no viscous agent. Thus, the addition of lower concentrations of PVP or HPMC to the polymer solutions may be beneficial in improving the polymer's elasticity and lubricity.

The results of the biocompatible hydrogel surface spread test show that most formulations belong in category 2.

Based on these observations, a formulation utilizing 0.3% HPMC was chosen for further evaluation. Above 1.0% HPMC, the solutions became significantly more difficult to mix and dissolution of the monomers became an issue. At 0.5% HPMC and above, the formation of air bubbles during mixing became significant. Furthermore, the solutions were not easily filtered through a 0.5 μm syringe filter to remove the bubbles. However, the 0.3% HPMC solution was easily filtered even after moderate mixing, resulting in a bubble-free, optically clear polymer.

### Viscosity Measurements

The viscosities of the resulting buffer solutions were measured with the appropriately sized Cannon-Fenske viscometer tube from Ace Glass. Viscometer sizes used ranged from 25 to 300. Measurements of select solutions were performed in triplicate at both 20° C. and 37° C. The results are shown in Table 9B. To calculate the approximate dynamic viscosities, it was assumed that all the buffer solutions had the same density as water.

To characterize the rheology of the polymers during the gelation process, a size 300 viscometer was used with a formulation that was designed to gel after approximately 15 minutes. The formulation used involved the 8ARM-20k-NH2 with the 4ARM-20k-SGA ester at 2.5% solution and 0.3% HPMC. The reaction occurred in a 0.05 M phosphate buffer at a pH of 7.2. Thus, one viscosity measurement with the size 300 viscometer was obtained in about one minute and subsequent measurements may be obtained in quick succession up to the gel point.

### Hydrogel Surface Spread Test

To model the performance of the polymer solutions on a hydrophilic surface the extent of spreading and dripping of droplets on a high water content biocompatible hydrogel polymer matrix surface at an incline of about 30° was recorded. The biocompatible hydrogel polymer matrix was made by dissolving 0.10 g (0.04 mol arm eq.) of 8ARM-20k-NH2in 7 mL 0.05 M phosphate buffer at pH 7.4 in a Petri-dish, followed by the addition of 0.075 g (0.04 mol arm eq.) of 8ARM-15k-SG ester. The solution was stirred with a spatula for 10 to 20 seconds and allowed to gel, which typically took 5 to 10 minutes. The water content of the resulting polymer was 97.5%.

The test was performed by first preparing the polymer solution in the usual fashion. After thorough mixing, the polymer solution was dispensed dropwise through a 22 gauge needle onto the biocompatible biocompatible hydrogel polymer matrix surface. The results are shown in Table 9B and were divided into three general categories: 1) no spreading, tight drops that stay in place; 2) mild spreading, drops drip slowly down; 3) severe spreading, drops completely wet surface. Water is in category 3.

### Swelling & Drying Measurements

The extent of swelling in the polymers during the degradation process was quantified as the liquid uptake of the polymers. A known mass of the polymer was placed in PBS at 37° C. At specified time intervals, the polymer was isolated from the buffer solution, patted dry with paper towels and weighed. The percent increase in the mass was calculated from the initial mass.

The fate of the polymers in air under ambient conditions was quantified as the weight loss over time. A polymer film of about 1 cm thickness was placed on a surface at 20° C. Mass measurements were performed at set intervals. The percent weight loss was calculated from the initial mass value.

The percent of water uptake by the 8ARM-20k-NH2/4ARM-20k-SGA polymers with 0, 0.3 and 1.0% HPMC was investigated. The 1.0% HPMC polymer absorbed up to 30% of its weight in water until day 20. After day 20, the polymer returned to about 10% of its weight in water. In comparison, the 0% HPMC polymer initially absorbed up to 10% of its weight in water, but began to lose water gradually, hovering about 5% of its weight in water. The 0.3% HPMC polymer behaved in an intermediate fashion. It initially absorbed up to 20% of its weight in water, but returned to about 10% of its weight in water after a week and continued to slowly lose water.

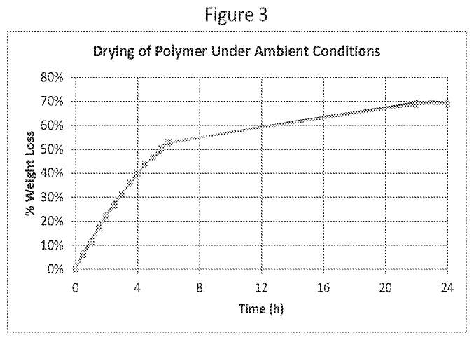

The percent of weight loss under ambient conditions over 24 hours by the 8ARM-20k-AA/8ARM-20k-NH2 (75/25) & 4ARM-20k-SGA polymer with 0.3% HPMC and 1.0% HPMC is shown in FIG. 3. Ambient conditions were roughly 20° C. and 30 to 50% relative humidity. The rate of water loss was fairly constant over 6 hours at about 10% per hour. After 6 hours, the rate slowed significantly as the polymer weight approached a constant value. The rate of water loss is expected to vary based on the polymer shape and thickness, as well as the temperature and humidity.

### Specific Gravity Measurements

The specific gravity of the polymers was obtained by preparing the polymer solution in the usual fashion and pipetting 1.00 mL of the thoroughly mixed solution onto an analytical balance. The measurements were performed in triplicate at 20° C. The specific gravity was calculated by using the density of water at 4° C. as the reference.

The specific gravity of the polymers did not differ significantly from that of the buffer solution only, both of which were essentially the same as the specific gravity of water. Exceptions may occur when the polymer solution is not filtered and air bubbles become embedded in the polymer matrix.

### Barium Sulfate Suspensions

For imaging purposes, barium sulfate was added to several polymer formulations as a radiocontrast agent. Barium sulfate concentrations of 1.0, 2.0, 5.0 and 10.0% (w/v) were explored. The viscosity of the resulting polymer solutions was measured and the effect of barium sulfate addition on the polymer gel times and syringability characteristics were also studied.

Barium sulfate concentrations of 1.0, 2.0, 5.0 and 10.0% (w/v) were explored. The opaque, milky white suspensions formed similarly opaque and white polymers. No changes in the gel times were observed. Qualitatively, the polymers appeared to have similar properties to that of polymers without barium sulfate. All formulations were able to be readily dispensed through a 22 gauge needle.

The viscosity measurements for barium sulfate concentrations of 1.0, 2.0, 5.0 and 10.0% was measured. The viscosity remained relatively stable up to 2.0%; at 5.0%, the viscosity increased slightly to about 2.5 cP. There was a sharp increase in the viscosity to nearly 10 cP as the concentration approached 10.0%. Thus, a barium sulfate concentration of 5.0% was chosen as a balance between high contrast strength and similarity to unmodified polymer formulations. Biocompatible Hydrogel Firmness, Elastic Modulus, and Adhesion

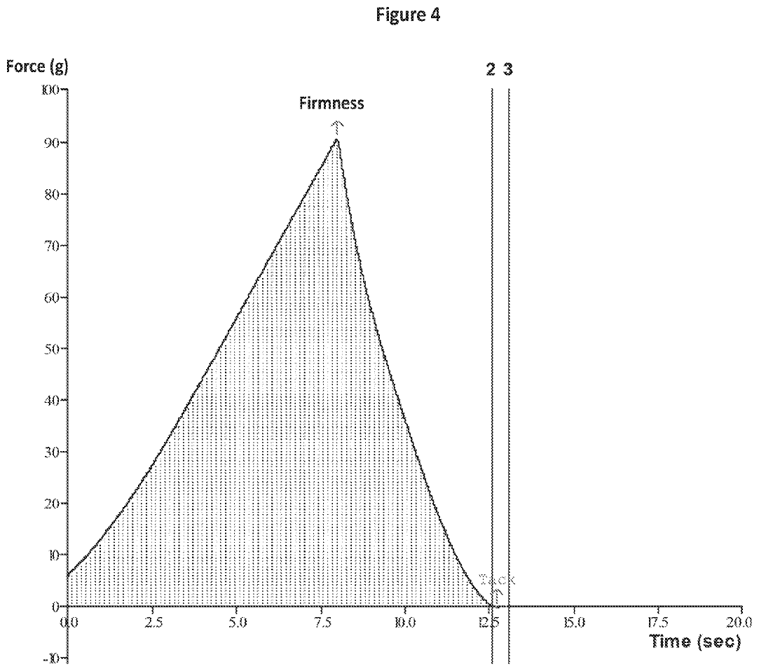

The firmness of the polymers was characterized by a Texture Analyzer model TA.XT.plus with Exponent software version 6.0.6.0. The method followed the industry standard “Bloom Test” for measuring the firmness of gelatins. In this test, the TA-8¼″ ball probe was used to penetrate the polymer sample to a defined depth and then return out of the sample to the original position. The peak force measured is defined as the “firmness” of the sample. For the polymers studied, a test speed of 0.50 mm/sec, a penetration depth of 4 mm, and a trigger force of 5.0 g were used. The polymers were prepared on a 2.5 mL scale directly in a 5 mL size vial to ensure consistent sample dimensions. The vials used were ThermoScientific/Nalgene LDPE sample vials, product #6250-0005 (LOT #7163281060). Measurements were conducted at 20° C. The polymers were allowed to rest at room temperature for approximately 1 hour before measuring. Measurements were performed in triplicate for at least three samples. A sample plot generated by the Exponent software running the firmness test is given in FIG. 4. The peak of the plot represents the point at which the target penetration depth of 4 mm was reached.

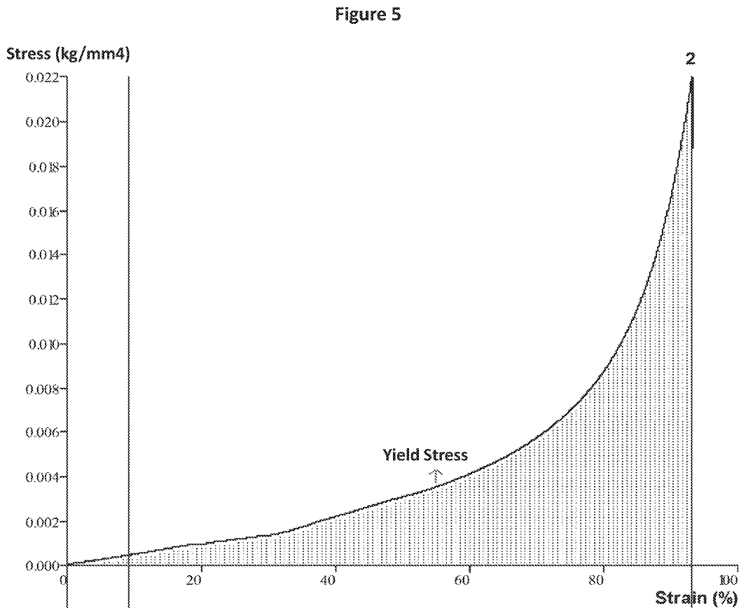

The elastic modulus of the polymers was characterized by a Texture Analyzer model TA.XT.plus with Exponent software version 6.0.6.0. In this test, the TA-19 Kobe probe was used to compress a polymer cylinder of known dimensions until fracture of the polymer occurs. The probe has a defined surface area of 1 cm². The modulus was calculated as the initial slope up to 10% of the maximum compression stress. For the polymers studied, a test speed of 5.0 mm/min and a trigger force of 5.0 g were used. The sample height was auto-detected by the probe. The polymers were prepared on a 2.5 mL scale directly in a 5 mL size vial cap to ensure consistent sample dimensions. The vials used were ThermoScientific/Nalgene LDPE sample vials, product #6250-0005 (LOT #7163281060). Measurements were conducted at 20° C. The polymers were allowed to rest at room temperature for approximately 1 hour before measuring. Measurements were performed for at least three samples. A sample plot generated by the Exponent software running the modulus test is given in FIG. 5. The polymers typically behaved elastically for the initial compression, as evidenced by the nearly linear plot.

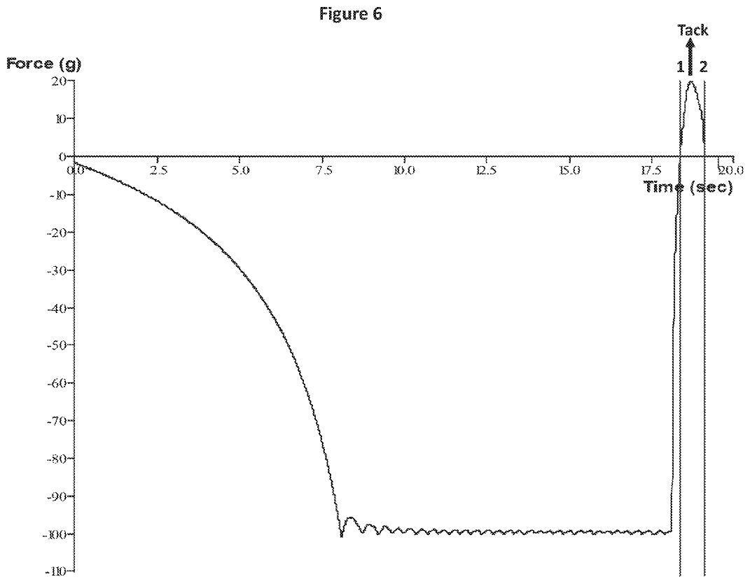

The adhesive properties of the polymers were characterized by a Texture Analyzer model TA.XT.plus with Exponent software version 6.0.6.0. In the adhesive test, the TA-57R 7 mm diameter punch probe was used to contact the polymer sample with a defined force for a certain amount of time, and then return out of the sample to the original position. An exemplary plot generated by the Exponent software running the adhesive test is given in FIG. 6. The plot begins when the probe hits the surface of the polymer. The target force is applied on the sample for a defined unit of time, represented by the constant force region in the plot. Then, the probe returns out of the sample to the original position and the adhesive force between the probe and the sample is measured as the “tack”, which is the peak force required to remove the probe from the sample. Other properties that were measured include the adhesion energy or the work of adhesion, and the material's “stringiness.” The adhesion energy is simply the area under the curve representing the tack force. Thus, a sample with a high tack and low adhesion energy will qualitatively feel very sticky, but may be cleanly removed with a quick pull; a sample with a high tack and high adhesion energy will also feel very sticky, but the removal of the material will be more difficult and may be accompanied by stretching of the polymer, fibril formation and adhesive residues. The elasticity of the polymer is proportional to the measured “stringiness”, which is the distance the polymer stretches while adhered to the probe before failure of the adhesive bond. For the polymers studied, a test speed of 0.50 mm/sec, a trigger force of 2.0 g, and a contact force of 100.0 g and contact time of 10.0 sec were used. The polymers were prepared on a 1.0 to 2.5 mL scale directly in a 5 mL size vial to ensure consistent sample surfaces. The vials used were Thermo Scientific/Nalgene LDPE sample vials. Measurements were conducted at 20° C. The polymers were allowed to rest at room temperature for approximately 1 hour before measuring. As reference materials, the adhesive properties of a standard Post-It Note® and Scotch Tape® were measured. All measurements were performed in triplicate. The averages and standard deviations were calculated.

The effect of HPMC addition to the mechanical properties of the polymers was explored, along with the effect of adding degradable 8ARM-20k-AA amine. Under the stated conditions of the firmness test, it was found that the addition of 0.3% HPMC decreased the firmness of the polymer by about half. This corresponds to a slight decrease in the elastic modulus. The 1.0% HPMC polymer had approximately the same firmness as the 0.3% HPMC polymer, but a slight decrease in the elastic modulus. The disparity between the firmness and modulus tests is likely due to experimental error. The polymer solutions were not filtered, so the presence of air bubbles likely increased the errors. The water content of the polymers may also change as the polymers were sitting in the air, essentially changing the physical properties of the materials.

It was found that the addition of the degradable 8ARM-20k-AA amine did not substantially change the measured values of the firmness or the elastic modulus. The measured values for a standard commercial Post-It™ Note are also included as a reference. The polymer tack was found to be around 40 mN, which is about three times less than that of a Post-It™ Note. The adhesive properties of the polymer were not found to vary with the addition of the degradable amine.

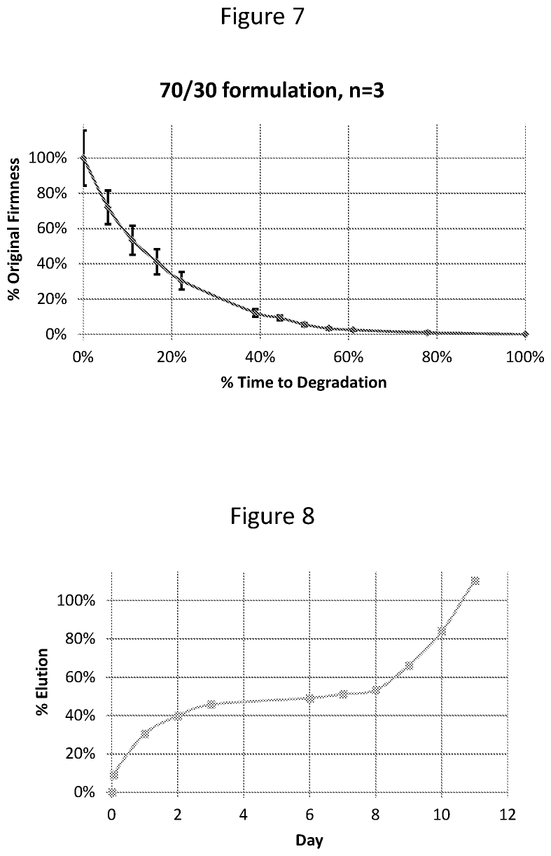

FIG. 7 shows the firmness vs. degradation time for the 8ARM-20k-AAI8ARM-20k-NH2 (70/30) & 4ARM-20k-SGA at 4.8% solution with 0.3% HPMC. The error bars represent the standard deviations of 3 samples. The degradation time for the polymer was 18 days. The firmness of the polymer strongly correlated with the extent of degradation. Swelling may also play a role during the early stages.

The effect of various additives to the formulation on the polymer properties was explored. Gel gel time, degradation time, firmness, adhesion and elastic modulus was measured for polymers prepared with varying combinations of 1% HPMC, 2% chlorhexidine and 1% denatonium benzoate. Essentially no change in the polymer properties were found except for formulations containing 2% chlorhexidine, which exhibited decreased firmness and elastic modulus. It was apparent from visual inspection of the polymer that the change was due to the detergent present in the Nolvasan solution used and not the chlorhexidine; the detergent caused heavy foaming during mixing that gelled into an aerated polymer.

### Optical Clarity

A Thermo Scientific GENESYS 10S UV-Vis spectrophotometer was used to measure the optical clarity of the viscous solutions. To a quartz cuvette, 1.5 mL of the sample solution was pipetted. The buffer solution with no additives was used as the reference. The stable % transmission of the sample was recorded at 650 nm.

To measure the light transmission of the polymers, 1 mL of polymer solution was filtered with a 5 μm filter into a cuvette before gelation. The cuvette was then placed horizontally so that the polymer gelled on the side of the cuvette as a film. The film thickness was found to be 3 mm. The polymer was allowed to cure for 15 minutes at room temperature before measuring the % light transmission at 400, 525 and 650 nm with air as the reference.

All of the viscous solutions under consideration were found to have acceptable to excellent optical clarity under the concentration ranges used (greater than 97% transmission). For the highly viscous solutions, air bubble formation during mixing was observed, which may be resolved by the addition of an anti-foaming agent, or through the use of a syringe filter (See Table 9C).

The polymers exhibited excellent optical clarities over the visible spectrum. The lowest % transmission relative to buffer only was 97.2% and the highest was 99.7%. The drop in the % transmission at lower wavelengths is likely due to some energy absorption as the ultraviolet region is approached.

### Drug Elution: General Procedures

A Thermo Scientific GENESYS 10S UV-Vis spectrophotometer was used to quantify the release of various drugs from several polymers. First, the reference drug or drug solution was dissolved in an appropriate solvent. Typically, phosphate buffered saline (PBS), ethanol or dimethylsulfoxide (DMSO) were used as the solvent. Next, the optimal absorption peak for identifying and quantifying the drug was determined by performing a scan of the drug solution between 200 and 1000 nm. With the absorption peak selected, a reference curve was established by measuring the peak absorbance for various concentrations of the drug. The different drug concentration solutions were prepared by standard dilution techniques using analytical pipettes. A linear fit of the absorbance vs. drug concentration resulted in a general equation that was used to convert the measured absorbance of the elution samples to the drug concentration.

The polymer was prepared with a known drug dosage in the same fashion as a doctor administering the polymer in a clinical setting. However, in this case the polymer was molded into a cylinder with a diameter of approximately 18 mm. The polymer cylinder was then placed in a 50 mL Falcon tube with a set amount of PBS and placed at 37° C. The temperature was maintained by a digitally controlled water bath.

Elution samples were collected daily by decanting the PBS solution from the polymer. The volume of sample collected was recorded. The polymer was placed in a volume of fresh PBS equivalent to the volume of sample that was collected and returned to 37° C. The elution sample was analyzed by first diluting the sample in the appropriate solvent using analytical pipettes such that the measured absorbance was in the range determined by the reference curve. The dilution factor was recorded. The drug concentration was calculated from the measured absorbance via the reference curve and the dilution factor. The drug amount was calculated by multiplying the drug concentration with the sample volume. The percent elution for that day was calculated by dividing the drug amount by the total amount of drug administered.

### Drug Elution: Chlorhexidine

The peak found between 255 and 260 nm was chosen and a reference curve was established by measuring the peak absorbance for 0, 0.5, 1, 2.5, 5, 10, 20, 40, and 50 ppm of chlorhexidine. Concentrations above 50 ppm did not exhibit linear behavior in peak absorbance.

The polymer was prepared with a commercial Nolvasan solution, which corresponds to a 2% chlorhexidine dose (50 mg). The elution volume was 2 mL of PBS per 1 g of polymer. The elution samples were stored at 20° C. The elution samples were analyzed by diluting the sample 1,000-fold with dimethyl sulfoxide (DMSO) in a quartz cuvette.

The chlorhexidine elution behavior proceeded similarly to previous experiments with other small molecules. Almost half of the chlorhexidine was released within the first three days. Then, the elution rate slowed dramatically for the next three to four days followed by another large release of chlorhexidine as the polymer degrades (FIG. 8).

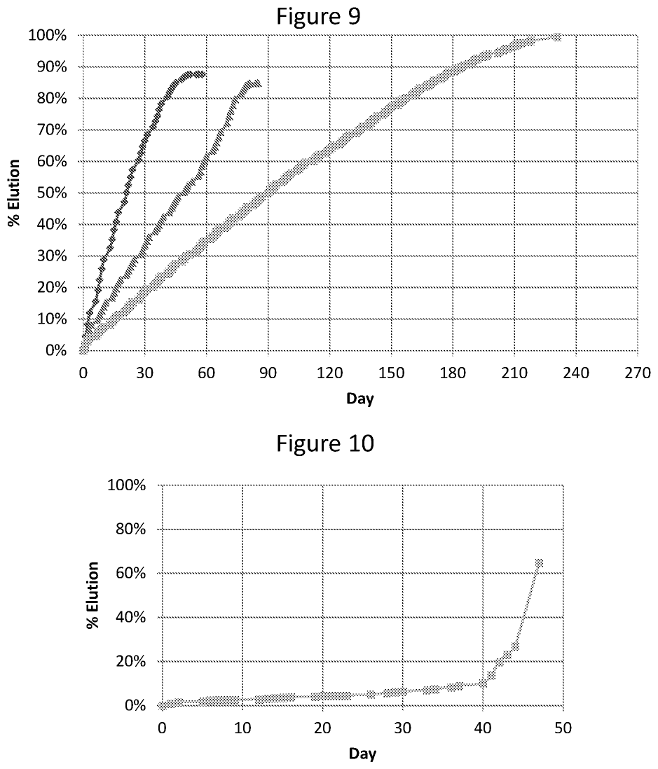

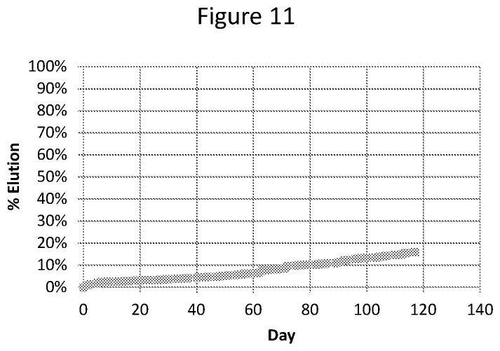

The elution of the steroidal drugs, triamcinolone and methylprednisolone, behaved similarly The first few days typically exhibit an elevated elution rate, presumably as weakly bound surface drug is released. Then, the elution is relatively constant at a rate that is related to the drug solubility. Finally, the remaining drug in the polymer is released as degradation begins. Several examples are given in FIG. 9, FIG. 10, and FIG. 11 of the control over the elution behavior that was developed. Drugs may be released over a short time (weeks) or long period (years, projected).

### Example 12: General Procedure for the Preparation of Polymerizable Biocompatible Pre-Formulations

Several representative formulations for both sticky and non-sticky films are listed in Table 10 along with specific reaction details. The films had thicknesses ranging from 100 to 500 μm, and may be layered with different formulations in a composite film.

TABLE 10
(A) Summary of the reaction details for several representative thin film
formulations; (B) more detailed tabulation of a selection of the reaction
details includingmoles (films ranged in thickness from 100 to 500 pm).
(A)

| | Amine/Ester | | % |
|---|---|---|---|
| Pre-formulation Components | Molar Ratio | Buffer | Solution |
| 4ARM-20k-AA & 8ARM-15k-SG | 1 | 0.15M | |
| | | phosphate, | 19.6 |
| | | pH 7.99 | |
| 4ARM-5k-NH2 & 4ARM-10k-SG | 4.5/1 | 0.05M | |
| | | phosphate, | 39 |
| | | pH 7.40 | |
| 4ARM-5k-NH2 & 4ARM-10k-SG | 1 | 0.05M | |
| | | phosphate, | 36.4 |
| | | pH 7.40 | |
| 4ARM-5k-NH2 & 4ARM-10k-SG & HPMC (1.25%) | 4.5/1 | 0.10M | |
| | | phosphate, | 39 |
| | | pH 7.80 | |
| 4ARM-2k-NH2 & 4ARM-10k-SG & HPMC (1.5%) | 8/1 | 0.10M | |
| | | phosphate, | 30.6 |
| | | pH 7.80 | |
| 4ARM-2k-NH2 & 4ARM-20k-SGA & MC (2%) | 8/1 | 0.15M | |
| | | phosphate, | 30 |
| | | pH 7.94 | |
| 4ARM-2k-NH2 & 4ARM-20k-SGA & MC (2%) | 10/1 | 0.15M | |
| | | phosphate, | 30 |
| | | pH 7.94 | |

(B)

| | | | | | | | Polymer |
|---|---|---|---|---|---|---|---|
| Pre-formulation | | | | | | Arms | % Solution |
| Components | MW | Mmoles | Wt (g) | Arm | mmoles | Eq | (w/v) |
| 4ARM-20k-AA | 20000 | 1000 | 0.2 | 4 | 0.01 | 0.04 | |
| 8ARM-15k-SG | 15000 | 1000 | 0.075 | 8 | 0.01 | 0.04 | |
| Buffer Volume (phosphate) | | | 1.4 | | | | 19.6 |
| 4ARM-5k-NH2 | 5000 | 1000 | 0.27 | 4 | 0.05 | 0.22 | |
| 4ARM-10k-SG | 10000 | 1000 | 0.12 | 4 | 0.01 | 0.05 | |
| Buffer Volume (phosphate) | | | 1 | | | | 39.0 |
| 4ARM-5k-NH2 | 5000 | 1000 | 0.17 | 4 | 0.03 | 0.14 | |
| 4ARM-10k-SG | 10000 | 1000 | 0.34 | 4 | 0.03 | 0.14 | |
| Buffer Volume (phosphate) | | | 1.4 | | | | 36.4 |
| 4ARM-5k-NH2 | 5000 | 1000 | 0.27 | 4 | 0.05 | 0.22 | |
| 4ARM-10k-SG | 10000 | 1000 | 0.12 | 4 | 0.01 | 0.05 | |
| Buffer Volume (phosphate) | | | 1 | | | | 39.0 |

| | |
|---|---|
| Viscosity Enhancer | 1.25% HPMC |

### Example 13: Preparation of Kits and their Use

Several kits were prepared with the polymer formulation tested earlier. The materials used to assemble the kits are listed in Table 11 and the formulations used are listed in Table 12. The kits are typically composed of two syringes, one syringe containing the solid components and the other syringe containing the liquid buffer. The syringes are connected via a mixing tube and a one-way valve. The contents of the syringes are mixed via opening the valve and transferring the contents of one syringe into the other, repeatedly, for 10 to 20 seconds. The spent syringe and mixing tube are then removed and discarded, and the active syringe is fitted with a dispensing unit, such as a needle or cannula, and the polymer solution is expelled until the onset of gelation. In other embodiments, the viscous solution impedes the dissolution of the solid components and thus a third syringe is employed. The third syringe contains a concentrated viscous buffer that enhances the viscosity of the solution once all the components have dissolved. In some embodiments, the optical clarity of the resulting polymer is improved through the addition of a syringe filter.

All of the formulations tested were easily dispensed through a 22 gauge needle. The mixing action between the two syringes was turbulent and the introduction of a significant amount of air bubbles was apparent. Gentle mixing results in a clear material free of bubbles. Alternatively, the use of a syringe filter was found to remove bubbles without any change in the polymer properties.

TABLE 11
Materials used to fabricate kits including vendor,
part number and lot number.

| Description | Vendor |
|---|---|
| Vincon Tubing, ⅛″ I.D. ¼″ | Ryan Herco Flow Solutions |
| O.D. 1/16″ wall, 100 Ft. | |
| 12 mL Luer-Lock Syringe | Tyco Healthcare, |
| | Kendall Monoject ™ |
| 3 mL Luer-Lock Syringe | Tyco Healthcare, |
| | Kendall Monoject ™ |
| One Way Stopcock, Female | QOSINA |
| Luer Lock to Male Luer | |
| Female Luer Lock Barb for ⅛″ | QOSINA |
| I.D. tubing, RSPC | |
| Non-vented Luer Dispensor Tip Cap, White | QOSINA |
| 32 mm Hydrophilic Syringe Filter, 5 micron | PALL ® Life Sciences |

TABLE 12
The detailed contents for four different kits; the solid components are in one syringe,
while the liquid components are in another syringe; a mixing tube connects the two syringes.

| Pre-formulation Components | MW | wt (g) | Arm | mmoles | Arms Eq | % Solution |
|---|---|---|---|---|---|---|
| 8ARM-20k-NH2 | 20000 | 0.04 | 8 | 0.002 | 0.016 | |
| 4ARM-20k-SGA | 20000 | 0.08 | 4 | 0.004 | 0.016 | |
| Phosphate buffer | | 2.5 mL 0.10M, pH 7.80 | | | | 4.8 |
| Viscosity Enhancer | | No viscosity enhancer | | | | |
| 8ARM-20k-NH2 | 20000 | 0.04 | 8 | 0.002 | 0.016 | |
| 4ARM-20k-SGA | 20000 | 0.08 | 4 | 0.004 | 0.016 | |
| Phosphate buffer | | 2.5 mL 0.10M, pH 7.80 | | | | 4.8 |
| Viscosity Enhancer | | 0.3% HPMC | | | | |
| 8ARM-20k-NH2 | 20000 | 0.04 | 8 | 0.002 | 0.016 | |
| 4ARM-20k-SGA | 20000 | 0.08 | 4 | 0.004 | 0.016 | |
| Phosphate buffer | | 2.5 mL 0.10M, pH 7.80 | | | | 4.8 |
| Viscosity Enhancer | | 7.5% Povidone | | | | |
| 8ARM-20k-NH2 | 20000 | 0.04 | 8 | 0.002 | 0.016 | |
| 4ARM-20k-SGA | 20000 | 0.08 | 4 | 0.004 | 0.016 | |

| | | |
|---|---|---|
| Phosphate buffer | 2.5 mL 0.10M, pH 7.80 | 4.8 |
| Viscosity Enhancer | 1.0% HPMC | |

Several additional kits were prepared with the polymer formulation that performed the best in initial trials. The materials used to assemble the kits are listed in Table 13. The kits are typically composed of two syringes, one syringe containing the solid components and the other syringe containing the liquid buffer. The syringes were loaded by removing the plungers, adding the components, purging the syringe with a gentle flow of nitrogen gas for 20 seconds, and then replacing the plunger. Finally, the plungers were depressed as much as possible to reduce the internal volume of the syringes. The specifications for the amounts of chemical components in the kits are listed in Table 14A. A summary describing the lots of kits prepared is listed in Table 14B.

The syringes were connected directly after uncapping, the male part locking into the female part. The contents of the syringes were mixed via transferring the contents of one syringe into the other, repeatedly, for 10 to 20 seconds. The spent syringe was then removed and discarded, and the active syringe was fitted with a dispensing unit, such as a needle or cannula, and the polymer solution was expelled until the onset of gelation. In other embodiments, the viscous solution impeded the dissolution of the solid components and thus a third syringe was employed. The third syringe contained a concentrated viscous buffer that enhanced the viscosity of the solution once all the components had dissolved.

All the formulations tested were easily dispensed through a 22 gauge needle. The mixing action between the two syringes was turbulent and the introduction of a significant amount of air bubbles was apparent. The use of a syringe filter was found to remove bubbles without any change in the polymer properties.

The prepared kits were placed into foil pouches along with one oxygen absorbing packet per pouch. The pouches were heat sealed with a CHTC-280 PROMAX tabletop chamber sealing unit. Two different modes of sealing were explored: under nitrogen and under vacuum. The settings for sealing under nitrogen were: 30 seconds of vacuum, 20 seconds of nitrogen, 1.5 seconds of heat sealing, and 3.0 seconds of cooling. The settings for sealing under vacuum were: 60 seconds of vacuum, 0 seconds of nitrogen, 1.5 seconds of heat sealing, and 3.0 seconds of cooling.

TABLE 13
Materials used to fabricate kits including vendor,
part number and lot number.

| Description | Vendor |
|---|---|
| 12 mL Male Luer-Lock Syringe | Tyco Healthcare, |
| | Kendall Monoject ™ |
| 5 mL Female Luer Lock Syringe, Purple | QOSINA |
| Male Luer Lock Cap, Non-vented | QOSINA |
| Female Non-vented Luer Dispensor Tip Cap, White | QOSINA |
| 100 cc oxygen absorbing packet | IMPAK |
| 6.25″ × 9″ OD PAKVF4 Mylar foil pouch | IMPAK |

TABLE 14
Specifications for kit components for the
8ARM-20k-AA/8ARM-20-NH2 & 4ARM-20k-
SGA formulation with 60, 65, 70 and 75% degradable
amine (A). LOT formulation summary (B).
(A)

| Pre- | | | | |
|---|---|---|---|---|
| formulation | Specifications | | | |
| Components | 60/40 | 65/35 | 70/30 | 75/25 |
| 8ARM- | 0.024-0.026 | 0.026-0.027 g | 0.028-0.029 g | 0.030-0.031 g |
| 20k-AA | g | | | |
| 8ARM- | 0.014-0.016 | 0.013-0.014 g | 0.011-0.012 g | 0.009-0.010 g |
| 20k-NH2 | g | | | |
| 4ARM- | 0.080-0.082 | 0.080-0.082 g | 0.080-0.082 g | 0.080-0.082 g |
| 20k-SGA | g | | | |

| | |
|---|---|
| Phosphate | 2.50 mL of 0.10M phosphate, |
| Buffer | pH 7.58, 0.30% HPMC (8.48 cSt +/− |
| | 0.06 @ 20° C.) |

(B)

| For- | | Sealing | |
|---|---|---|---|
| mulation | Buffer pH | Method | Notes |
| 60/40 | 7.46 | nitrogen | no nitrogen purging |
| 60/40 | 7.58 | nitrogen | of syringe |
| 60/40 | 7.72 | nitrogen | |
| 70/30 | 7.58 | vacuum | |
| 70/30 | 7.58 | vacuum | |
| 65/35 | 7.58 | vacuum | |
| 75/25 | 7.58 | vacuum | |
| 75/25 | 7.58 | vacuum | |
| 75/25 | 7.58 | nitrogen | |
| 65/35 | 7.58 | vacuum | |
| 65/35 | 7.58 | nitrogen | |

Several kits were prepared for use in beta testing. The materials used to assemble the kits are listed in Table 15. The kits are typically composed of two syringes, one syringe containing the solid components and the other syringe containing the liquid buffer. The syringes were loaded by removing the plungers, adding the components, purging the syringe with a gentle flow of inert gas for 10 seconds, and then replacing the plunger. Finally, the plungers were depressed as much as possible to reduce the internal volume of the syringes.

Alternatively, a single syringe kit may be prepared by loading the solid components into one female syringe along with a solid form of the phosphate buffer. The kit is then utilized in a similar fashion as the dual syringe kit, except the user may use a specified amount of a variety of liquids in a male syringe. Typically, any substance provided in a liquid solution for injection may be used. Some examples of suitable liquids are water, saline, Kenalog-10, Depo-Medrol and Nolvasan.

The kits are utilized in the following fashion. The syringes are connected directly after uncapping, the male part locking into the female part. The contents of the syringes are mixed via transferring the contents of one syringe into the other, repeatedly, for 10 to 20 seconds. The spent syringe is then removed and discarded, and the active syringe is fitted with a dispensing unit, such as a needle, a spray nozzle or a brush tip, and the polymer solution is expelled until the onset of gelation.

The prepared kits were placed into foil pouches along with one oxygen absorbing packet and one indicating silica gel packet per pouch. Labels were affixed to the pouches that displayed the product and company name, contact information, LOT and batch numbers, expiration date, and recommended storage conditions. A radiation sterilization indicator that changes color from yellow to red upon exposure to sterilizing radiation was also affixed to the upper left corner of the pouch. The pouches were heat sealed with a CHTC-280 PROMAX tabletop chamber sealing unit. The settings for sealing under vacuum were: 50 seconds of vacuum, 1.5 seconds of heat sealing, and 5.0 seconds of cooling.

An example detailing the lots of sterile kits prepared is listed in Table 15. A previous study found that if the loaded syringe was not purged with nitrogen before replacing the plunger during kit preparation, the sterile kits exhibited an increase in gel time of about 30 seconds relative to kits that had syringes flushed with nitrogen. No significant difference was found between kits that had been sealed under vacuum and kits that had been sealed under nitrogen. It was easily observable when the vacuum-sealed kits lost their seal, so it was decided to vacuum-seal all kits as standard procedure. The effects of including the oxygen absorbing packet and silica gel packet to the kits on the long term storage stability is currently under investigation.

TABLE 15
Materials used to fabricate kits including vendor, and part number.

| Description | Vendor | Part # |
|---|---|---|
| 10 mL Luer-Lok Syringe | BD | 309604 |
| Non-Vented Luer Dispenser | QOSINA | 65119 |
| Tip Cap, White | | |
| 5 mL Female Luer-Lock | QOSINA | C3610 |
| Syringe, Purple PP | | |
| Male Luer Lock Cap, Non-Vented, PP | QOSINA | 11166 |
| Brush tip | Flumatic | BT01225R |
| 5.25″ × 8″ PAKVF4D Mylar foil pouch | IMPAK | 0525MFDFZ08TE |
| 3.5″ × 6.5″ PAKVF4W Mylar foil pouch | IMPAK | 035MFW065Z |
| Radiation Sterilization Indicator | QOSINA | 13124 |
| 100 cc oxygen absorbing packet | IMPAK | OAP100 |
| Indicating silica gel | IMPAK | 401SG37 |

TABLE 16

| Example specifications for kit components for the | | | | |
|---|---|---|---|---|
| 8-arm-AA-20K/8-arm-NH2-20K & 4-arm-SGA-20K | | | | |
| formulation with 75% degradable amine (A). | | | | |
| LOT formulation summary (B). | | | | |
| (A) | | | | |
| Components | LOT# & Specifications | | | |
| 8ARM-20k-AA | 0.029-0.031 g | | | |
| 8ARM-20k-NH2 | 0.009-0.011 g | | | |
| 4ARM-20k-SGA | 0.079-0.081 g | | | |
| Phosphate Buffer | 2.50 mL of 0.10M | | | |
| | phosphate, pH 7.58, | | | |
| | 0.30% HPMC (8.48 | | | |
| | cSt +/− 0.06 @ 20° C.) | | | |
| LOT Size | 3 | 30 | 34 | 48 |

| | |
|---|---|
| Gel Time (s) | 110-125 |
| Degradation Time | 10-12 |
| (days) | |

(B)

| Components | LOT# & Specifications |
|---|---|
| 8ARM-20k-AA | 0.029-0.031 g |
| 8ARM-20k-NH2 | 0.009-0.011 g |
| 4ARM-20k-SGA | 0.079-0.081 g |
| Phosphate Buffer Powder | 0.03-0.06 g |
| Nolvasan (2% | 2.50 mL, 1% denatonium |
| chlorhexidine) | benzoate |
| LOT Size | 64 |
| Gel Time (s) | 150 |
| Degradation Time (days) | 11 |

The kit preparation time was recorded. Loading one buffer syringe took an average of 1.5 minutes, while one solids syringe took an average of 4 minutes. Vacuum sealing one kit took approximately 1.5 minutes. Thus, the time estimate for the preparation of one kit was 7 minutes, or approximately 8 kits per hour. The kit preparation time may be improved by premixing all the solids in the correct ratios such that only one mass of solids needs to be measured, and by optimizing the vacuum sealing procedure by reducing the vacuum cycle time.

All the formulations tested were easily dispensed through a 23 to 34 gauge needle. Higher gauges exhibit a lower flow rate as expected. The mixing action between the two syringes was turbulent and the introduction of a significant amount of air bubbles was apparent. The use of a syringe filter was found to remove bubbles without any change in the polymer properties.

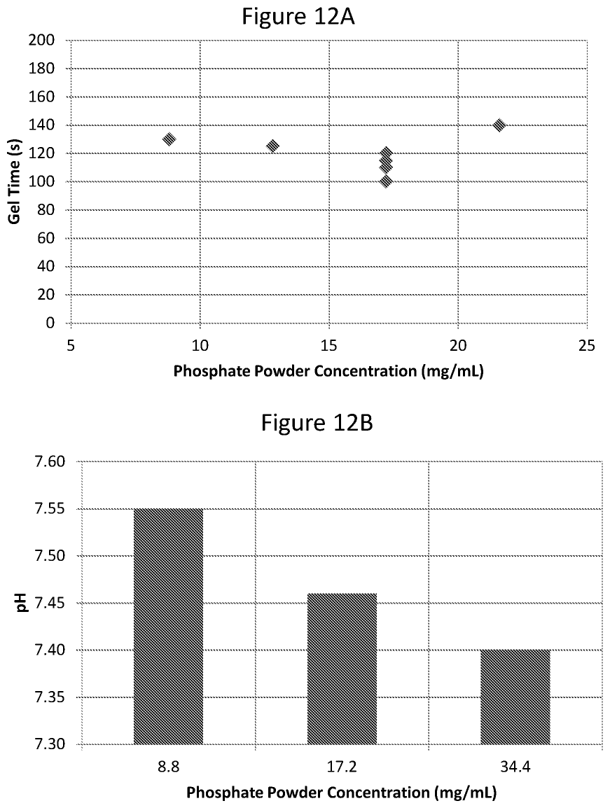

For the single syringe system, the effect of phosphate powder use was investigated. FIG. 12 shows the effect of varying amounts or concentrations of the solid phosphate on polymer gel times and solution pH. The system was found to be relatively insensitive to the amount of phosphate, tolerating up to 2-fold differences without significant variation.

### Kit Sterilization & Testing

The sealed kits were packed into large sized FedEx boxes. Each box was sterilized via electron-beam radiation at NUTEK Corporation according to a standard procedure that was developed. Included in this report is a copy of the standard sterilization procedure document.

For each lot of sterilized kits, a gel time and degradation time test was performed on a randomly selected kit to verify the viability of the materials. A previous study included a runner or control box of kits that was not sterilized, and concluded that environmental conditions during transit of the kits did not play a significant role in gel time changes.

Sterilized kits were sent to NAMSA for sterility verification according to USP<71>. The kits were verified as sterile.

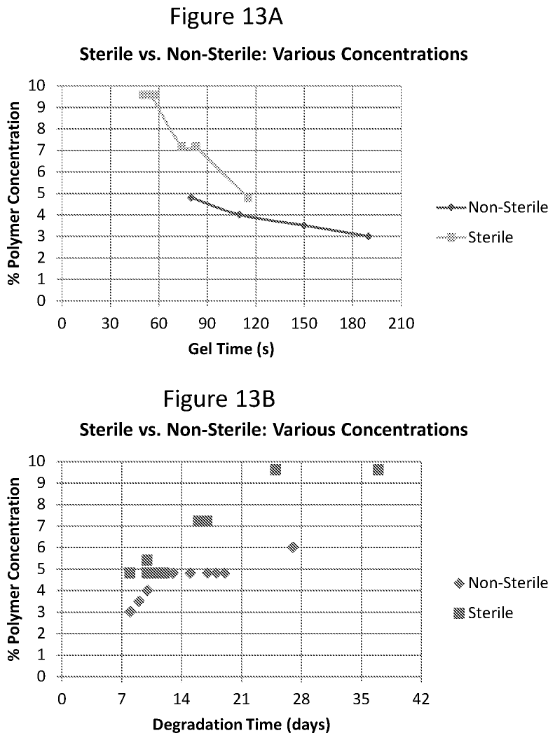

No physical changes in the monomer and phosphate buffer solutions were observed post-sterilization. Prior experiments have shown that the polymer gel times consistently increase by approximately 30 seconds after sterilization. For example, a polymer with a 90 second gel time will exhibit a 120 second gel time after sterilization. The pH of the sterile buffer was unchanged, so it was suspected that some monomer degradation during sterilization occurred. This was confirmed by preparing unsterilized polymers at various concentrations and comparing the gel times, degradation times and mechanical properties with sterilized polymers (FIG. 13). The current data shows that the monomers experience roughly 15 to 20% degradation upon sterilization. Thus, a 5% polymer after sterilization will behave similarly to a 4% polymer. Additional experiments are planned to establish a detailed quality control calibration curve.

### Storage Stability

The sterilized kits were stored at 5° C. Some kits were stored at 20° C. or 37° C. to explore the effect of temperature on storage stability. The stability of the kits was primarily quantified by recording changes in gel time, which is directly proportional to the extent of monomer degradation. The 37° C. temperature was maintained by submerging the kits fully into the water bath and thus represents the worst case scenario regarding humidity.

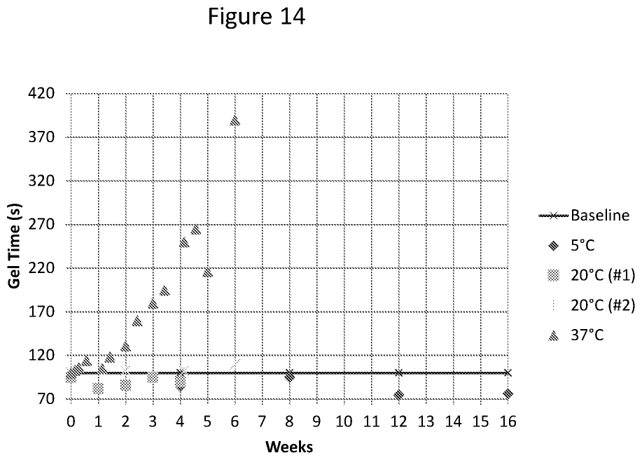

The storage stability of the kits was explored by placing some kits at 5° C., 20° C. or 37° C. and measuring the change in gel times at defined intervals. The kits were prepared and sealed according to the procedures detailed in a previous section. The results are shown in FIG. 14. Over 16 weeks, no significant change in gel times were observed for kits stored at 5° C. and 20° C. At 37° C., the gel time begins to increase after roughly 1 week at a constant rate. The foil pouch proved to be an effective moisture barrier. The indicating silica gel packet exhibited only mild signs of moisture absorption as evidenced by the color. Longer term data is still in the process of being collected.

### Example of Syringe Kit Preparation

One syringe kit was developed where the components are stored in two syringes, a male and a female syringe. The female syringe contains a mixture of white powders. The male syringe contains a buffer solution. The two syringes are connected and the contents mixed to produce a liquid polymer. The liquid polymer is then sprayed or applied over the suture wound where it covers the entire suture line. During the process, the polymer enters the voids left by sutures and protects the wound from infections. At the wound site, the liquid polymer turns into a solid gel and stays at the site for over two weeks. During this time, the wound is healed and infection free.

The components necessary to prepare the kit are disclosed in Table 17 and Table 18. To prepare the powder components of the kit to fill into the female syringe, the plunger of the 5 mL female Luer-lock syringe was removed, and the syringe was capped with the appropriate cap. 8ARM-20k-AA (0.028 g, the acceptable weight range is 0.0270 g to 0.0300 g), 8ARM-20k-NH2 (0.012 g, the acceptable weight range is 0.0100 g to 0.0130 g), 4ARM-20k-SGA (0.080 g, the acceptable weight range is 0.0790 g to 0.0820 g), and 0.043 g of freeze-dried phosphate buffer powder (0.043 g, the acceptable weight range is 0.035 g to 0.052 g) were each carefully weighed out and poured into the syringe. The syringe was then flushed nitrogen/argon gas for about 10 seconds at a rate of 5 to 10 L/min and the plunger was replaced to seal the contents. The syringe was then flipped so that the cap was facing towards the ceiling. The syringe cap was then loosened and the air space in the syringe was minimized by expelling as much air as possible from the syringe. Typical compressed powder volume is 0.2 mL. Then, the syringe cap was tighten until the cap was finger tight.

The liquid component was prepared on a 500 mL batch size, wherein 50 mL of commercial 2% chlorhexidine solution, 450 mL of distilled water, and 1.5 g of HPMC were poured in to sterile container. The sterile container was then capped and shook vigorously for 10 seconds. The solution was allowed to stand under ambient conditions for 16 hours, thereby allowing for the foam to dissipate and any remaining HPMC to dissolve.

The liquid/buffer syringe was prepared by removing the plunger of the male Luer-lock syringe followed by capping the syringe with the appropriate cap. 2.50 mL of the buffer/liquid solution was transferred by pipette into the syringe. The syringe was then flushed with nitrogen/argon gas for about 5 seconds at a rate of 5 to 10 L/min. The plunger of the syringe was then replaced to seal the contents. Then the syringe was flipped so that the cap was facing towards the ceiling and the syringe cap was loosen and air space was minimized by expelling as much air as possible from the syringe. Then the syringe cap was tightened until the cap was finger tight.

TABLE 17
Components used to fabricate the solid components
for the female syringe

| Components | Technical Name |
|---|---|
| 8ARM-20k-AA | 8ARM PEG Acetate amine, HCl |
| | salt, MW 20k |
| 8ARM-20k- | 8ARM PEG amine |
| NH2 | (hexaglycerol), HCl salt, MW 20k |
| 4ARM-20k- | 4-arm PEG succinimidyl |
| SGA | glutaramide (pentaerythritol), |
| | MW 20k |
| | Commercial 2% chlorhexidine |
| | solution |
| | Freeze-dried phosphate buffer |
| | powder |

TABLE 18
Materials used to fabricate kit including vendor, part
number and lot number.

| | | | Vendor |
|---|---|---|---|
| Description | Vendor | Part # | Catalog # |
| 10 mL Luer-Lok | BD | CM-0003 | 309604 |
| Syringe | | | |
| Non-Vented Luer | QOSINA | CM-0004 | 65119 |
| Dispenser Tip Cap, | | | |
| White | | | |
| 5 mL Female Luer- | QOSINA | CM-0005 | C3610 |
| Lock Syringe, Purple | | | |
| PP | | | |
| Male Luer Lock Cap, | QOSINA | CM-0006 | 11166 |
| Non-Vented, PP | | | |

### Example of Syringe Kit Preparation

Another syringe kit was developed where the solid components, a mixture of white powders, are stored in one female syringe. A standard male syringe is used to take up the drug solution, such as one containing Kenalog. The two syringes are connected and the contents mixed to produce a liquid polymer. The liquid polymer is then delivered to the target site.

The components necessary to prepare the kit are disclosed in Table 17 and Table 18. To prepare the powder components of the kit to fill into the female syringe, the plunger of the 5 mL female Luer-lock syringe was removed, and the syringe was capped with the appropriate cap. 8ARM-20k-AA (0.0125 g, the acceptable weight range is 0.012 g to 0.013 g), 8ARM-20k-NH2 (0.075 g, the acceptable weight range is 0.007 g to 0.008 g), 4ARM-20k-SGA (0.040 g, the acceptable weight range is 0.040 g to 0.042 g), and 0.018 g of freeze-dried phosphate buffer powder (0.043 g, the acceptable weight range is 0.017 g to 0.022 g) were each carefully weighed out and poured into the syringe. The syringe was then flushed nitrogen/argon gas for about 10 seconds at a rate of 5 to 10 L/min and the plunger was replaced to seal the contents. The syringe was then flipped so that the cap was facing towards the ceiling. The syringe cap was then loosened and the air space in the syringe was minimized by expelling as much air as possible from the syringe. Then, the syringe cap was tightened until the cap was finger tight.

### Example 14: General Procedure for the Preparation of a Polyglycol-Based, Biocompatible Hydrogel Polymer Matrix

A polyglycol-based, biocompatible pre-formulation is prepared by mixing 0.028 g of 8ARM-AA-20K, 0.012 g of 8ARM-NH2-20K, and 0.080 g of 4ARM-SGA-20K. 2.50 mL of culture medium is added to the formulation. The formulation is mixed for about 10 seconds and a 1 mL solution of the mixture is pipetted out using a mechanical high precision pipette. The polyglycol-based, biocompatible pre-formulation components polymerize to form a polyglycol-based, biocompatible hydrogel polymer matrix. The polymerization time of 1 mL liquid is collected and then verified with the lack of flow for the remaining liquids.

### Example 15: General Procedure for the Preparation of a Polyglycol-Based, Biocompatible Hydrogel Polymer Matrix and Stem Cells

A polyglycol-based, biocompatible pre-formulation is prepared by mixing 0.0125 g of 8ARM-AA-20K, 0.0075 g of 8ARM-NH2-20K, and 0.040 g of 4ARM-SGA-20K. 1.0 mL of culture medium is added to the formulation. The formulation is mixed for about 10 seconds and a 1 mL solution of the mixture is pipetted out using a mechanical high precision pipette. The polyglycol-based, biocompatible pre-formulation components polymerize to form a polyglycol-based, biocompatible hydrogel polymer matrix. The polymerization time of 1 mL liquid is collected and then verified with the lack of flow for the remaining liquids. Various sized slices of the polymerized polyglycol-based, biocompatible hydrogel polymer matrix are placed in different wells of a 24 well plate. 0.5 mL of adult mesenchymal stem cells are seeded onto the polymer matrices at various densities. The stem cells diffuse and become incorporated into the polyglycol-based, biocompatible hydrogel polymer matrix. Incorporation of the stem cells into the polyglycol-based, biocompatible hydrogel polymer matrix is demonstrated by removing a slice of the polymer matrix 10 days after stem cell addition and using the slice to expand the cells in culture. The incorporated stem cells remain viable, as demonstrated by their ability to proliferate in culture.

### Example 16: General Procedure for the Preparation of a Polyglycol-Based, Biocompatible Hydrogel Polymer Matrix and Stem Cells

A polyglycol-based, biocompatible pre-formulation is prepared by mixing 0.0125 g of 8ARM-AA-20K, 0.0075 g of 8ARM-NH2-20K, and 0.040 g of 4ARM-SGA-20K. 1.0 mL of culture medium containing adult mesenchymal stem cells is added to the formulation. The formulation is mixed for about 10 seconds and a 1 mL solution of the mixture is pipetted out using a mechanical high precision pipette. The polyglycol-based, biocompatible pre-formulation components polymerize to form a polyglycol-based, biocompatible hydrogel polymer matrix. The polymerization time of 1 mL liquid is collected and then verified with the lack of flow for the remaining liquids.

At any point during the combination of the polyglycol-based, biocompatible pre-formulation compounds, additional components may be added to the formulation. The formulation may be solid, liquid, polymerized, gelled, or any combination thereof when the additional component is added. The additional component may combine with or diffuse through the formulation and become retained with the formulation for a determined period of time. In one example, the polyglycol-based, biocompatible hydrogel polymer matrix is formed, followed by the addition of growth factors. The growth factors are incorporated into the polyglycol-based, biocompatible hydrogel polymer matrix. Additional components include, but are not limited to, biomolecules, antibiotics, anti-cancers, anesthetics, anti-virals, or immunosuppressive agents.

### Example 17: Viability of Cells in a Polyglycol-Based, Biocompatible Hydrogel Polymer Matrix

A single cell suspension of mesenchymal stem cells in D15 (DMEM, high glucose, 15% fetal bovine serum) is prepared and the cells counted. 1 mL of cells at a 2×10⁴/mL density are added to a 50 mL tube. The cells are maintained at room temperature and prepared just before addition to a pre-formulation. A female syringe containing a polyglycol-based, biocompatible pre-formulation is prepared by mixing 0.0125 g of 8ARM-AA-20K, 0.0075 g of 8ARM-NH2-20K, and 0.040 g of 4ARM-SGA-20K in the female syringe. An 18G need is attached to a male syringe and the male syringe is filled with 1 mL PBS. The next step is carried out within 90-120 seconds. The needle is removed from the male syringe and the male syringe is attached to the female syringe containing the pre-formulation. The PBS is pushed from the male syringe into the female syringe and the mixing process is started by repeatedly pushing the PBS from one syringe to the other, with 20 strokes being sufficient for mixing. After the final stroke, the entire contents are pushed into the male syringe. An 18G needle is attached to the male syringe and the liquid pre-formulation is ejected into the 50 mL tube containing the 1 mL of mesenchymal stem cells. The cells are carefully mixed while the liquid pre-formulation is being ejected into the tube. Care is made to ensure that the cells are not mixed by aspiration with the needle as this may induce cell stress.

Aliquots of the pre-formulation containing mesenchymal stem cells are placed in chambers of a 4-chamber tissue culture glass slide at 50, 100, 200 and 400 μL. The pre-formulation is allowed to gel for 2 minutes. 200 μL of D15 is added to each chamber. Three of these slides are prepared for three time points: 0, 2, and 24 hours. The cells are stained with membrane-permeant 3′,6′-Di(O-acetyl)-2′,7′-bis[N,N-bis(carboxymethyl) aminomethyl] fluorescein, tetraacetoxymethyl ester and membrane-impermeant ethidium homodimer-1, 1 μ/ml propidium iodide. The cells are imaged using brightfield and fluorescence microscopy. Live cells fluoresce green and dead cells fluoresce red. At the 2 hour time point, only one dead cell was observed in multiple field views. One live cell had a punctate cytoplasm. The remaining cells were viable and had typical spheroid morphology in the hydrogel polymer matrix. At the 24 hour time point, more than 95% of the cells were viable.

### Example 18: General Procedure to Determine the Properties of Cells in a Polyglycol-based, Biocompatible Hydrogel Polymer Matrix

The proliferation rate, viability and structural characteristics of mesenchymal stem cells are evaluated after incorporation with a biocompatible hydrogel polymer matrix.

To measure the rate of proliferation of mesenchymal stem cells, a cell proliferation assay is performed. A biocompatible pre-formulation comprising polyglycol-based compounds and a suitable buffer, as described in Example 14, is prepared. The 100 μl of the pre-formulation is coated on a 24 well plate to give a coating of <5 mm thick. The stem cells are seeded onto the coated plate at various cell densities (1×10³, 5×10³, 10×10³ and 20×10³ cells). Cells are incubated in a growth medium at 37° C., 5% CO₂. For each sample, a CellTiter 96® AQueous Non-Radioactive (MTS) assay is performed at days 2, 7, and 10 after seeding to confirm that the cells are proliferating. The growth medium is removed from each well and replaced with 500 μl of fresh medium and incubate at 37° C. for at least 1 hour in 5% CO₂. 100 μl of MTS reagent is added to each well and incubated at 37° C. for 3 hours, in 5% CO₂. The absorbance at 490 nm is measured using a microplate reader and recorded. Wells with the formulation but without any cells are used as blanks. Similarly, only media in the wells without any cells serve as blanks. Each sample reading is obtained by subtracting the blank. The graph of absorbance versus time is plotted. Absorbance is directly proportional to the cell numbers, wherein a significant increase in absorbance indicates cell viability and proliferation. Fold change in proliferation is calculated.

To demonstrate the viability of adult mesenchymal stem cells, a staining assay is performed at days 2, 7, and 10 on cells which are seeded on a coated 24 well plate as described previously in this example. The medium is removed and the cells are washed twice with phosphate saline buffer. A 0.5 ml staining solution comprising a mixture of celcein-am (10 μm/ml) and propidium iodide (100 μm/ml) is added to each well and the plate is incubated for 5-10 minutes at 37° C. Cells are washed with phosphate saline buffer and immediately imaged. Live cells fluoresce green and dead cells fluoresce red.

To demonstrate that the adult mesenchymal stem cells maintain their structure, a staining assay is performed on the cells which are seeded a coated 24 well plate as described previously in this example. The medium is removed and the cells are washed twice with phosphate buffer. The cells are fixed with 4% paraformaldehyde for 10 minutes at room temperature followed by two washes with phosphate buffer. To the washed cells, cytoplasmic WGA stain (wheat germ agglutinin; 488 green fluorescence) is added and the cells and the cells are incubated for 10 minutes at room temperature. The stain is removed and the cells are washed two times with phosphate buffer. A nuclear TO-PRO-3 iodide stain (red fluorescence) is added to the cells and the cells are incubated for 10 minutes at room temperature. The stain is removed and the cells are washed two times with HBSS buffer. The anti-fade reagent Pro-long gold is added to the cells and the cells are covered with a coverslip. 3D confocal microscopy is performed to visualize the structure and adherence of the cells. In general, the stem cells maintain their physiochemical properties.

### Example 19: Cell Elution from a Polyglycol-Based, Biocompatible Hydrogel Polymer Matrix

A polyglycol-based, biocompatible hydrogel polymer matrix of Example 15 is prepared. Additional polyglycol-based, biocompatible hydrogel polymer matrices are prepared utilizing pre-formulation compounds of Table 13 and cell. The polymer matrices are weighed and placed in different Falcon tubes. Two ml of buffer/gm of the polymer matrix are added in the falcon tubes. The falcon tubes are placed in a water bath maintained at 37° C. After 24 hours, buffer is carefully removed and replaced with fresh buffer to maintain a constant volume. The extraction process is repeated until each polymer matrix is dissolved completely. The polymer matrix is dissolved in two weeks.

The elution behavior of the cells with different biocompatible pre-formulation components is tested. Cell elution profiles vary with different biocompatible pre-formulation components. Cells may diffuse while the polymer matrix is maintained, released upon degradation of the polymer matrix or any combination thereof. The composition of the biocompatible pre-formulation components may be selected to control the release of cells at a pre-determined time.

In some instances, the cell-containing polymer matrices described herein further comprise additional components such as buffers, growth factors, antibiotics, or anti-cancer agents. The composition of the biocompatible pre-formulation components and additional components may be varied to control the release of cells and/or the additional components.

In some instances, the cells of any of the cell-containing polymer matrices described in this example may be released from the polymer matrix in a manner dependent on the pore-size of the polymer matrix. In some instances, the cells remain viable after release from the polymer matrix.

### Example 20: A Polyglycol-Based, Biocompatible Pre-Formulation for Disease Treatment

A polyglycol-based, biocompatible pre-formulation comprising, 0.0125 g 8ARM-AA-20K, 0.0075 g 8ARM-NH2-20k, 0.040 g 4ARM-SGA-20K, mesenchymal stem cells, and a suitable culture medium are combined in the presence of 1.0 mL water. The liquid formulation is delivered via injection directly to a site of tissue damage in the liver. The polyglycol-based, biocompatible pre-formulation mixture polymerizes in vivo at the site of delivery to form a polyglycol-based, biocompatible hydrogel polymer matrix at the target site in 4 minutes. The polyglycol-based, biocompatible hydrogel polymer matrix culture medium component is configured to influence the physical, chemical and biological environment surrounding the stem cells during and after administration to a target site.

The polyglycol-based, biocompatible hydrogel polymer matrix is retained at the target site, where the stem cells are released over a period of two weeks. The released stem cells require interaction and integration with the target tissue through incorporation of appropriate physical and cellular signals. Therefore, the polyglycol-based, biocompatible hydrogel polymer matrix culture medium includes modifying factors, such as biologically active proteins critical for successful tissue generation. The mesenchymal stem cells begin to differentiate at the target site between 7 and 14 days, resulting in improved liver function.

### Example 20: A Polyglycol-Based, Biocompatible Hydrogel Polymer Matrix for Disease Treatment

A polyglycol-based, biocompatible hydrogel polymer matrix is prepared by adding 1 mL of water to a pre-formulation comprising, 0.0125 g 8ARM-AA-20K, 0.0075 g 8ARM-NH2-20k, 0.040 g 4ARM-SGA-20K, mesenchymal stem cells, and a suitable culture medium. After gelling is complete, the hydrogel polymer matrix is delivered directly to a site of tissue damage in the liver. The polyglycol-based, biocompatible hydrogel polymer matrix culture medium component is configured to influence the physical, chemical and biological environment surrounding the stem cells during and after administration to the target site in the liver.

The polyglycol-based, biocompatible hydrogel polymer matrix is retained at the target site, where the stem cells are released over a period of two weeks. The released stem cells require interaction and integration with the target tissue through incorporation of appropriate physical and cellular signals. Therefore, the polyglycol-based, biocompatible hydrogel polymer matrix culture medium includes modifying factors, such as biologically active proteins critical for successful tissue generation. The mesenchymal stem cells begin to differentiate at the target site between 7 and 14 days, resulting in improved liver function.

### Example 21: A Polyglycol-Based, Biocompatible Polymer Matrix for Delivery of Growth Factors

A polyglycol-based, biocompatible pre-formulation comprising, 0.028 g 8ARM-AA-20K, 0.012 g 8ARM-NH2-20k, 0.08 g 4ARM-SGA-20K, growth factors, and a buffer are combined in the presence of 2.5 mL water. The liquid formulation is delivered via injection directly to a site of tissue damage. The polyglycol-based, biocompatible pre-formulation mixture polymerizes in vivo at the site of delivery to form a polyglycol-based, biocompatible hydrogel polymer matrix at a target site. The polyglycol-based, biocompatible hydrogel polymer matrix is configured to release the growth factors at the target site. The growth factors are configured to recruit cells from the body to the polymer matrix site, wherein the recruited cells may form tissue upon and throughout the polymer matrix.

An alternative to growth factor incorporation in a polyglycol-based, biocompatible hydrogel polymer matrix is to integrate DNA plasmids encoding a gene and mammalian promoter into the polymer matrix. Delivery of the polyglycol-based, biocompatible hydrogel polymer matrix with the DNA programs local cells to produce their own growth factors.

### Example 22: Pore Size Determination

The pore diameters are estimated from the molecular weight per arm of the combined components. The pore diameter is calculated based on the number of PEG units per arm and a carbon-carbon-carbon bond length of 0.252 nm with a 110° bond angle. This assumes a fully extended chain that accounts for bonding angles and complete reactivity of all functional end groups to form the pore network. The pore diameter is further modified by a correlation relating the pore size to the inverse of the biocompatible hydrogel swelling ratio:

L≈(Vₚ/Vₛ)^{-1/3} (Equation 1)

where Vₚ is the volume of polymer, Vₛ is the volume of the swollen gel, L is the calculated pore diameter, and is the swollen pore diameter. Based on equilibrium swelling experiments, the ratio of Vₚ to Vₛ is estimated to be around 0.5.

For the case of multi-component mixtures with a reactive ester, the weighted average of each component with the ester is used. For example, the pore sizes obtained from 4ARM-20k-AA with 4ARM-20k-SGA are averaged with the pore sizes obtained from 8ARM-20k-NH2 with 4ARM-20k-SGA for polymers comprised of 4ARM-20k-AA and 8ARM-20k-NH2 with 4ARM-20k-SGA.

### Example 23: Treatment of a Wound with a Laser

A wound is treated with a hydrogel bandage as provided for herein, such as a hydrogel form from the polymerization of 8-ARM-AA-20K, 8-ARM-NH2-20K, and 4-ARM-SGA-20K. The composition can also have a viscosity agent, such as HPMC, and a phosphate buffer. The composition can also have sodium hyaluronate. After being bandaged with the hydrogel, the wound is treated with a laser through the bandage. Surprisingly, the laser is effective to promote healing.

The present embodiments and examples demonstrate the surprising and unexpected results that demonstrate that a laser can be used through a hydrogel bandage.

1. A method of treating a wound on a subject, the method comprising contacting a wound that is covered by a hydrogel bandage with a laser pulse to treat the wound, wherein the hydrogel bandage is not removed while the laser pulse is applied to the wound.

2. The method of claim 1, wherein the hydrogel bandage is a fully synthetic, polyglycol-based biocompatible hydrogel polymer matrix comprising a fully synthetic, polyglycol-based biocompatible hydrogel polymer comprising at least one first monomeric unit bound through at least one amide, thioester, or thioether linkage to at least one second monomeric unit, wherein the polymer forms the matrix covers the wound.

3. The method of claim 2, wherein, the polyglycol-based biocompatible hydrogel polymer matrix of claim 1, wherein the at least one first monomeric unit is PEG-based and fully synthetic, and wherein the at least one second monomeric unit is PEG-based and fully synthetic.

4. The method of claim 2, wherein the first monomeric unit is derived from a MULTIARM-(5-50k)-SH, a MULTIARM-(5-50k)-NH2 or a MULTIARM-(5-50k)-AA monomer and the second monomeric unit is derived from a MULTIARM-(5-50k)-SG, a MULTIARM-(5-50k)-SGA, or a MULTIARM-(5-50k)-SS monomer.

5. The method of claim 2, wherein the first monomeric unit is derived from a 4ARM-5k-SH, 4ARM-2k-NH2, 4ARM-5k-NH2, 8ARM-20k-NH2, 4ARM-20k-AA, or 8ARM-20k-AA monomer, and the second monomeric unit is derived from a 4ARM-10k-SG, 8ARM-15k-SG, 4ARM-20k-SGA, or 4ARM-20k-SS monomer.

6. The method of claim 1, wherein the hydrogel bandage comprises a hydrogel formed from a mixture of 8-ARM-AA-20K, 8-ARM-NH2-20K, and 4-ARM-SGA-20K.

7. (canceled)

8. (canceled)

9. The method of claim 1, wherein the hydrogel is prepared from a fully synthetic polyglycol-based biocompatible pre-formulation, comprising:
(a) at least one fully synthetic polyglycol-based first compound comprising more than one nucleophilic group; and
(b) at least one fully synthetic polyglycol-based second compound comprising more than one electrophilic group;
wherein the polyglycol-based biocompatible pre-formulation at least in part polymerizes and/or gels to form a polyglycol-based biocompatible hydrogel polymer matrix at the site of the wound site.

10. The method of claim 9, wherein, the first compound of the pre-formulation is a MULTIARM-(5-50k)-SH, a MULTIARM-(5-50k)-NH2, a MULTIARM-(5-50k)-AA, or a combination thereof, and the second compound of the pre-formulation is a MULTIARM-(5-50k)-SG, a MULTIARM-(5-50k)-SGA, a MULTIARM-(5-50k)-SS, or a combination thereof.

11. The method of claim 10, wherein the first compound is 4ARM-5k-SH, 4ARM-2k-NH2, 4ARM-5k-NH2, 8ARM-20k-NH2, 4ARM-20k-AA, 8ARM-20k-AA, and a combination thereof, and the second compound is 4ARM-10k-SG, 8ARM-15k-SG, 4ARM-20k-SGA, 4ARM-20k-SS, or a combination thereof.

12. The method of claim 10, wherein the first compound is 8ARM-20k-NH2 and/or 8ARM-20k-AA, and the second compound is 4ARM-20k-SGA.

13. The method of claim 10, wherein the polyglycol-based biocompatible pre-formulation gels to form a polyglycol-based biocompatible hydrogel polymer matrix in between about 20 seconds and 10 minutes at the site of the wound.

14. (canceled)

15. (canceled)

16. The method of claim 6, wherein the ratio of 8ARM-20k-AA to 8ARM-20k-NH2 is about 1:1, 70:30, or 75:25.

17. The method of claim 1, wherein the laser pulse is at a wavelength of about 630 to about 685 nm or about 700 to about 1000 nm.

18. (canceled)

19. (canceled)

20. The method of claim 1, wherein the laser pulse is pulsed for about 1 to about 999 milliseconds.

21. (canceled)

22. (canceled)

23. The method of claim 1, wherein the laser pulse is administered to the subject through the hydrogel bandage.

24. The method of claim 1, wherein the laser pulse is administered through the bandage once a day for 5, 6, 7, 8, 9, 10, 11, 12, 13, or 14 days.

25. The method of claim 1, wherein the laser pulse is administered at a dose of about 1 J/cm² to about 5 J/cm².

26. (canceled)

27. (canceled)

28. (canceled)

29. The method of claim 1, wherein the wound is a burn, traumatic injury, a cut, laceration, abrasion, puncture, or avulsion.

30. The method of claim 1, wherein the hydrogel bandage comprises silver.

31. The method of claim 1, wherein the hydrogel bandage is free of any active ingredients.

32. (canceled)

33. (canceled)

34. (canceled)

35. (canceled)
